(19) United States
(12) Patent Application Publication
(10) Pub. No.: US 2024/0376482 A1
(43) Pub. Date: Nov. 14, 2024

(54) METHOD OF MAKING POLYHYDROXYALKANOATE COPOLYMERS FROM DIVERSE SUBSTRATES

(71) Applicant: The Research Foundation for The State University of New York, Albany, NY (US)

(72) Inventors: Christopher Thomas, Syracuse, NY (US); Ryan Scheel, Rochester, NY (US); Bandaru Ramarao, Fayetteville, NY (US); Christopher Nomura, Syracuse, NY (US); Deepak Kumar, Jamesville, NY (US)

(21) Appl. No.: 18/291,218

(22) PCT Filed: Jul. 22, 2022
(86) PCT No.: PCT/US2022/038048

§ 371 Date: Jan. 22, 2024

Related U.S. Application Data
(60) Provisional application No. 63/225,074, filed on Jul. 23, 2021.

(51) Int. Cl. C12N 15/52; C12N 1/20; C12N 9/04; C12N 9/10; C12N 9/14; C12P 7/625

(52) U.S. Cl. CPC C12N 15/52; C12N 1/205; C12N 9/0006; C12N 9/1029; C12N 9/14; C12P 7/625; C12Y 101/01036; C12Y 203/01009; C12Y 402/01017; C12N 2510/02

(57) ABSTRACT

The present disclosure provides a microorganism and expression cassette useful for biologically producing PHA ho-mopolymers and/or PHA copolymers, including PHB-co-MCL copolymers of controllable or predetermined composition. In embodiments, the present disclosure provides a nucleic acid construct suitable for use in a microorganism and/or expression cassette including a nucleic acid construct including: one or more genes comprising a phaJ4 gene, a phaA gene, a phaB gene, a phaC1 gene, or combinations thereof; a cDNA that encodes one or more proteins comprising an enoyl-CoA hydratase 2, a β-ketothiolase, an acetoacetyl-CoA reductase, a type II poly hydroxyalkanoate synthase, or combinations thereof; or one or more nucleic acid sequences that encode one or more proteins including an enoyl-CoA hydratase 2, a β-ketothiolase, an acetoacetyl-CoA reductase, a type II poly hydroxyalkanoate synthase, or combinations thereof.

### CROSS REFERENCE TO RELATED APPLICATIONS

The present application is a National Stage Application under 35 U.S.C. § 371, of Patent Cooperation Treaty Application No. PCT/US2022/038048, filed Jul. 22, 2022, published as WO 2023/004135, which claims benefit of priority from U.S. Provisional Patent Application No. 63/225,074, filed Jul. 23, 2021, the entirety of which are expressly incorporated herein by reference.

### REFERENCE TO A SEQUENCE LISTING

This application contains a Sequence Listing in computer readable form, which is incorporated herein by reference.

### SEQUENCE LISTING

This application contains a Sequence Listing that has been filed electronically in the form of a XML file, created Jul. 22, 2022, and named “SUNY-ESF-2158-550PCT.xml” and is 33,657 bytes in size, the contents of which are incorporated herein by reference in their entirety.

### FIELD OF THE INVENTION

The invention relates to the field of genetic engineering and provides novel DNA molecules and engineered microorganisms. For example, one or more strains of Escherichia coli (E. coli) including one or more DNA molecules for use as recombinant vectors suitable for biologically producing polyhydroxyalkanoate (PHA) homopolymers and/or PHA copolymers, including PHB-co-MCL copolymers of controllable or predetermined composition. In embodiments, the present disclosure relates to biodegradable poly (3-hydroxyalkanoate) copolymers and methods for synthesizing biodegradable poly (3-hydroxyalkanoate) copolymers.

### BACKGROUND OF THE INVENTION

Poly (3-hydroxyalkanoates) (PHAs) are biodegradable plastics and carbon storage materials synthesized by a myriad of microorganisms. PHAs are generally divided into groups based on repeating unit size. For example, PHAs with repeating units of three to five carbons in length are short-chain-length (SCL) PHAs, and PHAs with repeating units of six to fourteen carbons in length are medium-chain-length (MCL) PHAs. Differences in repeating unit composition influence the physical properties of PHAs. SCL PHA homopolymers such as poly-3-hydroxybutyrate (PHB) have been previously produced, but methods to control the repeating unit composition of the MCL PHAs have been limited. Previous attempts to control MCL PHA synthesis in native and recombinant PHA-producing organisms have resulted in narrow ranges of repeating unit control. Typically, control is limited to only a couple of repeating units within one organism or control is lost once the number of carbons in the repeating unit exceeds seven.

Thus, the composition of medium-chain-length (MCL) poly (R)-3-hydroxyalkanoate) (PHA) biopolymers is normally an uncontrollable random mixture of repeating units with differing side chain lengths. Attempts to generate MCL PHA homopolymers and gain control of the repeating unit composition have been reported for native or natural PHA-producing organisms but have limited ranges for the different sizes of repeating units that can be synthesized. The inventors have found this problematically limits the range of mechanical properties and applicability of current PHA plastics.

Further, sustainable production of PHA bioplastics is problematic. Although there are PHA plastics currently on the market, current technologies limit the range of mechanical properties and therefore applicability of current PHA plastics. These limitations greatly hinder the market penetration of PHA plastics. The inventors have found technology is needed that allows for the production of PHA plastics better able to compete with petroleum-based plastics in terms of both price and performance.

PHAs are biodegradable (not industrially compostable, as with PLA) plastics. Successful deployment of this technology would see replacement of conventional single-use consumer plastics with PHA. Replacement of these difficult-to-recycle plastics with PHA-based plastics would reduce the burden on municipal recycling and waste management services, and also divert hundreds of millions of tons of non-biodegradable plastic waste from entering the environment and aquatic ecosystems, breaking down into microplastics, collecting environmental toxins, entering the food chain and ultimately human bodies (with to-date unknown consequences). Importantly, this technology confers the ability to produce diverse PHA copolymers from waste streams, which not only makes for a more sustainable and economically competitive process, but also would increase the value of large organic waste streams while diverting them from landfills and/or low value uses.

Prior art of interest includes U.S. Pat. No. 10,017,794 entitled Engineered Strain of Escherichia coli for Production of Poly-R-3-Hydroxyalkanoate Polymers with Defined Monomer Unit Compositions and Methods Based Thereon to Nomura et al. (herein entirely incorporated by reference). However, this reference does not overcome deficiencies of producing PHA copolymers of the present disclosure, e.g., the strains therein require a butyric acid precursor to make (R)-3-poly((R)-3-hydroxybutyrate) (PHB) an inefficient precursor limitation. Moreover, the system does not provide for controllable biosynthesis of a diverse array of polyhydroxybutyrate (PHB) based polyhydroxyalkanoatc (PHA) copolymers in recombinant Escherichia coli from a diverse group of substrates.

U.S. Pat. No. 7,371,558 entitled Process for the biological production of 1,3 propanediol with high yield to Cervine et al. (herein entirely incorporated by reference) relates to using genetically modified bacteria for production of products. However, the patent relates to different strains and different vectors and are not suitable for controllable biosynthesis of a diverse array of polyhydroxybutyrate (PHB) based polyhydroxyalkanoate (PHA) copolymers in recombinant Escherichia coli.

U.S. Pat. No. 7,135,316 entitled Escherichia coli having accession No. PTA 1579 and its use to produce polyhydroxybutyrate to Mahishi, et al. (herein entirely incorporated by reference) relates to using genetically modified bacteria for production of products such as PHB. However, the patent relates to different strains and different vectors and are not suitable for controllable biosynthesis of a diverse array of polyhydroxybutyrate (PHB) based polyhydroxyalkanoate (PHA) copolymers in recombinant Escherichia coli in accordance with the present disclosure.

Accordingly, there is a need for improved vectors, strains, and methods for controllable biosynthesis of a diverse array of polyhydroxybutyrate (PHB) based polyhydroxyalkanoate (PHA) copolymers in recombinant Escherichia coli from a diverse group of substrates. Each of the following patents and patent applications is expressly incorporated herein by reference in its entirety: AU-1942999; AU-2002236464; AU-2003243831; AU-2007322528; AU-2007322528; AU-2007322529; AU-2007322529; AU-2009269147; AU-2009269147; AU-2009269147; AU-2010295637; AU-2010295637; AU-2010295638; AU-2013213729; AU-2019309401; AU-2019324739; AU-2020293986; AU-4027700; AU-5368800; AU-5499100; AU-722033; AU-751610; AU-751673; AU-770120; AU-778081; BR-0207356; BR-112012020132; BR-112012020132; BR-112021001171; BR-112021003402; BR-PI0718974; BR-PI0718979; BR-PI0718979; BR-PI0913875; BR-PI1011947; CA-2234965; CA-2314151; CA-2368470; CA-2375831; CA-2428713; CA-2462942; CA-2729302; CA-2765800; CA-2773703; CA-2773707; CA-2781699; CA-2781699; CA-2788596; CA-2788811; CA-2869891; CA-2880726; CA-2937569; CA-3107311; CA-3110841; CA-3141340; CN-100593571; CN-101058799; CN-101553519; CN-101553519; CN-101616954; CN-101616954; CN-101665801; CN-101665801; CN-102007202; CN-102066560; CN-102066560; CN-102131935; CN-102171356; CN-102216463; CN-102216463; CN-102317437; CN-102317437; CN-102482651; CN-102781901; CN-102781901; CN-102781926; CN-102781926; CN-103396978; CN-103725717; CN-104046659; CN-104046659; CN-104250627; CN-104250628; CN-104321427; CN-104450808; CN-104520433; CN-104603277; CN-104685058; CN-105531308; CN-105754966; CN-106459544; CN-106574279; CN-107384846; CN-108602944; CN-1089564; CN-109153998; CN-109265943; CN-109265943; CN-109266597; CN-109266597; CN-110691847; CN-111032727; CN-111032876; CN-111443069; CN-111615555; CN-111705028; CN-112368385; CN-112601809; CN-112739214; CN-113330068; CN-113699189; CN-113999869; CN-114206977; CN-114703067; CN-1564868; CN-1800349; CN-1800349; CN-1810968; DE-60016555; DE-60021537; DE-60036863; DE-60123862; DE-69637253; EP-0859858; EP-0859858; EP-0881293; EP-0977865; EP-1042388; EP-1042388; EP-1044278; EP-1120461; EP-1120461; EP-1138770; EP-1138770; EP-1149912; EP-1149912; EP-1154021; EP-1154021; EP-1161539; EP-1163019; EP-1172438; EP-1172438; EP-1196614; EP-1253160; EP-1253160; EP-1334181; EP-1334181; EP-1441031; EP-1457554; EP-1659142; EP-1659142; EP-1867348; EP-1867348; EP-2084209; EP-2084209; EP-2087025; EP-2087025; EP-2196484; EP-2196484; EP-2258742; EP-2284261; EP-2284261; EP-2289967; EP-2289967; EP-2305324; EP-2305324; EP-2307552; EP-2377945; EP-2377945; EP-2395075; EP-2443163; EP-2443163; EP-2445965; EP-2471910; EP-2477477; EP-2478105; EP-2510037; EP-2534125; EP-2534125; EP-2534125; EP-2534141; EP-2534141; EP-2540835; EP-2540835; EP-2551349; EP-2650372; EP-2650372; EP-2684949; EP-2684949; EP-2694663; EP-2702138; EP-2742143; EP-2754717; EP-2756082; EP-2812438; EP-2812438; EP-2818561; EP-2818562; EP-2855687; EP-2855687; EP-2886643; EP-2895613; EP-2924122; EP-2924122; EP-2927325; EP-2963119; EP-2963120; EP-2963120; EP-2971021; EP-2989113; EP-2993993; EP-3077501; EP-3077501; EP-3101129; EP-3101129; EP-3124544; EP-3124615; EP-3124615; EP-3187590; EP-3187590; EP-3189130; EP-3305897; EP-3358002; EP-3492581; EP-3601547; EP-3677613; EP-3677685; EP-3741840; EP-3741858; EP-3826468; EP-3831951; EP-3841169; EP-3967747; EP-3980546; EP-3986606; EP-3992226; ES-2243235; ES-2294871; ES-2294871; ES-2340915; ES-2356350; ES-2394953; ES-2395057; ES-2448823; ES-2639413; IL-280330; IL-281041; IN-1990MU2014; IN-2008KN05043; IN-2010DN09365; IN-2014MU01990; IN-2014MU02028; IN-2015CN06634; IN-202117012768; IN-2028MU2014; IN-313513; JP-2000516813; JP-2001008689; JP-2001046074; JP-2001275671; JP-2001275672; JP-2001275673; JP-2001275674; JP-2001517070; JP-2002199890; JP-2003011312; JP-2003015359; JP-2003503033; JP-2004018723; JP-2004254536; JP-2004315783; JP-2004333710; JP-2004534507; JP-2006204253; JP-2006204256; JP-2007125004; JP-2007125043; JP-2007222185; JP-2007277574; JP-2008194049; JP-2008245633; JP-2008248258; JP-2009112315; JP-2009509530; JP-2010510775; JP-2011519561; JP-2011522072; JP-2011526943; JP-2012152171; JP-2012501678; JP-2012511912; JP-2012516695; JP-2012529267; JP-2013507145; JP-2013510572; JP-2014147400; JP-2014518613; JP-2015006181; JP-2015006182; JP-2015029484; JP-2015051002; JP-2015211681; JP-2015221043; JP-2015512648; JP-2016105716; JP-2016155936; JP-2017029082; JP-2017077184; JP-2017205116; JP-2018046843; JP-2018074927; JP-2018521674; JP-2019119806; JP-2019500437; JP-2020005647; JP-2020010685; JP-2020031631; JP-2020058391; JP-2020508665; JP-2021087376; JP-2021530555; JP-2021534820; JP-3536050; JP-3684175; JP-3848045; JP-3848046; JP-3848047; JP-3848048; JP-3880567; JP-4024676; JP-4121541; JP-4264819; JP-4422776; JP-4532784; JP-4579502; JP-5396639; JP-5550066; JP-5624980; JP-5625905; JP-5626735; JP-5738594; JP-5770099; JP-5807878; JP-5885148; JP-5912529; JP-5964747; JP-5995109; JP-6195296; JP-6223386; JP-6478697; JP-6663221; JP-6719135; JP-6755515; JP-6772417; JP-6854909; JP-6860489; JP-6889553; JP-6990169; JP-6994057; JP-7001596; JP-WO2003033707; JP-WO2003050277; JP-WO2009131186; JP-WO2009147918; JP-WO2010050470; JP-WO2011105379; JP-WO2014133088; JP-WO2015115619; JP-WO2015146194; JP-WO2015146195; JP-WO2016021604; JP-WO2016194771; JP-WO2017033652; JP-WO2017051896; JP-WO2017056442; JP-WO2017163518; JP-WO2019044836; JP-WO2019044837; JP-WO2019142717; JP-WO2019142845; JP-WO2019240155; KR-100479943; KR-100479945; KR-100481125; KR-100507127; KR-100532721; KR-100938761; KR-101518040; KR-101578959; KR-101886012; KR-102060641; KR-102177736; KR-102311152; KR-20010095181; KR-20010095189; KR-20010095191; KR-20020083519; KR-20030013244; KR-20090050793; KR-20110104492; KR-20120103621; KR-20140027195; KR-20140070605; KR-20140117733; KR-20150000839; KR-20150000840; KR-20150004830; KR-20160025043; KR-20170113101; KR-20170132896; KR-20200046051; KR-20200046052; KR-20210031319; KR-20210038579; KR-20210048625; KR-20210049865; KR-20220085818; MX-2011000072; MX-2012009142; MX-2013011422; MX-PA00008674; MX-PA01001049; MX-PA04003275; NL-1008497; PT-1042388; PT-1163019; PT-1603987; RU-2439233; RU-2758138; TW-200918669; TW-201120213; TW-201410865; TW-201920651; TW-201920680; U.S. Pat. Nos. 10,005,880; 10,017,794; 10,072,255; 10,364,318; 10,519,473; 10,538,791; 10,717,996; 10,745,722; 10,786,064; 10,808,262; 10,829,793; 10,865,429; 10,982,240; 11,186,831; 11,225,676; 11,279,957; US-20010046692; US-20010053544; US-20010055795; US-20020086377; US-20020090687; US-20020098565; US-20020156150; US-20020173558; US-20030017576; US-20030049806; US-20030077746; US-20030082777; US-20030087413; US-20030092141; US-20030104302; US-20030124692; US-20030236320; US-20040214783; US-20040259026; US-20050009155; US-20050009156; US-20050009157; US-20050009949; US-20050107578; US-20060030014; US-20060040196; US-20060172400; US-20060252139; US-20060253937; US-20060260006; US-20070020739; US-20070027291; US-20070135929; US-20070269872; US-20080051490; US-20080095823; US-20090271889; US-20100035313; US-20100222545; US-20110040058; US-20110046339; US-20110081692; US-20110104768; US-20110166318; US-20110212497; US-20110237769; US-20120045801; US-20120060413; US-20120174253; US-20120180162; US-20120214213; US-20120315681; US-20130045516; US-20130071892; US-20130253141; US-20130302867; US-20140024769; US-20140073022; US-20140080983; US-20140114082; US-20140170714; US-20140234944; US-20140242650; US-20140295507; US-20140302572; US-20140349353; US-20140357746; US-20140357788; US-20140371417; US-20140378646; US-20140378647; US-20150148513; US-20150159184; US-20150203620; US-20150203878; US-20150225329; US-20150232886; US-20150275236; US-20150291985; US-20160040172; US-20160040197; US-20160076064; US-20160177348; US-20160194584; US-20170009008; US-20170009221; US-20170159075; US-20170183694; US-20170198136; US-20170198313; US-20170218411; US-20170306358; US-20180010156; US-20180163236; US-20180201961; US-20180305722; US-20180327544; US-20190002933; US-20190153486; US-20200056214; US-20200087634; US-20200255589; US-20200340020; US-20200347416; US-20200354696; US-20200400538; US-20210238609; US-20210254028; US-20210289794; US-20210317302; US-20210324429; US-20210332393; US-20220042025; U.S. Pat. Nos. 5,891,686; 6,083,729; 6,091,002; 6,117,658; 6,143,952; 6,228,623; 6,485,951; 6,492,134; 6,514,743; 6,548,569; 6,610,764; 6,692,945; 6,709,848; 6,803,219; 6,803,220; 6,808,910; 6,812,013; 6,828,357; 6,838,493; 6,867,247; 6,867,248; 6,872,564; 6,875,596; 6,878,758; 6,951,747; 6,972,192; 7,101,696; 7,153,622; 7,176,349; 7,179,883; 7,235,396; 7,268,205; 7,276,361; 7,341,856; 7,429,469; 7,452,960; 7,553,923; 7,786,355; 8,273,852; 8,334,129; 8,383,379; 8,394,613; 8,420,759; 8,487,159; 8,546,125; 8,546,506; 8,563,281; 8,685,701; 8,748,137; 8,765,402; 8,901,262; 8,940,849; 8,980,615; 9,084,467; 9,169,501; 9,181,559; 9243265; 9,249,252; 9,296,850; 9,353,238; 9,447,441; 9,663,791; 9,816,111; 9,920,338; WO-1997015681; WO-1997015681; WO-1998036078; WO-1998054329; WO-1998054329; WO-1999032536; WO-1999035278; WO-2000078985; WO-2001011014; WO-2001023596; WO-2002040690; WO-2002040690; WO-2003051813; WO-2003100055; WO-2004041905; WO-2004097530; WO-2007100831; WO-2008062995; WO-2008062996; WO-2008135065; WO-2009131186; WO-2009143706; WO-2009147918; WO-2010005525; WO-2010050470; WO-2010148144; WO-2010151798; WO-2011034945; WO-2011034946; WO-2011034946; WO-2011069244; WO-2011100601; WO-2011100608; WO-2011105379; WO-2012037324; WO-2012135789; WO-2012149162; WO-2012165131; WO-2012170793; WO-2013023140; WO-2013040383; WO-2013106464; WO-2013184602; WO-2014027129; WO-2014043182; WO-2014047209; WO-2014106122; WO-2014133088; WO-2014152434; WO-2014176514; WO-2014176515; WO-2015084633; WO-2015115619; WO-2015133468; WO-2015146194; WO-2015146195; WO-2016021604; WO-2016034691; WO-2016194771; WO-2017033652; WO-2017051896; WO-2017056442; WO-2017087278; WO-2017163518; WO-2017171260; WO-2018021046; WO-2018183640; WO-2019044836; WO-2019044837; WO-2019240155; WO-2020023699; WO-2020037394; WO-2020251932; WO-2020255108; WO-2021049910; WO-2021158657; WO-2021206155; and WO-2022032169.

### SUMMARY OF THE INVENTION

The present disclosure relates to controllable biosynthesis of a diverse array of polyhydroxybutyrate (PHB) based polyhydroxyalkanoate (PHA) copolymers in recombinant Escherichia coli LSBJ and recombinant Escherichia coli LSBJ Crp* as well as diverse and controllable production of PHA homopolymers and copolymers from direct fatty acid precursors, including the production of (R)-3-poly((R)-3-hydroxybutyrate) (PHB) from non-fatty acid non-related precursors.

In embodiments, the present disclosure includes a nucleic acid construct including: one or more genes including a phaJ4 gene, a phaA gene, a phaB gene, a phaC1 gene, or combinations thereof; a cDNA encoding one or more proteins including an enoyl-CoA hydratase 2, a β-ketothiolasc, an acetoacetyl-CoA reductase, a type II polyhydroxyalkanoate synthase, or combinations thereof; or one or more nucleic acid sequences encoding one or more proteins including an (R)-specific enoyl-CoA hydratase such as enoyl-CoA hydratase 2, a β-ketothiolase, an acetoacetyl-CoA reductase, a type II polyhydroxyalkanoate synthase, or combinations thereof.

In embodiments, the present disclosure includes an expression vector including: a) a promoter sequence; and b) a nucleic acid construct including: one or more genes including a phaJ4 gene, a phaA gene, a phaB gene, a phaC1 gene, or combinations thereof; a cDNA encoding one or more proteins including an enoyl-CoA hydratase 2, a β-ketothiolase, an acetoacetyl-CoA reductase, a type II polyhydroxyalkanoate synthase, or combinations thereof; or one or more nucleic acid sequences encoding one or more proteins including an enoyl-CoA hydratase 2, a β-ketothiolase, an acetoacetyl-CoA reductase, a type II polyhydroxyalkanoate synthase, or combinations thereof. In embodiments, the present disclosure includes one or more host cells including the expression vectors and/or nucleic acid constructs of the present disclosure.

In embodiments, the present disclosure includes a method of making PHA homopolymers and/or PHA copolymers, including: (a) culturing a host cell of the present disclosure in a culture medium under conditions suitable for bioconverting a substrate to PHA homopolymers and/or PHA copolymers; and (b) isolating PHA or PHA copolymer from the culture medium or from the host cell.

In embodiments, the present disclosure includes a method of forming polyhydroxyalkanoate (PHA) homopolymers and/or PHA copolymers, including: cultivating a recombinant E. coli cell in a medium conducive for a production of the polyhydroxyalkanoate (PHA) homopolymers and/or PHA copolymers, wherein the recombinant E. coli cell includes a disrupted endogenous β-oxidation pathway including a genetically disrupted endogenous fadB gene and/or a genetically disrupted fadJ gene, and a nucleic acid construct comprising a phaJ4 gene, cDNA, or nucleic acid encoding active enoyl-CoA hydratase 2, at least one phaA gene, cDNA, or nucleic acid encoding active β-ketothiolase, at least one phaB gene, cDNA, or nucleic acid encoding active acetoacetyl-CoA reductase, and at least one phaC1 gene, cDNA, or nucleic acid encoding active type II polyhydroxyalkanoate synthase, wherein cultivating occurs under conditions suitable for bioconverting a substrate to PHA homopolymers and/or PHA copolymers.

In embodiments, the present disclosure includes one or more E. coli cells including a disrupted endogenous β-oxidation pathway including a genetically disrupted endogenous fadB gene and/or a genetically disrupted fadJ gene, and a nucleic acid construct including a phaJ4 gene encoding active enoyl-CoA hydratase 2, at least one phaA gene encoding active β-ketothiolase, at least one phaB gene encoding active acetoacetyl-CoA reductase, and at least one phaC1 (STQK) gene encoding active type II polyhydroxyalkanoate synthase.

In embodiments, the present disclosure includes one or more E. coli strains including: i) a disrupted endogenous β-oxidation pathway including a genetically disrupted endogenous fadB gene and/or a genetically disrupted fadJ gene resulting in a buildup of one or more enoyl-CoA intermediates; ii) a genetically upregulated phaJ4 gene encoding active enoyl-CoA hydratase 2 with the upregulation resulting in an increased R-(R)-(3)-hydroxyacyl-CoA equivalents, wherein the upregulation is produced by introducing at least one phaJ4 gene into the E. coli strain; iii) a genetically upregulated phaA gene encoding active β-ketothiolase with the upregulation resulting in an increased availability of (R)-3-hydroxybutyryl-CoA, wherein the upregulation is produced by introducing at least one phaA gene into the E. coli strain; iv) a genetically upregulated phaB gene encoding active acetoacetyl-CoA reductase the upregulation resulting in an increased (R)-3-hydroxybutyryl-CoA, wherein the upregulation is produced by introducing at least one phaB gene into the E. coli strain; and v) a genetically upregulated phaC1 gene encoding active type II polyhydroxyalkanoate synthase, the upregulation resulting in an increased PHA, wherein the upregulation is produced by introducing at least one phaC1 gene into the E. coli strain, wherein said E. coli strain is capable of bioconverting a suitable substrate to PHA homopolymers and/or PHA copolymers. In embodiments, the E. coli strain is characterized as not having the capability to degrade PHAs.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

The invention can be more fully understood from the following detailed description, the FIGS. 1-23, and the accompanying sequence listing and descriptions.

FIG. 1 depicts an engineering metabolic pathway for the production of PHB-co-MCL PHA copolymers in E. coli LSBJ harboring pBBRSTQKJ4 in accordance with the present disclosure.

FIGS. 2A and 2B depict concentration of glucose, xylose, and arabinose sugars in the brewer's spent grains (BSG) enzymatic hydrolysate.

FIG. 3 depicts a cell titer and PHA titer (lines) and % PHA inclusion (markers) after 48 h of E. coli LSBJ STJ4 shake flask growth with varying ratios of glucose to decanoate starting substrate in accordance with the present disclosure.

FIG. 4 depicts PHB-co-PHD copolymer ratios (bars) and final pH (line) after 48 h of E. coli LSBJ STJ4 shake flask growth with varying ratios of glucose to decanoate starting substrate in accordance with the present disclosure.

FIG. 5 depicts a cell titer and PHA titer (lines) and % PHA inclusion (markers) after 48 h of E. coli LSBJ STJ4 shake flask growth with varying ratios of hydrolysate sugars to decanoate starting substrate in accordance with the present disclosure.

FIG. 6 depicts PHB-co-PHD copolymer ratios (bars) and final pH (line) after 48 h of E. coli LSBJ STJ4 shake flask growth with varying ratios of hydrolysate sugars to decanoate starting substrate in accordance with the present disclosure.

FIG. 7 depicts H¹-NMR spectra of purified polymer produced by engineered E. coli LSBJ.

FIG. 8 depicts C¹³-NMR spectra of purified polymer produced by engineered E. coli LSBJ.

FIG. 9 depicts a plasmid map of pBBRSTQKABJ4 displaying location and expression control mechanisms of phaJ4, PhaC1, PhaA, and PhaB in accordance with the present disclosure. pBBR1 oriV (SEQ ID NO: 4): Replication origin for the pBBR1 vector, requires pBBR1 Rep protein pBBR1 Rep (SEQ ID NO: 5): Codes for the replication protein for the pBBR1 vector. phaCAB downstream: 3′ non-coding region from the Cupriavidus necator (C. necator) phaCAB operon. phaB (SEQ ID NO: 6): Codes for C. necator PhaB acetoacetyl-CoA reductase. phaA (SEQ ID NO: 7): Codes for C. necator PhaA β-ketothiolase. phaC1 (STQK) (SEQ ID NO: 8): Codes for a Pseudomonas sp. 61-3 PhaC PHA synthase, which has been modified for enhanced uptake of SCL acyl-CoA substrates. R. eutropha phaCAB upstream: 5′ non-coding region from the C. necator phaCAB operon which contains the C. necator promoter. R. eutropha promoter (SEQ ID NO: 9): A constitutive promoter from C. necator that recruits RNA polymerase to begin transcription. lacZa: The lacZα fragment of β-galactosidase (a relic of blue-white screening). phaJ4 (SEQ ID NO: 10): Codes for Pseudomonas putida PhaJ4 (R)-specific enoyl-CoA hydratase. lac operator: Prevents RNA polymerase from binding to the plasmid, and is negatively controlled by isopropylthiogalactosidase (IPTG) to begin transcription of the operon. lac promoter: Recruits RNA polymerase to begin transcription. CAP binding site: Binding site for catabolite activator protein, which activates transcription in the presence of cAMP. NcoR/KanR: Codes for resistance to kanamycin antibiotic, for strain selection and culture maintenance purposes.

FIG. 10 shows cell titer and PHA titer (lines) and % PHA inclusion (markers) after 48 h of E. coli ptsG STJ4 shake flask growth with varying concentrations of sodium dodecanoate starting substrate.

FIG. 11 shows PHB-co-PHDD comonomer ratios (bars) and final pH (line) after 48 h of E. coli ptsG STJ4 shake flask growth with varying concentrations of sodium dodecanoate starting substrate.

FIG. 12 shows cell titer and PHA titer (lines) and % PHA inclusion (markers) after 48 h of E. coli ptsG STJ4 shake flask growth with varying concentrations of sodium decanoate starting substrate.

FIG. 13 shows PHB-co-PHD comonomer ratios (bars) and final pH (line) after 48 h of E. coli ptsG STJ4 shake flask growth with varying concentrations of sodium decanoate starting substrate.

FIG. 14 shows cell titer and PHA titer (lines) and % PHA inclusion (markers) after 48 h of E. coli ptsG STJ4 shake flask growth with varying concentrations of sodium octanoate starting substrate, plus Brij-35.

FIG. 15 shows PHB-co-PHO comonomer ratios (bars) and final pH (line) after 48 h of E. coli ptsG STJ4 shake flask growth with varying concentrations of sodium octanoate starting substrate, plus Brij-35.

FIG. 16 shows cell titer and PHA titer (lines) and % PHA inclusion (markers) after 48 h of E. coli ptsG STJ4 shake flask growth with varying concentrations of sodium octanoate starting substrate.

FIG. 17 shows PHB-co-PHO comonomer ratios (bars) and final pH (line) after 48 h of E. coli ptsG STJ4 shake flask growth with varying concentrations of sodium octanoate starting substrate, plus Brij-35.

FIG. 18 shows cell titer and PHA titer (lines) and % PHA inclusion (markers) after 48 h of E. coli ptsG STJ4 shake flask growth with varying concentrations of sodium hexanoate starting substrate, plus Brij-35.

FIG. 19 shows PHB-co-PHHx comonomer ratios (bars) and final pH (line) after 48 h of E. coli ptsG STJ4 shake flask growth with varying concentrations of sodium hexanoate starting substrate, plus Brij-35.

FIG. 20 shows cell titer and PHA titer (lines) and % PHA inclusion (markers) after 48 h of E. coli ptsG STJ4 shake flask growth with varying concentrations of sodium hexanoate starting substrate.

FIG. 21 shows PHB-co-PHHx comonomer ratios (bars) and final pH (line) after 48 h of E. coli ptsG STJ4 shake flask growth with varying concentrations of sodium hexanoate starting substrate.

FIG. 22 shows cell titer and PHA titer (lines) and % PHA inclusion (markers) after 48 h of E. coli ptsG STJ4 shake flask growth with varying concentrations of glucose addition at t=7 hours.

FIG. 23 shows PHB-co-PHD comonomer ratios (bars) and final pH (line) after 48 h of E. coli ptsG STJ4 shake flask growth with varying concentrations of glucose addition at t=7 hours.

### DETAILED DESCRIPTION OF THE INVENTION

The present disclosure provides one or more microorganisms and/or expression cassettes useful for biologically producing PHA homopolymers and/or PHA copolymers, including PHB-co-MCL copolymers of controllable or predetermined composition. In embodiments, the present disclosure provides a nucleic acid sequence or nucleic acid construct suitable for use in a microorganism and/or expression cassette. For example, in embodiments, a nucleic acid sequence or nucleic acid construct includes: one or more genes including a phaJ4 gene, a phaA gene, a phaB gene, a phaC1 gene, or combinations thereof; a cDNA that encodes one or more proteins including an enoyl-CoA hydratase 2, a β-ketothiolase, an acetoacetyl-CoA reductase, a type II polyhydroxyalkanoate synthase, or combinations thereof; or one or more nucleic acid sequences that encode one or more proteins including an enoyl-CoA hydratase 2, a β-ketothiolase, an acetoacetyl-CoA reductase, a type II polyhydroxyalkanoate synthase, or combinations thereof.

Embodiments of the present disclosure include one or more vectors, host cells and methods for controllable biosynthesis of a diverse array of polyhydroxybutyrate (PHB) based polyhydroxyalkanoate (PHA) copolymers in recombinant Escherichia coli. Non-limiting examples of Escherichia coli include E. coli LSBJ and recombinant Escherichia coli LSBJ Crp *. Advantages include providing for the diverse and controllable production of PHA homopolymers and copolymers from direct fatty acid precursors, and the production of poly((R)-3-hydroxybutyrate) (PHB) from non-fatty acid non-related precursors (previous work requires a butyric acid precursor to make the same product). Advantages of the present disclosure further include bioconverting a substrate such as a fatty acid precursor or carbon source directly to PHA homopolymers and copolymers using a single microorganism or strain thereof, and improved PHA homopolymers and copolymers yield at levels not previously obtained, or a preselected uniformity.

The disclosed host strains, including production host strains, have been engineered to maximize the substrate diversity and control production of PHA homopolymers and copolymers from direct fatty acid precursors, and the production of poly((R)-3-hydroxybutyrate) (PHB) from non-fatty acid non-related precursors. In embodiments, those transformations and mutations can be combined so as to control particular enzyme activities for the enhancement of PHA homopolymers and copolymers including poly((R)-3-hydroxybutyrate) (PHB) production. Thus, it is within the scope of the present disclosure to anticipate modifications of a whole cell catalyst which lead to an increased production of PHA homopolymers and copolymers including poly((R)-3-hydroxybutyrate) (PHB) production.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

The term “about”, as used herein, refers to +/−10% of the stated value or a chemical or obvious equivalent thereof.

“Binding” as used herein refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻¹¹ M, less than 10⁻¹² M, or less than 10⁻¹⁵ M. “Affinity” refers to the strength of binding, increased binding affinity being correlated with a lower Kd.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The terms “carbon substrate” and “carbon source” refer to a carbon source capable of being metabolized by host microorganisms of the present disclosure and particularly carbon sources including monosaccharides, oligosaccharides, polysaccharides, one-carbon substrates, or mixtures thereof.

The term “complementary deoxynucleotide” or “cDNA” means a DNA molecule that can be prepared by reverse transcription from a mature, spliced, mRNA molecule obtained from a cell, such as eukaryotic or prokaryotic cells. cDNA lacks introns or intron sequences that may be present in corresponding genomic DNA. In embodiments, cDNA may refer to a nucleotide sequence that correspond to the nucleotide sequence of an mRNA from which it is derived. In embodiments, cDNA refers to a double-stranded DNA that is complementary to and derived from mRNA.

The term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes. Complementarity is typically measured with respect to a duplex region and thus excludes, for example, overhangs. A duplex region may include a region of complementarity between two strands or between two regions of a single strand, for example, a unimolecular siRNA. Typically, the region of complementarity results from Watson-Crick base pairing. In embodiments, perfect complementarity or 100% complementarity refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other. For example, for two 20-mers, if only two base pairs on each strand can hydrogen bond with each other, the polynucleotide strands or regions exhibit 10% complementarity. In the same example, if 18 base pairs on each strand or each region can hydrogen bond with each other, the polynucleotide strands exhibit 90% complementarity. Substantial complementarity refers to polynucleotide strands or regions exhibiting 80% or greater complementarity.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Non-limiting exemplary conservative amino acid substitution groups are: valine-leucine-isoleucine, phenylalanine-tyrosine, lysine-arginine, alanine-valine-glycine, and asparaginc-glutaminc.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences.

The term “deoxynucleotide” refers to a nucleotide or polynucleotide lacking an OH group at the 2′ or 3′ position of a sugar moiety, and/or a 2′,3′ terminal dideoxy, but instead having a hydrogen at the 2′ and/or 3′ carbon.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

The terms “encoding” and “coding” refer to the process by which a gene, through the mechanisms of transcription and translation, produces an amino acid sequence. It is understood that the process of encoding a specific amino acid sequence includes DNA sequences that may involve base changes that do not cause a change in the encoded amino acid, or which involve base changes which may alter one or more amino acids, but do not affect the functional properties of the protein encoded by the DNA sequence. It is therefore understood that the invention encompasses more than the specific exemplary sequences.

The term “expression” refers to the transcription and translation to gene product from a gene coding for the sequence of the gene product.

The terms “foreign gene”, “foreign DNA”, “heterologous gene” and “heterologous DNA” refer to genetic material native to one organism that has been placed within a host microorganism by various methods. The gene of interest may be a naturally occurring gene, a mutated gene, or a synthetic gene.

“Gene” refers to a nucleic acid fragment that expresses a specific protein, including regulatory sequences preceding (5′ non-coding) and following (3′ non-coding) the coding region. The terms “native” and “wild-type” refer to a gene as found in nature with its own regulatory sequences.

The term “genetically altered” refers to the process of changing hereditary material by transformation or mutation.

The terms “host cell” or “host microorganism” refer to a microorganism capable of receiving foreign or heterologous genes and of expressing those genes to produce an active gene product.

By “hybridizable” or “complementary” or “substantially complementary” a nucleic acid (e.g., RNA, DNA) includes a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidinc/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e.., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

As used herein, the terms “isolated nucleic acid fragment”, and “isolated nucleic acid molecule” are used interchangeably and are optionally single-stranded or double-stranded with synthetic, non-natural or modified nucleotide bases. This will indicate a single-stranded RNA or DNA polymer.

The term “isolated” refers to a protein or DNA sequence that is removed from at least one component with which it is naturally associated.

The term “nucleotide” refers to a ribonucleotide or a deoxyribonucleotide or modified form thereof, as well as an analog thereof. Nucleotides include species that include purines, e.g., adenine, hypoxanthine, guanine, and their derivatives and analogs, as well as pyrimidines, e.g., cytosine, uracil, thymine, and their derivatives and analogs. In embodiments, a “nucleotide” includes a cytosine, uracil, thymine, adenine, or guanine moiety. In embodiments, nucleotides, unless otherwise specified (such as, for example, when specifying a 2′ modification, 5′ modification, 3′ modification, nucleobase modification, or modified internucleotide linkage), include unmodified cytosine, uracil, thymine, adenine, and guanine. In embodiments, nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′—OH is replaced by a group such as an H, OR, R, halo, SH, SR, NH2, NHR, NR2, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides. In embodiments, modified bases refer to nucleotide bases such as, for example, adenine, guanine, cytosine, thymine, and uracil, xanthine, inosine, and queuosine that have been modified by the replacement or addition of one or more atoms or groups. Some examples of types of modifications that can include nucleotides that are modified with respect to the base moieties, include but are not limited to, alkylated, halogenated, thiolated, aminated, amidated, or acetylated bases, in various combinations. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosinc, 5-methylaminocthyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archacosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridinc-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularinc. Further, the term nucleotide also includes those embodiments or species that have a detectable label, such as for example a radioactive or fluorescent moiety, or mass label attached to the nucleotide.

The phrase “nucleotide unit” refers to a single nucleotide residue and is includes a modified or unmodified nitrogenous base, a modified or unmodified sugar, and a modified or unmodified moiety that allows for linking of two nucleotides together or a conjugate that precludes further linkage.

As used herein, the term “nucleic acid molecule” refers to any molecule containing multiple nucleotides (i.e.., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single-or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

The terms “plasmid”, “vector”, and “cassette” refer to an extra chromosomal element often carrying genes which are not part of the central metabolism of a cell, and usually in the form of circular double-stranded DNA molecules. Such elements may be autonomously replicating sequences, genome integrating sequences, phage or nucleotide sequences, linear or circular, of a single-or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host. In embodiments, an “expression cassette” also includes a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. In embodiments, a “vector” or “expression vector” is a replicon, such as plasmid, phage, virus, or cosmid, to which another DNA segment, i.e., an “insert”, may be attached so as to bring about the replication of the attached segment in a cell.

The terms “recombinant microorganism” and “transformed host” refer to any microorganism having been transformed with heterologous or foreign genes or extra copies of homologous genes.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The terms “transformation” and “transfection” refer to the acquisition of new genes in a cell after the incorporation of nucleic acid. The acquired genes may be integrated into chromosomal DNA or introduced as extrachromosomal replicating sequences. The term “transformant” refers to the product of a transformation.

The terms “recombinant expression vector,” or “DNA construct” are used interchangeably herein to refer to a DNA molecule including a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “recombinant microorganism” and “transformed host” refer to any microorganism having been transformed with heterologous or foreign genes or extra copies of homologous genes. The recombinant microorganisms may include one or more E. coli cells including a disrupted endogenous β-oxidation pathway including a genetically disrupted endogenous fadB gene and/or a genetically disrupted fadJ gene. In embodiments, the recombinant microorganisms of the present disclosure express one or more foreign genes that encode one or more proteins including an enoyl-CoA hydratase 2, a β-ketothiolase, an acetoacetyl-CoA reductase, a type II polyhydroxyalkanoate synthase, or combinations thereof, for the production of polyhydroxybutyrate (PHB) based polyhydroxyalkanoate (PHA) copolymers from one or more suitable substrates. An embodiment is an E. coli transformed with one or more plasmids including these genes or cDNA thereof, wherein the E. coli lacks a functional fadB gene and/or fadJ gene.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology. “Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

In embodiments, the present disclosure includes a nucleic acid construct or nucleic acid molecule including: one or more genes including a phaJ4 gene, a phaA gene, a phaB gene, a phaC1 gene, or combinations thereof; a cDNA that encodes one or more proteins including an enoyl-CoA hydratase 2, a β-ketothiolase, an acetoacetyl-CoA reductase, a type II polyhydroxyalkanoate synthase, or combinations thereof; or one or more nucleic acid sequences that encode one or more proteins including an enoyl-CoA hydratase 2, a β-ketothiolase, an acetoacetyl-CoA reductase, a type II polyhydroxyalkanoate synthase, or combinations thereof.

In embodiments, one or more genes including a phaJ4 gene, include one or more genes suitable for the expression of enoyl-CoA hydratase 2 activity in a host cell. Genes encoding enoyl-CoA hydratase 2 activity are known. For example, enoyl-CoA hydratase 2 has been isolated from Arabidopsis thaliana. For the purposes of the present disclosure, it is contemplated that any gene encoding a polypeptide responsible for enoyl-CoA hydratase 2 activity is suitable where the activity is capable of degrading cis-unsaturated fatty acids, or degrading enoyl-CoA to (R)-3-hydroxyacyl-CoA.

In embodiments, one or more genes including a phaA gene, include one or more genes suitable for the expression of a β-ketothiolase activity in a host cell. Genes encoding a β-ketothiolase activity are known. For the purposes of the present disclosure, it is contemplated that any gene encoding a polypeptide responsible for a β-ketothiolase activity is suitable where the activity is capable condensing two acetyl-CoA units to form acetoacetyl-CoA. Alternatively, the β-ketothiolase activity is suitable where the activity is capable of condensing a propionyl-CoA and an acetyl-CoA to form acetopropionyl-CoA.

In embodiments, one or more genes, including a phaB gene, include one or more genes suitable for the expression of an acetoacetyl-CoA reductase activity in a host cell. Genes encoding a acetoacetyl-CoA reductase activity are known. For the purposes of the present disclosure, it is contemplated that any gene encoding a polypeptide responsible for a acetoacetyl-CoA reductase activity is suitable where the activity is capable of reducing acetoacetyl-CoA to (R)-3-hydroxybutyryl-CoA when reacted with NADPH. Alternatively, the acetoacetyl-CoA reductase activity is suitable where the activity is capable of reducing acctopropionyl-CoA to (R)-3-hydroxyvaleryl-CoA when reacted with NADPH. See e.g., BRENDA database EC 1.1.1.36.

In embodiments, one or more genes including a phaC1 gene, include one or more genes suitable for the expression of a type II polyhydroxyalkanoate synthase activity in a host cell. Genes encoding an engineered broad-specificity type II polyhydroxyalkanoate synthase activity are known. For the purposes of the present disclosure, it is contemplated that any gene encoding a polypeptide responsible for a engineered broad-specificity type II polyhydroxyalkanoate synthase activity is suitable where the activity is capable of forming PHB-co-MCL copolymer when reacted with (R)-3-hydroxybutryl-CoA and (R)-3-hydroxyacyl-CoA.

In embodiments, a nucleic acid construct suitable for use herein includes a plurality of genes including a phaJ4 gene, a phaA gene, a phaB gene, a phaC1 gene. In embodiments, the genes are recombinantly assembled into a nucleic acid construct including predetermine genes, or a predetermined plurality of genes using methods known in the art. Procedures for phosphorylations, ligations and transformations are well known in the art. Techniques suitable for use herein may be found in Sambrook, J., Fritsch, E. F. and Maniatis, T. Molecular Cloning: A Laboratory Manual, Second Edition, Cold Spring Harbor Laboratory Press, Cold Spring Harbor (1989). Non-limiting example of a DNA constructs for use herein include the DNA sequence identified as SEQ ID NO: 3, or substantially similar DNA sequences. In embodiments, suitable DNA constructs for use herein includes sequences having at least 85%, at least 90%, at least 95%, at least 97%, or at least 99% sequence identity to SEQ ID NO: 3.

In some embodiments, the present disclosure provides a nucleic acid construct suitable for use in a microorganism and/or expression cassette including a nucleic acid construct including a cDNA that encodes one or more proteins including an enoyl-CoA hydratase 2, a β-ketothiolase, an acetoacetyl-CoA reductase, a type II polyhydroxyalkanoate synthase, or combinations thereof. In embodiments, cDNA is synthesized via reverse transcription from an RNA template using techniques known in the art. Primers and protocols for reverse transcription are known in the art. See e.g., www.neb.com/applications/cloning-and-synthetic-biology/dna-preparation/reverse-transcription-cdna-synthesis.

In embodiments, the present disclosure provides one or more nucleic acid constructs suitable for use in a microorganism and/or one or more expression cassettes including one or more nucleic acid sequences that encode one or more proteins including an enoyl-CoA hydratase 2, β-ketothiolasc, acetoacetyl-CoA reductase, type II polyhydroxyalkanoate synthase, or combinations thereof. In embodiments, enoyl-CoA hydratase 2, β-ketothiolase, acetoacetyl-CoA reductase, type II polyhydroxyalkanoate synthase include those described in the publicly available enzyme database. Sec e.g., BRENDA:EC4.2.1.119, BRENDA:EC1.14.14.54, BRENDA:EC1.1.1.36, and cBRENDA:EC2.3.1.B3, respectively. BRENDA is available at www.brenda-enzymes.org.

In embodiments, the enzymes of the present disclosure, such as enoyl-CoA hydratase 2, a β-ketothiolase, an acetoacetyl-CoA reductase, a type II polyhydroxyalkanoate synthase, have activity suitable to facilitate an engineering metabolic pathway for the production of PHB-co-MCL PHA copolymers, for example in E. coli, such as E. coli LSBJ, harboring one or more plasmids (such as pBBRSTQKJ4) of the present disclosure.

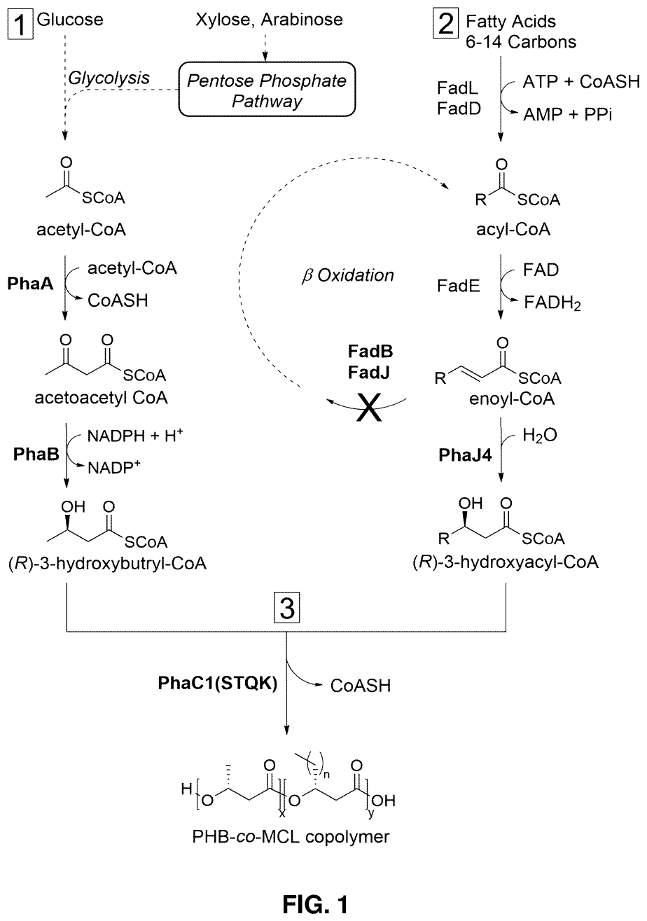

Referring now to FIG. 1 at (1), E. coli is able to naturally uptake hexose and pentose sugars glucose, xylose and arabinose, which are converted through glycolysis and the pentose phosphate pathway to acetyl-CoA. C. necator derived ketothiolase (PhaA) and acetoacetyl reductase (PhaB) convert acetyl-CoA to (R)-3-hydroxybutyryl-CoA intermediates. Referring to FIG. 1 at (2), a mutated fadR gene in E. coli LSBJ allows for constitutive (without induction) expression of fad genes responsible for uptake and conversion of fatty acid substrates to enoyl-CoA intermediates (FadL, FadD, FadE). Deletion of genes coding for FadB and FadJ prevents enoyl-CoA intermediates from completing β-oxidation, and therefore preserves the carbon number of the original fatty acid. A Pseudomonas putida KT2440 enoyl-CoA hydratase (PhaJ4) converts enoyl-CoA to the (R)-3-hydroxyacyl-CoA intermediate. Referring to FIG. 1 at (3) A Pseudomonas sp. 61-3 PHA synthase (PhaC1(STQK)) engineered for broad substrate uptake incorporates the acyl- and butyryl-CoA intermediates into the growing PHA copolymer. In embodiments, the process sequence of FIG. 1 ends with the production of PHB-co-MCL copolymer. In embodiments, the PHB-co-MCL copolymer is characterized as uniform having over 80%, over 90%, over 95% uniformity on structure or carbon numbers. Non-limiting examples of carbon numbers include 4-14 carbons.

### Vectors and Expression Cassettes

The present disclosure provides a variety of vectors and transformation and expression cassettes suitable for the cloning, transformation and expression of proteins including one or more of an enoyl-CoA hydratase 2, a β-ketothiolase, an acetoacetyl-CoA reductase, a type II polyhydroxyalkanoate synthase into a suitable host cell. Suitable vectors will be those which are compatible with the microorganism employed. In embodiments suitable vectors can be derived, for example, from a bacterium, a virus (such as bacteriophage T7 or a M-13 derived phage), a cosmid, a yeast or a plant. Protocols for obtaining and using such vectors are known to those in the art (Sambrook et al., supra).

Initiation control regions, or promoters, which are useful to drive expression of the phaJ4 gene, phaA gene, phaB gene, and/or phaC1 genes in the desired host cell are numerous and familiar to those skilled in the art. Virtually any promoter capable of driving these genes is suitable for the present disclosure including but not limited to lac, trp, λPL, λPR, T7, tac, and trc useful for expression in E. coli.

Termination control regions may also be derived from various genes native to the preferred hosts. Optionally, a termination site may be unnecessary; in embodiments, is included. In some embodiments, for effective expression of the instant enzymes, DNA encoding the enzymes are linked operably through initiation codons to selected expression control regions such that expression results in the formation of the appropriate messenger RNA.

In some embodiments, the present disclosure relates to an expression vector including: a) a promoter sequence; and b) a nucleic acid construct including: one or more genes including a phaJ4 gene, a phaA gene, a phaB gene, a phaC1 gene, or combinations thereof; a cDNA that encodes one or more proteins comprising an enoyl-CoA hydratase 2, a β-ketothiolase, an acetoacetyl-CoA reductase, a type II polyhydroxyalkanoate synthase, or combinations thereof; or one or more nucleic acid sequences that encode one or more proteins comprising an enoyl-CoA hydratase 2, a β-ketothiolasc, an acetoacetyl-CoA reductase, a type II polyhydroxyalkanoate synthase, or combinations thereof.

Particularly useful in the present disclosure is the pBBR1 vector. See e.g., FIGS. 6 showing a plasmid in accordance with the present disclosure depicting the replication origin for the pBBR1 vector.

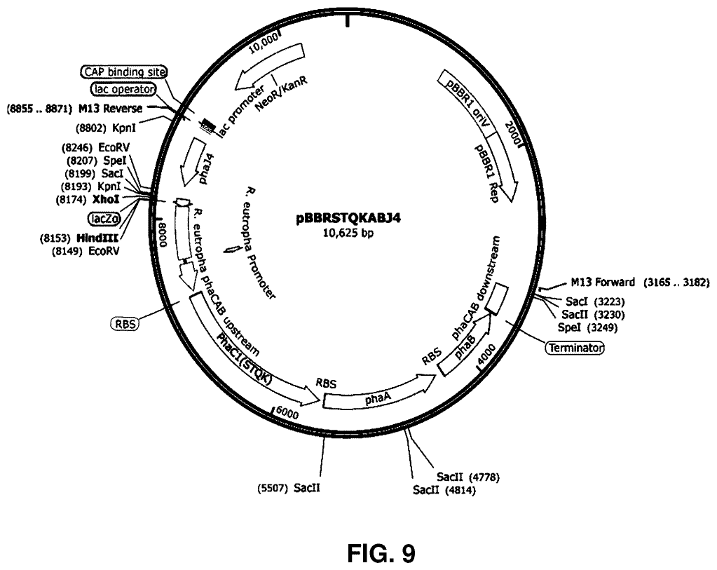

FIG. 9 depicts a plasmid map of pBBRSTQKABJ4 displaying location and expression control mechanisms of phaJ4, PhaC1, PhaA, and PhaB in accordance with the present disclosure. pBBR1 oriV (SEQ ID NO: 4): Replication origin for the pBBR1 vector, requires pBBR1 Rep protein pBBR1 Rep (SEQ ID NO: 5): Codes for the replication protein for the pBBR1 vector. phaCAB downstream: 3′ non-coding region from the C. necator phaCAB operon.

phaB (SEQ ID NO: 6): Codes for C. necator PhaB acetoacetyl-CoA reductase.
phaA (SEQ ID NO: 7): Codes for C. necator PhaA β-ketothiolase.
phaC1 (STQK) (SEQ ID NO: 8): Codes for a Pseudomonas sp. 61-3 PhaC PHA synthase, which has been modified for enhanced uptake of SCL acyl-CoA substrates.
R. eutropha phaCAB upstream: 5′ non-coding region from the C. necator phaCAB operon which contains the C. necator promoter.
R. eutropha promoter (SEQ ID NO: 9): A constitutive promoter from C. necator that recruits RNA polymerase to begin transcription. lacZa: The lacZα fragment of β-galactosidase (a relic of blue-white screening).
phaJ4 (SEQ ID NO: 10): Codes for Pseudomonas putida PhaJ4 (R)-specific enoyl-CoA hydratase. lac operator: Prevents RNA polymerase from binding to the plasmid, and is negatively controlled by isopropylthiogalactosidase (IPTG) to begin transcription of the operon.
lac promoter: Recruits RNA polymerase to begin transcription.
CAP binding site: Binding site for catabolite activator protein, which activates transcription in the presence of cAMP.
NeoR/KanR: Codes for resistance to kanamycin antibiotic, for strain selection and culture maintenance purposes.

### Host Cells

Suitable host cells for the recombinant production of PHA homopolymers and/or PHA copolymers, including PHB-co-MCL copolymers of controllable or predetermined composition may be either prokaryotic or eukaryotic and will be limited only by the host cell ability to express the active enzymes for the set forth in the FIG. 1 pathway. Suitable host cells will be microorganisms such as Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. In embodiments, suitable host cells include Escherichia coli, Escherichia blattae, Klebsiella, Citrobacter, and Aerobacter. In embodiments a suitable host cell for use herein includes E. coli. In some embodiments, the host cells of the present disclosure include recombinant Escherichia coli LSBJ or recombinant Escherichia coli LSBJ Crp* harboring multiple genes for PHA biosynthesis via transformation with the plasmid STJ4. In embodiments, the host cells of the present disclosure are characterized as or include: 1) recombinant E. coli having had fatty acid β-Oxidation interrupted by disabling production of the enzymes FadB and FadJ, which causes a build-up of enoyl-CoA intermediates; 2) the plasmid STJ4 providing for production of the (R)-specific enoyl-CoA hydratase PhaJ4, which converts enoyl-CoA intermediates to (R)-3-hydroxyacyl-CoA equivalents; 3) the plasmid STJ4 providing for production of the enzymes PhaA and PhaB, which condense 2 acetyl-CoAs to (R)-3-hydroxybutyryl-CoA; and 4) the plasmid STJ4 providing for production of the engineered type II two PHA synthase PhaC1, which can use all products from parts 2) and 3) as substrates for polymerization into PHA.

In embodiments, the combination of E. coli LSBJ or E. coli LSBJ Crp* with the plasmid STJ4 allows for simultaneous biosynthesis of PHB from non-fatty acid nonrelated resources (glucose, glycerol, lactic acid, etc.) and production of medium chain length PHAs (MCL-PHA) from fatty acid precursors up to 14 carbons long. In embodiments, the simultaneous biosynthetic pathways allow for predictable, controllable production of PHB-co-MCL PHAs which can partially originate from a diverse array of non-fatty acid substrates. The PHB-co-MCL copolymers produced can be made of varied, predictable composition, where the composition can be dictated by the mass ratio of substrates supplied to the E. coli organism. The MCL copolymer portion can be any of a single or combination of (R)-3-hydroxyacyl-CoA intermediates up to 14 carbons long. PHAs of differing copolymer composition have differing physical properties, which allows for production of a huge array of PHA polymers with a greater number of uses.

In embodiments, the host cells of the present disclosure include an E. coli cell including a disrupted endogenous β-oxidation pathway including a genetically disrupted endogenous fadB gene and/or a genetically disrupted fadJ gene, and a nucleic acid construct including a phaJ4 gene encoding active enoyl-CoA hydratase 2, at least one phaA gene encoding active β-ketothiolase, at least one phaB gene encoding active acetoacetyl-CoA reductase, and at least one phaC1 (STQK) gene encoding active type II polyhydroxyalkanoate synthase. In embodiments, the nucleic acid construct includes a Cupriavidus necator constitutive promoter operatively linked to the nucleic acid construct. In embodiments, the Cupriavidus necator constitutive promoter is operatively linked to one or more nucleic acids comprising: a phaA gene encoding active β-ketothiolase, a phaB gene encoding active acetoacetyl-CoA reductase, and a phaC1 gene encoding active type II polyhydroxyalkanoate synthase, or ccDNA encoding one or more proteins comprising a β-ketothiolase, a acetoacetyl-CoA reductase, a type II polyhydroxyalkanoate synthase, or combinations thereof. In embodiments, host cell includes one or more copies of the nucleic acid construct, such as 5-10 copies of the nucleic acid construct. In embodiments, the nucleic acid construct further includes a selectable marker gene. In embodiments, the nucleic acid construct further includes no foreign selectable marker gene. In embodiments, one or more coding sequences involved in a biosynthesis of PHA homopolymers and/or PHA copolymers are selected from the group consisting of a phaJ4 gene, a phaA gene, a phaB gene, and a phaC1 gene, or are selected from the group consisting of a cDNA sequences of a phaJ4 gene, a cDNA sequences of phaA gene, a cDNA sequences of a phaB gene, or cDNA sequences of phaC1 gene. In embodiments, the nucleic acid construct is contained on an extrachromosomal element.

In some embodiments, the present disclosure includes an E. coli strain comprising: i) a disrupted endogenous β-oxidation pathway comprising a genetically disrupted endogenous fadB gene and/or a genetically disrupted fadJ gene resulting in a buildup of one or more enoyl-CoA intermediates; ii) a genetically upregulated phaJ4 gene encoding active enoyl-CoA hydratase 2 with the upregulation resulting in an increased (R)-3-hydroxyacyl-CoA equivalents, wherein the upregulation is produced by introducing at least one phaJ4 gene into the E. coli strain; iii) a genetically upregulated phaA gene encoding active β-ketothiolase the upregulation resulting in an increased (R)-3-hydroxybutyryl-CoA, wherein the upregulation is produced by introducing at least one phaA gene into the E. coli strain; iv) a genetically upregulated phaB gene encoding active acetoacetyl-CoA reductase the upregulation resulting in an increased (R)-3-hydroxybutyryl-CoA, wherein the upregulation is produced by introducing at least one phaB gene into the E. coli strain; and v) a genetically upregulated phaC1 gene encoding active type II polyhydroxyalkanoate synthase, the upregulation resulting in an increased PHA, wherein the upregulation is produced by introducing at least one phaC1 gene into the E. coli strain, wherein said E. coli strain is capable of bioconverting a suitable substrate to PHA homopolymers and/or PHA copolymers. In embodiments, the active enoyl-CoA hydratase 2 converts one or more enoyl-CoA intermediates to one or more (R)-3-hydroxyacyl-CoA equivalents. In embodiments, the active enoyl-CoA hydratase 2 is (R)-specific enoyl-CoA hydratase PhaJ4. In embodiments, the active β-ketothiolase and active acetoacetyl-CoA reductase condense 2 acetyl-CoAs to (R)-3-hydroxybutyryl-CoA. In embodiments type two PHA synthase PhaC1 converts one or more (R)-3-hydroxyacyl-CoA equivalents and (R)-3-hydroxybutyryl-CoA into PHA or PHB-co-MCL PHA copolymers. In embodiments, the E. coli strain is characterized as not having the capability to degrade PHAs.

### Media and Carbon Substrates

Fermentation media in the present invention must contain suitable carbon substrates. Suitable substrates may include but are not limited to monosaccharides such as glucose and fructose and oligosaccharides such as lactose or sucrose. In embodiments, a suitable carbon substrate is glucose.

### Culture Conditions

Typically, cells are grown at 35° C. in appropriate media. Growth media suitable for use herein are common commercially prepared media such as Luria Bertani (LB) broth, Sabouraud Dextrose (SD) broth or Yeast medium (YM) broth. Other defined or synthetic growth media may also be used and the appropriate medium for growth of the particular microorganism will be known by someone skilled in the art of microbiology or fermentation science. The use of agents known to modulate catabolite repression directly or indirectly, e.g., cyclic adenosine 2′: 3′-monophosphate, may also be incorporated into the reaction media. Similarly, the use of agents known to modulate enzymatic activities (e.g., methyl viologen) that lead to enhancement of production may be used in conjunction with or as an alternative to genetic manipulations.

Suitable pH ranges for the fermentation are between pH 5.0 to pH 9.0, where pH 6.0 to pH 8.0 is preferred as the initial condition. Reactions may be performed under aerobic or anaerobic conditions where acerobic, anoxic, or anaerobic conditions are preferred based on the requirements of the microorganism. Fed-batch fermentations may be performed with carbon feed, for example, glucose, limited or excess.

### Batch and Continuous Fermentations

The present process employs a batch method of fermentation. Classical batch fermentation is a closed system where the composition of the media is set at the beginning of the fermentation and is not subject to artificial alterations during the fermentation. Thus, at the beginning of the fermentation the media is inoculated with the desired microorganism or microorganisms and fermentation is permitted to occur adding nothing to the system. Typically, however, “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present disclosure and include a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen, and the partial pressure of waste gases such as CO2. Batch and Fed-Batch fermentations are common and well known in the art and examples may be found in Brock, supra.

In embodiments, the present disclose is performed in batch mode, however it is contemplated that the method would be adaptable to continuous fermentation methods. Continuous fermentation is an open system where a defined fermentation media is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present disclosure may be practiced using batch, fed-batch or continuous processes and that any known mode of fermentation would be suitable. Additionally, it is contemplated that cells may be immobilized on a substrate as whole cell catalysts and subjected to fermentation conditions for polymer production.

Identification and Purification of PHA homopolymers and/or PHA copolymers, including PHB-co-MCL copolymers of controllable or predetermined composition

Methods for the purification of PHA homopolymers and/or PHA copolymers, including PHB-co-MCL copolymers of controllable or predetermined composition from fermentation media are known in the art. For example, PHA homopolymers and/or PHA copolymers, including PHB-co-MCL copolymers of controllable or predetermined composition can be obtained from cell media by subjecting the reaction mixture to filtration to separate cells from the media, or centrifugation to separate cells from the media, followed by extraction of the PHA with an organic solvent, or extraction of cell debris with surfactant and chelating agent in basic aqueous solvent.

PHA homopolymers and/or PHA copolymers, including PHB-co-MCL copolymers of controllable or predetermined composition may be identified directly by submitting the isolated PHA material to gas chromatography (GC), or to nuclear-magnetic resonance spectroscopy (NMR).

### General Methods and Materials

Procedures for phosphorylations, ligations and transformations are well known in the art. Techniques suitable for use in the following examples may be found in Sambrook, J. et al., supra.

Materials and methods suitable for the maintenance and growth of bacterial cultures are well known in the art. Techniques suitable for use in the following examples may be found in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994) or Thomas D. Brock in Biotechnology: A Textbook of Industrial Microbiology, Second Edition (1989) Sinauer Associates, Inc., Sunderland, Mass. All reagents and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Strains and vectors used and constructed in the following examples are described further in Tables 1 and 2 in the Examples below.

In embodiments, the present disclosure includes one or more nucleic acid constructs or nucleic acid molecules including: one or more genes comprising a phaJ4 gene, a phaA gene, a phaB gene, a phaC1 gene, or combinations thereof; a cDNA that encodes one or more proteins comprising an enoyl-CoA hydratase 2, a β-ketothiolase, an acetoacetyl-CoA reductase, a type II polyhydroxyalkanoate synthase, or combinations thereof; or one or more nucleic acid sequences that encode one or more proteins comprising an enoyl-CoA hydratase 2, a β-ketothiolase, an acetoacetyl-CoA reductase, a type II polyhydroxyalkanoate synthase, or combinations thereof.

In embodiments, the present disclosure includes an expression vector including: a) a promoter sequence; and b) a nucleic acid construct including: one or more genes comprising a phaJ4 gene, a phaA gene, a phaB gene, a phaC1 gene, or combinations thereof; a cDNA that encodes one or more proteins comprising an enoyl-CoA hydratase 2, a β-ketothiolase, an acetoacetyl-CoA reductase, a type II polyhydroxyalkanoate synthase, or combinations thereof; or one or more nucleic acid sequences that encode one or more proteins comprising an enoyl-CoA hydratase 2, a β-ketothiolase, an acetoacetyl-CoA reductase, a type II polyhydroxyalkanoate synthase, or combinations thereof. In embodiments, the present disclosure includes a promoter sequence is operably linked to the nucleic acid construct or nucleic acid molecule. In embodiments, the promoter sequence is characterized as Ralstonia eutropha promoter, or a Cupriavidus necator constitutive promoter. In embodiments, the expression vector further comprises a selectable marker gene. In embodiments, the expression vector further includes no foreign selectable marker gene. In embodiments, the one or more genes comprise one or more coding sequences involved in a biosynthesis of PHA homopolymers and/or PHA copolymers. In embodiments, the nucleic acid construct is contained on an extrachromosomal element. In embodiments, the expression vector is a plasmid. In embodiments, the present disclosure includes a host cell including the expression vector of the present disclosure. In embodiments, the host cell is Escherichia coli bacterium.

In some embodiments, the present disclosure includes a method of making PHA homopolymers and/or PHA copolymers, including: (a) culturing a host cell of claim 10 in a culture medium under conditions suitable for bioconverting a substrate to PHA homopolymers and/or PHA copolymers; and (b) isolating PHA or PHA copolymer from the culture medium or from the host cell. In embodiments, the substrate incudes one or more predetermined substrates. In embodiments, the substrate includes fatty acid substrates, fatty acid substrates comprising up to 14 carbons, hexose sugar, pentose sugar, glucose, xylose, arabinose, mannose, galactose, fatty acids comprising 6-14 carbons, and combinations thereof.

In some embodiments the present disclosure includes a method of forming polyhydroxyalkanoate (PHA) homopolymers and/or PHA copolymers, including: cultivating a recombinant E. coli cell in a medium conducive for a production of the polyhydroxyalkanoate (PHA) homopolymers and/or PHA copolymers, wherein the recombinant E. coli cell comprises a disrupted endogenous β-oxidation pathway comprising a genetically disrupted endogenous fadB gene and/or a genetically disrupted fadJ gene, and a nucleic acid construct comprising a phaJ4 gene, cDNA, or nucleic acid encoding active enoyl-CoA hydratase 2, at least one phaA gene, cDNA, or nucleic acid encoding active β-ketothiolase, at least one phaB gene, cDNA, or nucleic acid encoding active acetoacetyl-CoA reductase, and at least one phaC1 gene, cDNA, or nucleic acid encoding active type II polyhydroxyalkanoate synthase, wherein cultivating occurs under conditions suitable for bioconverting a substrate to PHA homopolymers and/or PHA copolymers. In embodiments, the substrate is characterized as predetermined. In embodiments, the substrate includes one or more of fatty acid substrates, fatty acid substrates comprising up to 14 carbons, hexose sugar, pentose sugar, glucose, xylose, arabinose, mannose, galactose, fatty acids comprising 6-14 carbons, and combinations thereof. In embodiments, the substrate is derived from lignocellulosic waste, municipal waste, or fatty acid waste, or palm kernel fatty acid distillate (such as pkfad).

In embodiments, the recombinant E. coli cell contains at least one copy of the nucleic acid construct, such as for example a copy number of 1-10, 2-7, or at least one. In embodiments, the nucleic acid construct further comprises a selectable marker gene. In embodiments, the nucleic acid construct further comprises no foreign selectable marker gene. In embodiments, one or more coding sequences involved in a biosynthesis of PHA homopolymers and/or PHA copolymers are selected from the group consisting of a phaJ4 gene, phaA gene, phaB gene, phaC1 gene, and combinations thereof. In embodiments, the nucleic acid construct is contained on an extrachromosomal element.

In some embodiments, the present disclosure includes one or more E. coli. cells including a disrupted endogenous β-oxidation pathway comprising a genetically disrupted endogenous fadB gene and/or a genetically disrupted fadJ gene, and a nucleic acid construct comprising a phaJ4 gene encoding active enoyl-CoA hydratase 2, at least one phaA gene encoding active β-ketothiolase, at least one phaB gene encoding active acetoacetyl-CoA reductase, and at least one phaC1 (STQK) gene encoding active type II polyhydroxyalkanoate synthase. In embodiments, the nucleic acid construct comprises a Cupriavidus necator constitutive promoter operatively linked to the nucleic acid construct. In embodiments, the Cupriavidus necator constitutive promoter is operatively linked to one or more nucleic acids comprising: a phaA gene encoding active β-ketothiolase, a phaB gene encoding active acetoacetyl-CoA reductase, and a phaC1 gene encoding active type II polyhydroxyalkanoate synthase, or a cDNA encoding one or more proteins comprising a β-ketothiolase, an acetoacetyl-CoA reductase, a type II polyhydroxyalkanoate synthase, or combinations thereof. In embodiments, the cell comprises one or more copies of the nucleic acid construct, such as 1-10 copies. In embodiments, the nucleic acid construct further comprises a selectable marker gene, and in some embodiments, the nucleic acid construct further comprises no foreign selectable marker gene. In some embodiments, one or more coding sequences involved in a biosynthesis of PHA homopolymers and/or PHA copolymers are selected from the group consisting of a phaJ4 gene, a phaA gene, a phaB gene, and a phaC1 gene, or are selected from the group consisting of a cDNA sequences of a phaJ4 gene, a cDNA sequences of phaA gene, a cDNA sequences of a phaB gene, or a cDNA sequences of phaC1 gene. In some embodiments, the nucleic acid construct is contained on an extrachromosomal element.

In some embodiments, the present disclosure includes an E. coli strain comprising: i) a disrupted endogenous β-oxidation pathway comprising a genetically disrupted endogenous fadB gene and/or a genetically disrupted fadJ gene resulting in a buildup of one or more enoyl-CoA intermediates; ii) a genetically upregulated phaJ4 gene encoding active enoyl-CoA hydratase 2 with the upregulation resulting in an increased production of (R)-3-hydroxyacyl-CoA equivalents, wherein the upregulation is produced by introducing at least one phaJ4 gene into the E. coli strain; iii) a genetically upregulated phaA gene encoding active β-ketothiolase the upregulation resulting in an increased (R)-3-hydroxybutyryl-CoA, wherein the upregulation is produced by introducing at least one phaA gene into the E. coli strain; iv) a genetically upregulated phaB gene encoding active acetoacetyl-CoA reductase the upregulation resulting in an increased (R)-3-hydroxybutyryl-CoA, wherein the upregulation is produced by introducing at least one phaB gene into the E. coli strain; and v) a genetically upregulated phaC1 gene encoding active type II polyhydroxyalkanoate synthase, the upregulation resulting in an increased PHA, wherein the upregulation is produced by introducing at least one phaC1 gene into the E. coli strain, wherein said E. coli strain is capable of bioconverting a suitable substrate to PHA homopolymers and/or PHA copolymers. In embodiments, the active enoyl-CoA hydratase 2 converts one or more enoyl-CoA intermediates to one or more (R)-3-hydroxyacyl-CoA equivalents. In embodiments, the active enoyl-CoA hydratase 2 is R-specific enoyl-CoA hydratase PhaJ4. In embodiments, the active β-ketothiolase and active acetoacetyl-CoA reductase condense 2 acetyl-CoAs to (R)-3-hydroxybutyryl-CoA. In embodiments, two PHA synthase PhaC1 converts one or more (R)-3-hydroxyacyl-CoA equivalents and (R)-3-hydroxybutyryl-CoA into PHA or PHB-co-MCL PHA copolymers. In embodiments, the E. coli strain is characterized as not having the capability to degrade PHAs.

### Example 1

The present disclosure demonstrates a one-pot production of PHB, pure MCL PHA, and PHB-co-MCL block copolymer in E. coli by co-feeding of brewer's spent grain (BSG) enzymatic hydrolysate and sodium decanoate. This was accomplished by construction and expression of the plasmid pBBRSTQKABJ4 in β-oxidation fadB/fadJ disabled E. coli LSBJ, which was used to ferment the hydrolysate and fatty acid in shake flasks. Batch mode fermentation of BSG hydrolysate and sodium decanoate yielded a mixture of PHB homopolymer, PHD homopolymer, and PHB-co-PHD block copolymer at maximum PHA titers of 3.7 g/L, with the PHB/PHD ratio tunable according to substrate addition. Higher titers of PHA were unable to be achieved with increasing carbon supply due to acetate overflow manifesting toxic culture conditions.

Polyhydroxyalkanoates (PHA) are biodegradable polymers suitable as a replacement for significant quantities of non-biodegradable petroleum-based plastics in packaging. However, the wide-spread industrial implementation of PHA plastics has been impeded by high production costs, up to 50% of which can be accounted for in raw material sourcing (See e.g., P. Lhamo, S. K. Bchera, and B. Mahanty, “Process optimization, metabolic engineering interventions and commercialization of microbial polyhydroxyalkanoates production—A state-of-the art review,” Biotechnol. J., p. 2100136, June 2021, doi: 10.1002/biot.202100136).

Current commercial PHA production methods rely on dedicated agricultural crops for their carbon source, which is costly and environmentally unsustainable (See e.g., J. Brizga, K. Hubacek, and K. Feng, “The Unintended Side Effects of Bioplastics: Carbon, Land, and Water Footprints,” One Earth, vol. 3, no. 1, pp. 45-53, July 2020, doi: 10.1016/j.onccar.2020.06.016; and X. Zhao, K. Cornish, and Y. Vodovotz, “Narrowing the Gap for Bioplastic Use in Food Packaging: An Update,” Environ. Sci. Technol., vol. 54, no. 8, pp. 4712-4732 April 2020, doi: 10.1021/acs.est.9b03755). The use of waste materials and process side-streams as a basis for PHA production can greatly reduce cost and improve process sustainability (See e.g., A. Nayak and B. Bhushan, “An overview of the recent trends on the waste valorization techniques for food wastes,” J. Environ. Manage., vol. 233, pp. 352-370, March 2019, doi: 10.1016/j.jenvman.2018.12.041; and A. K. H. N. Aslan, M. D. M. Ali, N. A. Morad, and P. Tamunaidu, “Polyhydroxyalkanoates production from waste biomass,” IOP Conf. Ser. Earth Environ. Sci., vol. 36, p. 012040, June 2016, doi: 10.1088/1755-1315/36/1/012040).

Methods to enhance the properties of PHA are also being investigated in order to increase the potential of commercial applications for the biopolymer (See e.g., V. Sharma, R. Schgal, and R. Gupta, “Polyhydroxyalkanoate (PHA): Properties and Modifications,” Polymer, vol. 212, p. 123161, January 2021, doi: 10.1016/j.polymer.2020.123161). PHAs can be categorized as being short-chain length (3-5 carbons, SCL) or medium-chain length (6-14 carbons, MCL). Most natural PHA producers in typical conditions will either produce highly crystalline and brittle SCL PHA, or a very soft and flexible mixture of MCL PHA, both of which are undesirable for commercial applications. Combinations of SCL and MCL PHA, as blends or copolymers, can yield polymers with improved crystallinity, melting temperature, Young's modulus and flexural modulus (Sec e.g., I. Noda, P. R. Green, M. M. Satkowski, and L. A. Schechtman, “Preparation and Properties of a Novel Class of Polyhydroxyalkanoate Copolymers,” Biomacromolecules, vol. 6, no. 2, pp. 580-586, March 2005, doi: 10.1021/bm049472m; and R. D. Ashby, D. K. Y. Solaiman, and T. A. Foglia, “Synthesis of Short-/Medium-Chain-Length Poly (hydroxyalkanoate) Blends by Mixed Culture Fermentation of Glycerol,” Biomacromolecules, vol. 6, no. 4, pp. 2106-2112 July 2005, doi: 10.1021/bm058005h).

There have been great advances biosynthesis technologies of PHA copolymers in recent years. The most widely studied and industrially relevant PHA, Nodax™ by Danimer Scientific, is a tunable copolymer of PHB-co-PHHx produced by a recombinant strain of Cupriavidus necator grown on canola oil (See e.g., I. Noda, P. R. Green, M. M. Satkowski, and L. A. Schechtman, “Preparation and Properties of a Novel Class of Polyhydroxyalkanoate Copolymers,” Biomacromolecules, vol. 6, no. 2, pp. 580-586, March 2005, doi: 10.1021/bm049472m; and I. Noda, S. B. Lindsey, and D. Caraway, “Nodax™ Class PHA Copolymers: Their Properties and Applications,” in Plastics from Bacteria, vol. 14, G. G.-Q. Chen, Ed. Berlin, Heidelberg: Springer Berlin Heidelberg, 2010, pp. 237-255. doi: 10.1007/978-3-642-03287-5_10).

Pseudomonas entomophila was recently engineered to produce PHB-co-MCL copolymers with composition of the MCL fraction directly determinable from the fatty acid feed (See e.g., M. Li et al., “Engineering Pseudomonas entomophila for synthesis of copolymers with defined fractions of 3-hydroxybutyrate and medium-chain-length 3-hydroxyalkanoates,” Metab. Eng., vol. 52, pp. 253-262, March 2019, doi: 10.1016/j.ymben.2018.12.007). E. coli is an especially viable candidate for scalable recombinant production of PHA because it is adaptable to a large variety of substrates, high-density fermentation techniques are well established for it, it is relatively non-pathogenic compared to other production strains, and is a well-established expression vector for heterologous proteins. E. coli has been modified to express a variety of PHA production pathways, including Bacillus and Cupriavidus necator type SCL production (See e.g., S. Slater, T. Gallaher, and D. Dennis, “Production of poly-(3-hydroxybutyrate-co-3-hydroxyvalerate) in a recombinant Escherichia coli strain.,” Appl. Environ. Microbiol., vol. 58, no. 4, pp. 1089-1094, 1992, doi: 10.1128/AEM.58.4.1089-1094.1992; and J. -I. Choi, S. Y. Lec, and K. Han, “Cloning of the Alcaligenes latus Polyhydroxyalkanoate Biosynthesis Genes and Use of These Genes for Enhanced Production of Poly (3-hydroxybutyrate) in Escherichia coli,” APPL Env. MICROBIOL, vol. 64, p. 7, 1998), Pseudomonas type MCL production (See e.g., Q. Wang, R. C. Tappel, C. Zhu, and C. T. Nomura, “Development of a New Strategy for Production of Medium-Chain-Length Polyhydroxyalkanoates by Recombinant Escherichia coli via Inexpensive Non-Fatty Acid Feedstocks,” Appl. Environ. Microbiol., vol. 78, no. 2, pp. 519-527, January 2012, doi: 10.1128/AEM.07020-11; R. C. Tappel, J. M. Kucharski, J. M. Mastroianni, A. J. Stipanovic, and C. T. Nomura, “Biosynthesis of Poly [(R)-3-hydroxyalkanoate] Copolymers with Controlled Repeating Unit Compositions and Physical Properties,” Biomacromolecules, vol. 13, no. 9, pp. 2964-2972 September 2012, doi: 10.1021/bm301043t; and S. J. Park, J. P. Park, and S. Y. Lec, “Metabolic engineering of Escherichia coli for the production of medium-chain-length polyhydroxyalkanoates rich in specific monomers,” FEMS Microbiol. Lett., vol. 214, no. 2, pp. 217-222, September 2002, doi: 10.1111/j.1574-6968.2002.tb11350.x), and combinations of both to produce SCL-co-MCL PHA (See e.g., R. A. Scheel, “Increased Production of the Value-Added Biopolymers Poly (R-3-Hydroxyalkanoate) and Poly (γ-Glutamic Acid) From Hydrolyzed Paper Recycling Waste Fines,” Front. Bioeng. Biotechnol., vol. 7, p. 10, 2019; and C. Phithakrotchanakoon, V. Champreda, S. Aiba, K. Pootanakit, and S. Tanapongpipat, “Engineered Escherichia coli for Short-Chain-Length Medium-Chain-Length Polyhydroxyalkanoate Copolymer Biosynthesis from Glycerol and Dodecanoate,” Biosci. Biotechnol. Biochem., vol. 77, no. 6, pp. 1262-1268 June 2013, doi: 10.1271/bbb.130073). The combination of pathways usually results in copolymer with MCL composition that is difficult to control. Strategies to enhance control of the MCL portion of the copolymer include use of highly specific thioesterases to siphon only targeted MCL pre-cursors from fatty acid biosynthesis (See e.g., D. E. Agnew, A. K. Stevermer, J. T. Youngquist, and B. F. Pfleger, “Engineering Escherichia coli for production of C12-C14 polyhydroxyalkanoate from glucose,” Metab. Eng., vol. 14, no. 6, pp. 705-713, November 2012, doi: 10.1016/j.ymben.2012.08.003), or to greatly inhibit β-oxidation such that fatty acid feeds are nearly directly converted to their equivalent MCL PHA (See e.g., R. Davis, P. K. Anilkumar, A. Chandrashekar, and T. R. Shamala, “Biosynthesis of polyhydroxyalkanoates co-polymer in E. coli using genes from Pseudomonas and Bacillus,” Antonie Van Leeuwenhoek, vol. 94, no. 2, pp. 207-216, August 2008, doi: 10.1007/s10482-008-9233-3).

The objective of this work is to establish a method for production from recombinant E. coli LSBJ for production of diverse PHB-co-MCL copolymers, with the MCL PHA being directly created from fatty acids with the same carbon number (See e.g., R. A. Scheel, “Increased Production of the Value-Added Biopolymers Poly (R-3-Hydroxyalkanoate) and Poly (γ-Glutamic Acid) From Hydrolyzed Paper Recycling Waste Fines,” Front. Bioeng. Biotechnol., vol. 7, p. 10, 2019; and T. M. Keenan, J. P. Nakas, and S. W. Tanenbaum, “Polyhydroxyalkanoate copolymers from forest biomass,” J. Ind. Microbiol. Biotechnol., vol. 33, no. 7, pp. 616-626, July 2006, doi: 10.1007/s10295-006-0131-2). It has already been shown that the combination of PhaJ4 with PhaC1 in E. coli LSBJ results in a bacterial system which will convert fatty acids to (R)-3-hydroxyacyl intermediates and the equivalent PHA monomer without induction, yielding a system for synthesis of PHAs with controllable repeating unit composition (See e.g., R. C. Tappel, J. M. Kucharski, J. M. Mastroianni, A. J. Stipanovic, and C. T. Nomura, “Biosynthesis of Poly [(R)-3-hydroxyalkanoate] Copolymers with Controlled Repeating Unit Compositions and Physical Properties,” Biomacromolecules, vol. 13, no. 9, pp. 2964-2972, Scp. 2012, doi: 10.1021/bm301043t; R. C. Tappel, Q. Wang, and C. T. Nomura, “Precise control of repeating unit composition in biodegradable poly (3-hydroxyalkanoate) polymers synthesized by Escherichia coli,” J. Biosci. Bioeng., vol. 113, no. 4, pp. 480-486, April 2012, doi: 10.1016/j.jbiosc.2011.12.004; and R. A. Schcel et al., “Optimizing a Fed-Batch High-Density Fermentation Process for Medium Chain-Length Poly (3-Hydroxyalkanoates) in Escherichia coli,” Front. Bioeng. Biotechnol., vol. 9, p. 618259, February 2021, doi: 10.3389/fbioc.2021.618259). The present disclosure advances the previous system by enabling the PHB fraction of these copolymers to be produced from abundant lignocellulosic waste streams, which can greatly reduce costs and improve process sustainability.

PHB fraction of PHB-co-MCL in this work is demonstrated to be obtainable from

hydrolysates of the beer industry waste brewer's spent grain (BSG). BSG is subject to enzymatic hydrolysis to yield hexose and pentose sugars glucose, xylose, arabinose, which are valuable carbon sources for the fermentative production of biopolymers. These sugars are used for cell growth, and also converted to PHB via heterologous expression of C. necator β-ketothiolase (PhaA) and acetoacetyl-CoA reductase (PhaB), which are polymerized by a broad-specificity

Pseudomonas sp. 61-3 type II polyhydroxyalkanoate synthasc (PhaC1) (see FIG. 1). Co-feeding of sodium decanoate with the BSG hydrolysate enables co-production of PHD polymer of same carbon length as the feed by disabling of β-oxidation genes fadB/fadJ and expression of a broad specificity Pseudomonas putida KT2440 (R)-specific enoyl-CoA hydratasc.

FIG. 1 depicts engineering metabolic pathway for the production of PHB-co-MCL PHA copolymers in E. coli LSBJ harboring pBBRSTQKJ4. Referring to FIG. 1 at [1], E. coli is able to naturally uptake hexose and pentose sugars glucose, xylose and arabinose, which are converted through glycolysis and the pentose phosphate pathway to acetyl-CoA. C. necator derived β-ketothiolase (PhaA) and acetoacetyl-CoA reductase (PhaB) convert acetyl-CoA to (R)-3-hydroxybutyryl-CoA intermediates. Referring to FIG. 1 at [2], mutated fadR gene in E. coli LSBJ allows for constitutive (without induction) expression of fad genes responsible for uptake and conversion of fatty acid substrates to enoyl-CoA intermediates (FadL, FadD, FadE). Deletion of genes coding for FadB and FadJ prevents enoyl-CoA intermediates from completing β-oxidation, and therefore preserves the carbon number of the original fatty acid. A Pseudomonas putida KT2440 (R)-specific enoyl-CoA hydratase (PhaJ4) converts enoyl-CoA to the (R)-3-hydroxyacyl-CoA intermediate. Referring to FIG. 1 at [3] a Pseudomonas sp. 61-3 PHA synthase (PhaC1 (STQK)) engineered for broad substrate uptake incorporates the acyl- and butyryl-CoA intermediates into the growing PHA copolymer.

### Methods and Materials

Brewer's Spent Grains: Brewer's spent grain (100% barley-derived) was obtained from a local brewery (Willow Rock Brewing Company, Syracuse, NY). The samples were collected immediately after separation from the wort. The BSG was dried at 60° C. to reduce the moisture content from 80% to below 10% and stored in a refrigerator at 4° C.

Hydrolysis: Raw BSG was combined in 250 mL Erlenmeyer flasks with pH 5 sodium acetate buffer to a final buffer concentration of 50 mM and a final solid loading of 10% (w/v). Flasks were autoclaved for 15 minutes at 121° C. to prevent microbial growth (See e.g., R. Kataria et al.,

“Surfactant-mediated hydrothermal pretreatment of Ryegrass followed by enzymatic saccharification for polyhydroxyalkanoate production,” Ind. Crops Prod., vol. 111, pp. 625-632, January 2018, doi: 10.1016/j.indcrop.2017.11.029; and T. Pinheiro, E. Coelho, A. Romaní, and L. Domingues, “Intensifying ethanol production from brewer's spent grain waste: use of whole slurry at high solid loadings,” New Biotechnol., June 2019, doi: 10.1016/j.nbt.2019.06.005). After cooling, the commercial enzyme cocktails: Cellic®Ctec2 and Cellic®Htec2 (Novozymes North America, Inc., Franklinton, NC, USA) cellulase and hemicellulase blend were added to each flask at a dosage of 15 FPU/g and 20 U/g biomass, respectively. Flasks were placed in a shaking incubator at 50° C. and 200 RPM for 120 hours. After completion of the hydrolysis, the liquid hydrolysate was separated from insoluble solids by centrifugation at 4000 RPM for 15 min. The hydrolysate was adjusted to pH 7.0 using 10M NaOH and then collected by filtration through 0.2 um PTFE sterile filtration units (Stericup). Samples were analyzed for sugar content by H¹-NMR (See e.g., E. F. Alves, S. K. Bose, R. C. Francis, J. L. Colodette, M. lakovlev, and A. Van Heiningen, “Carbohydrate composition of eucalyptus, bagasse and bamboo by a combination of methods,” Carbohydr. Polym., vol. 82, no. 4, pp. 1097-1101 November 2010, doi: 10.1016/j.carbpol.2010.06.038). The sterile-filtered liquid hydrolysate was stored at 4° C. for later use.

### Construction of the pBBR-STQKABJ4 Vector

The pBBRSTQKABJ4 (hereafter referred to as STJ4) vector was constructed by first amplifying the phaJ4 gene from Pseudomonas putida KT2440 (See e.g., S. Sato, H. Kanazawa, and T. Tsuge, “Expression and characterization of (R)-specific enoyl coenzyme A hydratases making a channeling route to polyhydroxyalkanoate biosynthesis in Pseudomonas putida,” Appl. Microbiol. Biotechnol., vol. 90, no. 3, pp. 951-959, May 2011, doi: 10.1007/s00253-011-3150-5; and Q. Wang and C. T. Nomura, “Monitoring differences in gene expression levels and polyhydroxyalkanoate (PHA) production in Pseudomonas putida KT2440 grown on different carbon sources,” J. Biosci. Bioeng., vol. 110, no. 6, pp. 653-659, December 2010, doi: 10.1016/j.jbiosc.2010.08.001 and Q. Wang, C. Zhu, T. J. Yancone, and C. T. Nomura, “The Effect of Co-Substrate Feeding on Polyhydroxyalkanoate (PHA) Homopolymer and Copolymer Production in Recombinant Escherichia coli LS5218,” j bioprocess engng biorefin, vol. 1, no. 1, pp. 86-92, June 2012, doi: 10.1166/jbeb.2012.1003.) using PCR with the following primers: J4.F.KpnI (ATTGGTACCCAACTGACAACCCGGAGAGT) (SEQ ID NO: 1) and J4.R.EcoRV (ATTGATATCCTGGCAGTTTACGCGAGTG) (SEQ ID NO: 2), PCR was performed according to the manufacturer's protocol using PrimeSTAR HS DNA Polymerase (Takara) and an iCycler thermocycler (Bio-Rad), and the resulting 560 base-pair fragment was gel purified and cloned into the pCR-Blunt (Invitrogen) vector using T4 DNA Ligase (New England Biolabs). The phaJ4 gene was then excised by digestion with KpnI (New England Biolabs) and subsequently cloned into the KpnI site of the pBBRSTQKAB plasmid (See e.g., C. T. Nomura et al., “Effective Enhancement of Short-Chain-Length-Medium-Chain-Length Polyhydroxyalkanoate Copolymer Production by Coexpression of Genetically Engineered 3-Ketoacyl-Acyl-Carrier-Protein Synthase III (fabH) and Polyhydroxyalkanoate Synthesis Genes,” Biomacromolecules, vol. 5, no. 4, pp. 1457-1464 July 2004, doi: 10.1021/bm049959v). The correct orientation of the phaJ4 gene was confirmed by BamHI (New England Biolabs) digestion and analysis of the fragment pattern. All work for cloning and plasmid isolation was performed in E. coli JM109.

### Microorganism and Culture Conditions

The recombinant microorganism E. coli LSBJ and plasmids for transformation are described in Table 2. E. coli LSBJ was made chemically competent and transformed with plasmids by heat shock using standard methods (See e.g., J. Sambrook and D. W. Russell, Molecular Cloning: A Laboratory Manual. CSHL Press, 2001). Bacterial lines were maintained as −80° C. freezer stocks in Lennox Broth (LB; 10 g/L tryptone, 5 g/L yeast extract, 5 g/L NaCl) in 30% glycerol.

TABLE 1
Bacterial strain and plasmids

| | | Antibiotic | |
|---|---|---|---|
| Strain/Plasmid | Characteristics | Selectivity | Reference |
| E. coli LSBJ | E. coli LS5218, | None | R. C. Tappel, Q. Wang, and C. T. Nomura, |
| | ΔfadB/J | | “Precise control of repeating unit |
| | | | composition in biodegradable poly(3- |
| | | | hydroxyalkanoate) polymers synthesized by |
| | | | Escherichia coli,” Journal of Bioscience and |
| | | | Bioengineering, vol. 113, no. 4, pp. 480-486, |
| | | | April 2012, doi: |
| | | | 10.1016/j.jbiosc.2011.12.004.(Herein entirely |
| | | | incorporated by reference) |
| pBBRSTQKAB | pBBR1MCS-2, | Kanamycin, | C. T. Nomura et al., “Effective Enhancement |
| | phaC1 (STQK), | 50 mg/L | of Short-Chain-Length-Medium-Chain- |
| | phaA, phaB, | | Length Polyhydroxyalkanoate Copolymer |
| | | | Production by Coexpression of Genetically |
| | | | Engineered 3-Ketoacyl-Acyl-Carrier-Protein |
| | | | Synthase III (f abH) and |
| | | | Polyhydroxyalkanoate Synthesis Genes,” |
| | | | Biomacromolecules, vol. 5, no. 4, pp. 1457- |
| | | | 1464, July 2004, doi: |
| | | | 10.1021/bm049959v.(Herein entirely |
| | | | incorporated by reference) |
| pBBRC1J4SII | pBBR1MCS-2, | Kanamycin, | R. C. Tappel, Q. Wang, and C. T. Nomura, |
| | phaC1 (STQK), | 50 mg/L | “Precise control of repeating unit |
| | phaJ4 | | composition in biodegradable poly(3- |
| | | | hydroxyalkanoate) polymers synthesized by |
| | | | Escherichia coli,” Journal of Bioscience and |
| | | | Bioengineering, vol. 113, no. 4, pp. 480-486, |
| | | | April 2012, doi: 10.1016/j.jbiosc.2011.12.004. |
| | | | (Herein entirely incorporated by reference) |
| pBBRSTQKABJ4 | pBBR1MCS-2, | Kanamycin, | This study |
| | phaC1 (STQK), | 50 mg/L | |
| | phaA, phaB, phaJ4 | | |

The genetically engineered strain E. coli LSBJ (derived from the K-12 strain E. coli LS5218) is notable for its constitutive atoC expression mutated FadR, modifications which make the strain particularly adept at the uptake of fatty acid substrates up to 14 carbons long. LSBJ has undergone deletions of the genes fadB and fadJ, resulting in a bacterium which is unable to the break down fatty acyl-CoA substrates such as those produced by β-oxidation. E. coli LSBJ exhibits the additional advantage of not having the capability to degrade PHAs that is inherent to native producers (See e.g., R. C. Tappel, Q. Wang, and C. T. Nomura, “Precise control of repeating unit composition in biodegradable poly (3-hydroxyalkanoate) polymers synthesized by Escherichia coli,” J. Biosci. Bioeng., vol. 113, no. 4, pp. 480-486, April 2012, doi: 10.1016/j.jbiosc.2011.12.004).

Cultures of recombinant E. coli harboring the plasmid STJ4 were freshly prepared for each experiment by dilution-streaking the freezer stock onto solid media plates of LB, kanamycin (50 μg/L) and agar (15 g/L). The solid plates were incubated at 37° C. for 16, after which individual colonies were selected for preparation of seed culture in 10 mL test tubes containing LB and kanamycin (μg/L). Seed cultures were incubated in a shaking incubator at 37° C. and 200 RPM for 16 hours before inoculation (0.5% v/v) of shake flasks.

Growth was conducted in 500 mL baffled shake flasks containing LB media, 50 μg/L kanamycin, 2 g/L sodium decanoate with additional glucose or BSG hydrolysate added to a total volume of 100 mL. Cells were grown for 48 hours in a shaking incubator set to 30° C. and 200 RPM. Cells were harvested by centrifugation at 3716×g for 15 min, washed successively with 40% ethanol and water, re-suspended in a minimal volume of deionized water, and stored at −80° C. overnight. The frozen cells were then lyophilized for 48 hours to remove any residual moisture in preparation for gravimetric measurement of cell yield and GC analysis for PHA content.

### Analytical Methods

Hydrolysis samples were analyzed for monosaccharide content by H¹-NMR spectroscopy on a 600 MHz Bruker Avance by combining 900 μL of filtered sample with 100 μL of 0.2 M glucosamine internal standard. Identification and quantification of the characteristic monosaccharide and furan data were processed using the TOPSPIN v4.0.9 software from Bruker BioSpin. A detailed discussion of the quantification algorithm has been presented elsewhere (See e.g., E. F. Alves, S. K. Bose, R. C. Francis, J. L. Colodette, M. lakovlev, and A. Van Heiningen, “Carbohydrate composition of eucalyptus, bagasse and bamboo by a combination of methods,” Carbohydr. Polym., vol. 82, no. 4, pp. 1097-1101 November 2010, doi: 10.1016/j.carbpol.2010.06.038; and S. K. Bosc, V. A. Barber, E. F. Alves, D. J. Kiemle, A. J. Stipanovic, and R. C. Francis, “An improved method for the hydrolysis of hardwood carbohydrates to monomers,” Carbohydr. Polym., vol. 78, no. 3, pp. 396-401, October 2009, doi: 10.1016/j.carbpol.2009.04.015). PHA content of the cells was measured by GC-FID using a methanolysis procedure adapted from previously reported methods (See e.g., R. A. Scheel, “Increased Production of the Value-Added Biopolymers Poly (R-3-Hydroxyalkanoate) and Poly (γ-Glutamic Acid) From Hydrolyzed Paper Recycling Waste Fines,” Front. Bioeng. Biotechnol., vol. 7, p. 10, 2019). Approximately 15 mg of lyophilized cell was measured into 15 mL pressure tubes. To each tube, 2 mL of 15% (v/v) H2SO4 in MeOH and 2 mL of chloroform were added and vortexed for 7-8 seconds each. Tubes were then placed into a heating block set to 100° C. for 140 minutes. After heating, tubes were removed from heating block and allowed to cool for 15 min. After cooling, 1 mL of nanopure water and 500 μL of 0.25% (v/v) methyl octanoate in chloroform (internal standard) were added. Each tube was vortexed for 7-8 seconds and then centrifuged at 700 RPM for 5 min. The organic (bottom) layer was removed using a glass Pasteur pipette and filtered through a 0.2 μm PTFE filter set in a vacuum manifold (Millex Samplicity). The filtrate was collected into GC vials and measured for PHA content on a Shimadzu GC-2010 gas chromatograph equipped with a Restek RTX-5 column and flame ionization detector. Samples were run in split mode (40:1) with an injection volume of 1 μL and an injection temperature of 280° C., a detector temperature of 310° C., and a heating profile for the oven as follows: 100° C. for 7 minutes, ramp 8° C./min to 280° C., hold for 2 min, ramp 20° C./min to 310° C., hold for 2 min (See e.g., R. C. Tappel, Q. Wang, and C. T. Nomura, “Precise control of repeating unit composition in biodegradable poly (3-hydroxyalkanoate) polymers synthesized by Escherichia coli,” J. Biosci. Bioeng., vol. 113, no. 4, pp. 480-486, April 2012, doi: 10.1016/j.jbiosc.2011.12.004). Chromatographic data were analyzed using Shimadzu software.

PHA samples were prepared for NMR analysis by chloroform extraction and methanol purification. Approximately 100 mg of lyophilized cells were weighed into 15 mL pressure tubes. 6 mL of chloroform was added to each tube. Tubes were then vortexed for 7-8 seconds, followed by heating at 100° C. in a heating block for 1 hour. After allowing the chloroform to separate from the cell debris by settling overnight, the extracted PHA was in the chloroform layer was dispensed using glass Pasteur pipettes into 50 mL centrifuge tubes containing 40 mL of ice-cold methanol. The tubes were centrifuged at 8500 RPM and 4° C. for 20 min, followed by pouring off the impurity-containing supernatant. Residual methanol was allowed to evaporate from purified PHA samples overnight. Purified PHA was then dissolved in deuterated chloroform at 15 g/L and subject to H¹-NMR and C¹³-NMR analysis on a Bruker 600 MHZ NMR.

### Results

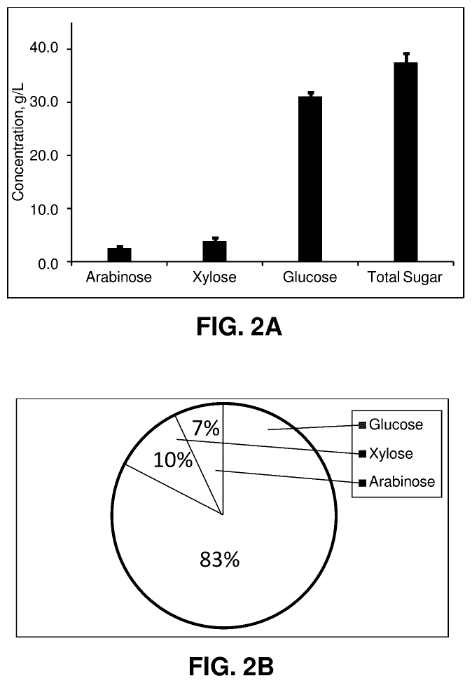

BSG was hydrolyzed to release the fermentable sugars glucose, xylose, and arabinose. Sec e.g., FIGS. 2A and 2B the depict the concentration of glucose, xylose, and arabinose sugars in the BSG hydrolysate. BSG is often subject to a pretreatment to increase hydrolysis and process yields (See e.g., T. Pinheiro, E. Coelho, A. Romaní, and L. Domingues, “Intensifying ethanol production from brewer's spent grain waste: use of whole slurry at high solid loadings,” New Biotechnol., June 2019, doi: 10.1016/j.nbt.2019.06.005; and J. A. Rojas-Chamorro, I. Romero, J. C. López-Linares, and E. Castro, “Brewer's spent grain as a source of renewable fuel through optimized dilute acid pretreatment,” Renew. Energy, vol. 148, pp. 81-90, April 2020, doi: 10.1016/j.renenc.2019.12.030), but previous work demonstrated that the overall process yield was actually decreased by inclusion of a pretreatment process, which was likely an effect of inhibitor release which depressed cell PHA productivity. The resulting hydrolysate herein contained 37 g/L of total sugars, which agrees with our previous work and is concentrated enough for use in fermentation experiments.

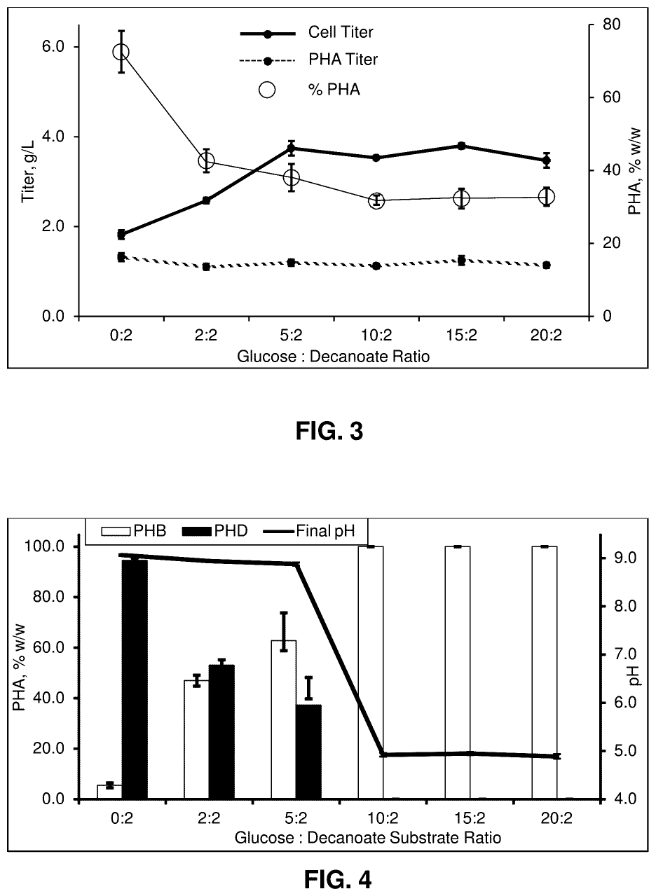

Co-production of PHB and PHD homopolymer in E. coli LSBJ harboring STJ4 was investigated with varying amounts of glucose and a constant amount of decanoate. It was found that as the concentration of glucose increased, the % inclusion of total PHA decreased logarithmically to a constant value of about 32%. Due to an increase in cell titer from higher abundance of carbon sources for cell growth, the total PHA titer remained relatively constant irrespective of glucose concentration (See e.g., FIG. 3) FIG. 3 depicts a cell titer and PHA titer (lines) and % PHA inclusion (markers) after 48 h of E. coli LSBJ STJ4 shake flask growth with varying ratios of glucose to decanoate starting substrate. The ratio of PHB: PHD increased with initial glucose concentration up to 5 g/L. However, it was observed that the production of PHD ceased once starting glucose concentrations were greater than or equal to 10 g/L, which coincided with a change in final pH from basic to acidic conditions.

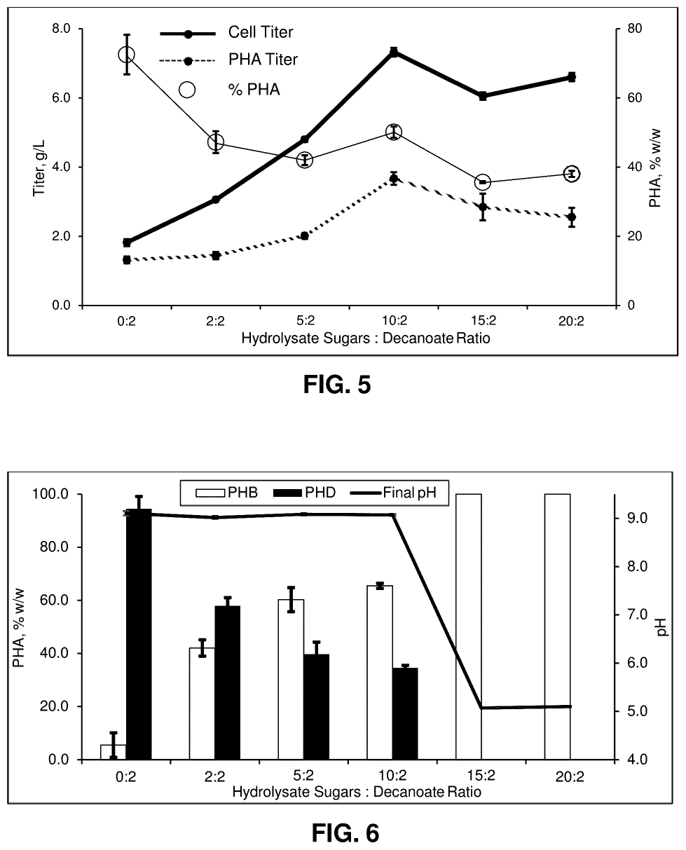

The introduction of BSG hydrolysate as carbon source presented a similarly negative relationship with initial sugar concentration and % PHA inclusion. However, total PHA titer nonetheless increased with initial sugar concentration due to the strongly positive response of cell titer to increasing BSG hydrolysate dosage. A maximum PHA and cell titer of 3.7 g/L and 7.3 g/L, respectively, was observed at 10 g/L initial BSG-derived total sugar (FIG. 5). FIG. 5 refers to cell titer and PHA titer (lines) and % PHA inclusion (markers) after 48 h of E. coli LSBJ STJ4 shake flask growth with varying ratios of hydrolysate sugars to decanoate starting substrate.

The ratio of PHB: PHD increased with additional hydrolysate sugars dosed up to 10 g/L. As with the glucose, the cessation of PHB and PHD co-production was synchronous with the change in final culture pH from basic to acidic conditions (See e.g., FIG. 6). FIG. 6 depicts PHB-co-PHD copolymer ratios (bars) and final pH (line) after 48 h of E. coli LSBJ STJ4 shake flask growth with varying ratios of hydrolysate sugars to decanoate starting substrate.

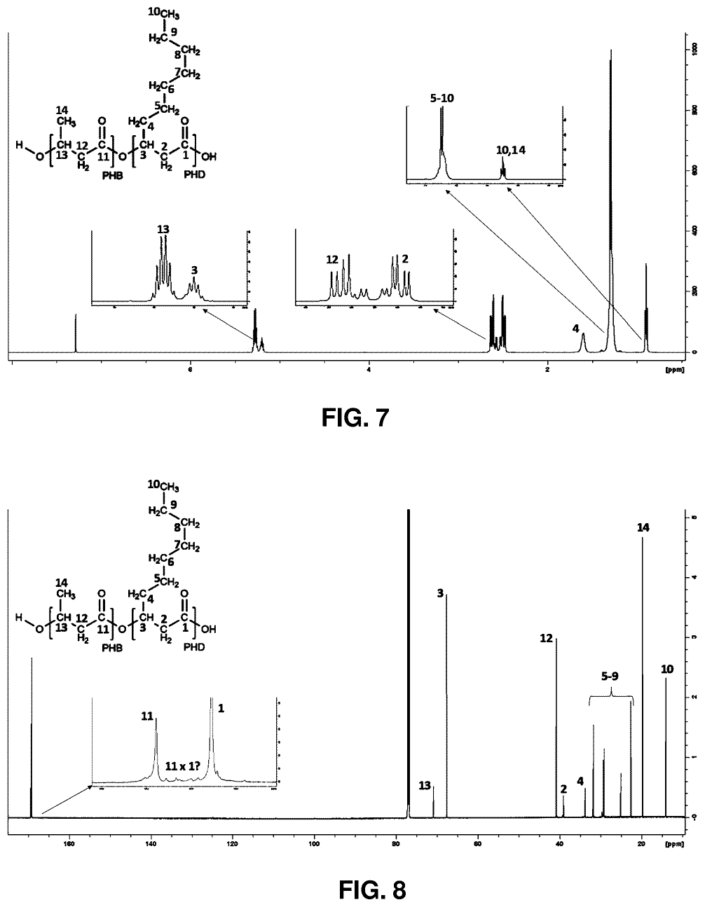

Referring now to FIG. 7, FIG. 7 depicts H¹-NMR data of the purified polymer confirmed production of PHB and PHD monomer units. More specifically, FIG. 7 depicts H¹-NMR spectra of purified polymer produced by engineered E. coli LSBJ. The ratio of integrated intensity of the methine peaks for this sample (13 and 3) indicates a mass ratio of 53% PHB and 47% PHD, which matches well with the GC data (53% PHB and 47% PHD).

C¹³-NMR data is an often-used method to investigate production of copolymer PHAs (See e.g., M. Li et al., “Engineering Pseudomonas entomophila for synthesis of copolymers with defined fractions of 3-hydroxybutyrate and medium-chain-length 3-hydroxyalkanoates,” Metab. Eng., vol. 52, pp. 253-262, March 2019, doi: 10.1016/j.ymben.2018.12.007; and L. Shang, S. C. Yim, H. G. Park, and H. N. Chang, “Sequential Feeding of Glucose and Valerate in a Fed-Batch Culture of Ralstonia eutropha for Production of Poly (hydroxybutyrate-co-hydroxyvalerate) with High 3-Hydroxyvalerate Fraction,” Biotechnol. Prog., vol. 20, no. 1, pp. 140-144, September 2004, doi: 10.1021/bp0342320). At about 169 ppm the diad-dependent carbonyl peaks for each monomer fraction can be observed, with covalently linked monomer units causing diad interactions which result in unique chemical environments and additional carbonyl peaks.

The C¹³-NMR spectra of our sample confirms the presence of the distinct PHB and PHD polymers, with some small amount of non-homopolymer diad interaction (FIG. 8) FIG. 8 depicts C¹³-NMR spectra of purified polymer produced by engineered E. coli LSBJ.

### Discussion

Hydrolysis of the untreated BSG yielded a sugar-rich liquid hydrolysate, with a major glucose fraction (83%) and a minor mixed pentose fraction (17%) (FIGS. 2A and 2B). Cereal grain residues, such as that derived from corn (dried distillers grains with solubles, DDGs) or barley (BSG), are an abundant low-cost feedstock rich in hexose and pentose sugars that can be used for a number of biotechnological upcycling applications (See e.g., S. I. Mussatto, “Brewer's spent grain: a valuable feedstock for industrial applications: Brewer's spent grain and its potential applications,” J. Sci. Food Agric., vol. 94, no. 7, pp. 1264-1275 May 2014, doi: 10.1002/jsfa.6486; and A. Chatzifragkou et al., “Biorefinery strategies for upgrading Distillers' Dried Grains with Solubles (DDGS),” Process Biochem., vol. 50, no. 12, pp. 2194-2207 December 2015, doi: 10.1016/j.procbio.2015.09.005).

E. coli is an advantageous organism to use for production of heterologous biomaterials based on lignocellulosic feedstocks because of its natural ability to utilize not only a wide range of hexose and pentose sugars, but also acetate, lactate, and even uronic acids (See e.g., H. Lawford and J. Rousseau, “Fermentation of Biomass-Derived Glucuronic Acid by pet Expressing Recombinants of E. coli B,” Appl. Biochem. Biotechnol., vol. 63, p. 21, 1997; and H. G. Lawford and J. Rousseau, “Mannose Fermentation by an Ethanologenic Recombinant Escherichia cdl,” Biotechnol. Lett., vol. 15, no. 6, pp. 615-620, June 1993). However, this advantage in complete lignocellulosic hydrolysate nutrient utilization is tempered by the multi-tiered hierarchal metabolism of sugars by E. coli, in a phenomenon known as carbon-catabolite repression (CCR). In this case, CCR is observed as E. coli first consuming glucose to exhaustion, then arabinose, and finally xylose, which is a less productive fermentation than if the culture had access to an equivalent quantity of only glucose (See e.g., T. A. Desai and C. V. Rao, “Regulation of Arabinose and Xylose Metabolism in Escherichia coli,” Appl. Environ. Microbiol., vol. 76, no. 5, pp. 1524-1532 March 2010, doi: 10.1128/AEM.01970-09). Beyond the substrate advantages of E. coli generally, the strain E. coli LSBJ in particular is effective at overcoming the inhibitory effects of the common lignocellulosic hydrolysate component acetate. The BSG hydrolysate here was measured to contain 5.6 g/L of acetate, which above even low concentrations (>0.5 g/L) in fermentation media can significantly reduce the growth rate of E. coli strains on sugars (See e.g., Eiteman, M. A., & Altman, E. (2006). Overcoming acetate in Escherichia coli recombinant protein fermentations. Trends in Biotechnology, 24 (11), 530-536. doi.org/10.1016/j.tibtech.2006.09.001; Nakano, K., M. Rischke, S. Sato, and H. Märkl. “Influence of acetic acid on the growth of Escherichia coli K12 during high-cell-density cultivation in a dialysis reactor.” Applied microbiology and biotechnology 48, no. 5 (1997): 597-601.). However, by way of an atoC mutation allowing for constitutive expression of pathways for acetate utilization, E. coli LSBJ is actually well-suited to grow on mediums rich in acetate (See e.g., S. Slater, T. Gallaher, and D. Dennis, “Production of poly-(3-hydroxybutyrate-co-3-hydroxyvalerate) in a recombinant Escherichia coli strain.,” Appl. Environ. Microbiol., vol. 58, no. 4, pp. 1089-1094, 1992, doi: 10.1128/AEM.58.4.1089-1094.1992) The combination of wide-substrate uptake and enhanced resistance to and utilization of acetate make E. coli LSBJ a primary candidate for fermentation of lignocellulosic hydrolysates that contain a mixture of hexose sugars, pentose sugars, and acetate.

The one-pot targeted production of PHB and PHD polymers by E. coli using glucose and decanoate is reported here for the first time. Many previous works have demonstrated similar processes but were limited by an incomplete disruption of the fatty acid β-oxidation pathway, which results in step-wise reduction in carbon number of the single fatty acid feed and consequently production of a mixture of MCL PHAs (See e.g., C. Phithakrotchanakoon, V. Champreda, S. Aiba, K. Pootanakit, and S. Tanapongpipat, “Engineered Escherichia coli for Short-Chain-Length Medium-Chain-Length Polyhydroxyalkanoate Copolymer Biosynthesis from Glycerol and Dodecanoate,” Biosci. Biotechnol. Biochem., vol. 77, no. 6, pp. 1262-1268 June 2013, doi: 10.1271/bbb.130073; R. Davis, P. K. Anilkumar, A. Chandrashekar, and T. R. Shamala, “Biosynthesis of polyhydroxyalkanoates co-polymer in E. coli using genes from Pseudomonas and Bacillus,” Antonie Van Leeuwenhoek, vol. 94, no. 2, pp. 207-216, August 2008, doi: 10.1007/s10482-008-9233-3; and T. Pinheiro, E. Coelho, A. Romaní, and L. Domingues, “Intensifying ethanol production from brewer's spent grain waste: use of whole slurry at high solid loadings,” New Biotechnol., June 2019, doi: 10.1016/j.nbt.2019.06.005). The key advance herein is the complete disruption of the β-oxidation pathway by deletion of genes in E. coli coding for both aerobic (fadB) and anaerobic (fadJ) (S)-specific enoyl-CoA hydratases, which in combination with expression of a broad-specificity (R)-specific enoyl-CoA hydratase and type II polyhydroxyalkanoate synthase allows for the production of MCL PHAs with the same carbon number as the starting fatty acid feed (See e.g., R. C. Tappel, Q. Wang, and C. T. Nomura, “Precise control of repeating unit composition in biodegradable poly (3-hydroxyalkanoate) polymers synthesized by Escherichia coli,” J. Biosci. Bioeng., vol. 113, no. 4, pp. 480-486, April 2012, doi: 10.1016/j.jbiosc.2011.12.004). This advancement allows for a higher level of control in PHB-MCL PHA production, which is desirable in consideration of scalable bioprocesses to convert cheap resources into biopolymers with properties determinable from feed composition.

As initially available concentrations of glucose increased in the shake flasks was increased from 0 g/L to 5 g/L the total PHA cell inclusion decreased and the cell titer increased, with further increases in initial glucose to 20 g/L resulting in no further changes in PHA inclusion or cell titer (See FIG. 3). This coincided with a unit-step drop in final culture acidity from a pH of 9 to a pH of 5 (See FIG. 4). FIG. 4 depicts PHB-co-PHD copolymer ratios (bars) and final pH (line) after 48 h of E. coli LSBJ STJ4 shake flask growth with varying ratios of glucose to decanoate starting substrate. What was most likely incurred by the additional glucose was metabolic or acetate overflow, wherein excessive amounts of glucose in the media cause E. coli cells to excrete acetate into the media. Excessive acetate in the media will lower the cell cytoplasm pH, inhibiting ATP synthesis and overall growth (See e.g., V. Bernal, S. Castaño-Cerezo, and M. Cánovas, “Acetate metabolism regulation in Escherichia coli: carbon overflow, pathogenicity, and beyond,” Appl. Microbiol. Biotechnol., vol. 100, no. 21, pp. 8985-9001 November 2016, doi: 10.1007/s00253-016-7832-x). However, what is interesting to note here is the drop in pH coinciding with a complete cessation of PHA production and cell growth, which would normally continue with E. coli LSBJ even in acetate-rich acidic media. However, with acetate overflow the decanoate feed becomes protonated and much more toxic. The combination of low-pH and lipid-soluble weak acids is actually a common food preservation technique to inhibit microbial growth, and the toxicity of short-chain fatty acids on E.coli has been well-documented (See e.g., J. B. Russell, “Another explanation for the toxicity of fermentation acids at low pH: anion accumulation versus uncoupling,” J. Appl. Bacteriol., vol. 73, no. 5, pp. 363-370, November 1992, doi: 10.1111/j.1365-2672.1992.tb04990.x). So in this fermentation, the shift to acetate overflow activated the toxicity of the decanoate fatty acid and effectively stopped the fermentation.

A similar relationship was observed with increasing equivalent concentrations of hydrolysate derived sugars. After surpassing a threshold initial sugar concentration, the media conditions became acidic due to acetate overflow, greatly increasing the toxicity of the fatty acid feed and effectively terminating the fermentation early (See FIGS. 5 and 6). The apparent threshold sugar concentration to induce acetate overflow metabolism is higher when dosing BSG hydrolysate-derived mixed hexose and pentose sugars. This can be explained by the sugar profile of the feed (See FIG. 3) and the sequential sugar metabolism caused by CCR in E. coli, meaning that at 10 g/L hydrolysate sugars the cells were actually exposed to 8.3 g/L initial glucose, and then 0.7 g/L arabinose, and finally 1 g/L xylose. Given the tested concentrations in the glucose-only experiments were 5 and 10 g/L, it is likely the actual threshold concentration to induce acetate-overflow is closer to 8.3 g/L glucose as seen with the hydrolysate-based media.

Future experiments would be well-designed to consider this observed toxicity effect in co-feeding of sugars and fatty acid for PHA production. Strategies to overcome this include fermentation with sugars and fatty acid supplied in fed-batch mode. Operation in fed-batch mode is commonly used to avoid the growth-inhibitory effects of toxic feeds, keeping E. coli cells in exponential phase of growth and enhancing PHA accumulation (See e.g., F. I. Mohd Fadzil, S. Mizuno, A. Hiroc, C. T. Nomura, and T. Tsuge, “Low Carbon Concentration Feeding Improves Medium-Chain-Length Polyhydroxyalkanoate Production in Escherichia coli Strains With Defective β-Oxidation,” Front. Bioeng. Biotechnol., vol. 6, p. 178, November 2018, doi: 10.3389/fbioc.2018.00178). Alternatively, metabolic engineering of E. coli by deletion of genes coding for phosphotransferase (ptaG) would allow simultaneous uptake of sugars and fatty acid and therefore decrease the concentration of potential toxins before they can be activated by a pH shift (See e.g., R. Li, Q. Chen, P. G. Wang, and Q. Qi, “A novel-designed Escherichia coli for the production of various polyhydroxyalkanoates from inexpensive substrate mixture,” Appl. Microbiol. Biotechnol., vol. 75, no. 5, pp. 1103-1109 July 2007, doi: 10.1007/s00253-007-0903-2).

The use of BSG hydrolysate as a carbon source for fermentation resulted in higher cell titers and PHA titers as compared to pure sugars, which has been demonstrated before. BSG is rich in not only the fibers valued for hydrolysis to sugars, but also proteins, vitamins and minerals (See e.g., A. Chetrariu and A. Dabija, “Brewer's Spent Grains: Possibilities of Valorization, a Review,” Appl. Sci., vol. 10, no. 16, p. 5619 August 2020, doi: 10.3390/app10165619). It is possible that during enzymatic hydrolysis many of these compounds are extracted into the hydrolysate and therefore used by the E. coli during fermentation to enhance their growth. The other obvious major carbon source is additional acetate supplied in BSG hydrolysate. Acetate is used by the E. coli LSBJ to support cell growth, cell energy production, and production of PHB.

H¹-NMR spectroscopy was used to confirm the presence of comonomers PHB and PHD in the extracted polymer sample (See FIG. 7) using correlations established in previous works (See e.g., X. Gao et al., “Production of copolyesters of 3-hydroxybutyrate and medium-chain-length 3-hydroxyalkanoates by E. coli containing an optimized PHA synthase gene,” Microb. Cell Factories, vol. 11, no. 1, p. 130, 2012, doi: 10.1186/1475-2859-11-130). C¹³-NMR spectroscopy was further used to investigate the structural relation of the produced PHB and PHD (See FIG. 8). Comonomers of PHA can be produced in biological systems with any of a variety of chemical compositional distributions (CCD), such as distinct homopolymers, random copolymers, block copolymers, and combinations of the above. Fermentation conditions, such as staged feeding of specific precursors, or biological predispositions, such as the co-expression of PHA synthases with distinct and separate substrate specificities, can influence the CCD. The carbonyl peak of PHA monomers is sensitive to its unique chemical environment and can be used to qualify the presence of individual PHA monomers and covalently linked copolymers. A Bernoullian statistical model can be used to indicate the degree of copolymerization in a population of comonomers by calculation of a parameter D based on relative intensity of the diad-dependent carbonyl resonances (See e.g., N. Kamiya, Y. Yamamoto, Y. Inoue, R. Chujo, and Y. Doi, “Microstructure of bacterially synthesized poly (3-hydroxybutyrate-co-3-hydroxyvalerate),” Macromolecules, vol. 22, no. 4, pp. 1676-1682 April 1989, doi: 10.1021/ma00194a030). D value of 1 indicate a statistically random copolymer, with values less than 1 indicating alternating copolymer, and values greater than 1 indicating blocky copolymer. The D value was calculated here to be 48, which is indicative of very blocky copolymer, or mostly homopolymer and a small amount of random copolymer.

Given the above analysis of polymer sample, and the knowledge that E. coli LSBJ will sequentially uptake sugars before fatty acids due to CCR, and the fact that in PHA biosynthesis nutrient uptake is much faster than the rate-limiting step of polymerization (See e.g., N. V. Mantzaris, A. S. Kelley, P. Daoutidis, and F. Srienc, “A population balance model describing the dynamics of molecular weight distributions and the structure of PHA copolymer chains,” Chem. Eng. Sci., vol. 57, no. 21, pp. 4643-4663 November 2002, doi: 10.1016/S0009-2509(02)00370-6; and E. N. Pederson, C. W. J. McChalicher, and F. Sricnc, “Bacterial Synthesis of PHA Block Copolymers,” Biomacromolecules, vol. 7, no. 6, pp. 1904-1911 June 2006, doi: 10.1021/bm0510101), the following model can be constructed for E. coli LSBJ STJ4 co-fed sugar and fatty acid in batch mode:

Glucose, arabinose, and xylose are metabolized, in that order, providing cellular energy, cell biomass, and PHB. During this time, PHB synthesis is randomly initiated, propagated, and terminated.

As sugars are depleted, cell growth slows and the fad genes are re-activated for fatty acid uptake, which is converted to PHD. As nutrient uptake is significantly faster than PHB synthesis and chain termination, there exists a viable population of propagating PHB chains to which the newly incoming PHD monomer can attach to, forming blocky PHB-co-PHD copolymer.

The rate of PHB-co-PHD chain termination is much higher than the rate of decanoate assimilation and PHD formation, resulting in much of the remaining decanoate being exhausted to form PHD homopolymer.

The resulting PHA from this process is therefore a mixture of PHB homopolymer, PHD homopolymer, and PHB-co-PHD block copolymer of the A-B block type. The inclusion of even a small amount of block copolymer can have pronounced effects on the material properties of PHA blends by acting as a compatibilizer between separate phases, reducing interfacial tension, improving miscibility, and overall creating a more homogenous material (See e.g, R. D. Ashby, D. K. Y. Solaiman, and T. A. Foglia, “Synthesis of Short-/Medium-Chain-Length Poly (hydroxyalkanoate) Blends by Mixed Culture Fermentation of Glycerol,” Biomacromolecules, vol. 6, no. 4, pp. 2106-2112 July 2005, doi: 10.1021/bm058005h). The sequential pulsed-feeding supply of PHB and PHD precursors in a fed-batch fermentation could increase the amount of block copolymer, as well as the number of blocks within the polymer (See e.g., E. N. Pederson, C. W. J. McChalicher, and F. Srienc, “Bacterial Synthesis of PHA Block Copolymers,” Biomacromolecules, vol. 7, no. 6, pp. 1904-1911 June 2006, doi: 10.1021/bm0510101).

Given the robust nature of E. coli LSBJ STJ4, the biotechnological system described herein could be extended as a production scheme to create a great number of PHB-co-MCL PHA types. The source of lignocellulosic hydrolysate could be changed to any of a number of abundant waste feedstocks, such as rice or wheat bran, and the fatty acid feed could be changed to create any PHA from 4-14 carbons in length, including functionalized or otherwise unusual MCL PHA precursors (See e.g., R. A. Scheel et al., “Optimizing a Fed-Batch High-Density Fermentation Process for Medium Chain-Length Poly (3-Hydroxyalkanoates) in Escherichia coli,” Front. Bioeng. Biotechnol., vol. 9, p. 618259, February 2021, doi: 10.3389/fbioc.2021.618259). The result here is a great advance toward the creation of a platform for sustainable production for versatile and highly functional PHA copolymers. Future work would be well-aimed to investigate the claims of interchangeability of lignocellulosic hydrolysate and fatty acid feed. Importantly, the effect of different fed-batch fermentation schemes should also be investigated for the possibility of improving PHA titers and altering copolymer composition.

### Conclusion

This work demonstrates for the first time the one-pot production of PHB, targeted fatty acid MCL PHA, and PHB-co-MCL block copolymer in E. coli by co-feeding of BSG enzymatic hydrolysate and dedicated sodium decanoate feed. This was accomplished by construction and expression of the plasmid pBBRSTQKABJ4 in β-oxidation fadB/fadJ disabled E. coli LSBJ, which was used to ferment BSG enzymatic hydrolysate and sodium decanoate in shake flasks. Batch mode fermentation of BSG hydrolysate and sodium decanoate yielded a mixture of PHB homopolymer, PHD homopolymer, and PHB-co-PHD block copolymer at maximum PHA titers of 3.7 g/L, with the PHB/PHD ratio tunable according to substrate addition. Higher titers of PHA were unable to be achieved with increasing carbon supply due to acetate overflow manifesting toxic culture conditions.

### Example 2

### Methods

### Gene Deletions

The nonpolar deletion of the ptsG gene was accomplished using the λ red recombinase protocol, as previously described (Datsenko and Wanner, 2000; Jensen et al., 2015). A knockout cassette was generated using PCR with gene-specific primers to amplify the chloramphenicol resistance marker from pKD3 (Table 3). PCR was performed using Phusion Plus DNA polymerase (ThermoFisher Scientific) following the manufacturers recommended protocol for touchdown PCR. The cassette was digested with DpnI to remove the DNA template, analyzed and purified using agarose gel electrophoresis, and concentrated by ethanol precipitation using standard procedures (Sambrook and Russell, 2001). The λ red recombinase was expressed in E. coli LSBJ using plasmid pSIJ8 with 20 mM L-arabinose, and the knockout cassette introduced to cells in mid-exponential phase (OD₆₀₀=0.6) by electroporation (1500V, 5 ms; BTX ECM 399). Successful recombination was determined by antibiotic selection and loci screening using check primers (Table 1). The knockout cassette was removed by the expression of FLP recombinase from the pSIJ8 plasmid with 50 mM L-rhamnose, and successful deletions were confirmed by loss of chloramphenicol resistance and by PCR using loci check primers. The temperature sensitive pSIJ8 plasmid was removed by growth for 12 h at 42° C., and screened for the loss of ampicillin resistance. The new mutant strain LSBJ ΔptsG was named LSBJP (Table 2).

There are a number of ways to reduce gene or gene product activity, e.g., ptsG phosphotransferase in bacteria. These include inhibitors (competitive, non-competitive, uncompetitive), gene deletion, gene mutation, gene silencing, interference with promoters, silencing or interfering RNA (e.g., siRNA, miRNA), and other known techniques. A complete list of strains, plasmids, and primers is shown in Table 2.

TABLE 2
Strains, plasmids, and primers.

| | | Source or |
|---|---|---|
| | Relevant characteristics | reference |
| Escherichia | | |
| coli | | |
| LSBJ | ΔfadB, ΔfadJ, atoC512 | Tappel et al., |
| | (Const), fadR601 | 2012 |
| LSBJP | ΔptsG LSBJ | This study |
| Plasmids | | |
| pSIJ8 | Dual 2 Red recombinase and FLP | Jensen et al. |
| | recombinase expression plasmid; | 2015 |
| | expresses exo, β, and γ genes | |
| | from λ phage under P^{araB} promoter; | |
| | expresses FLP gene under P^{rhaB} | |
| | promoter; contains araC, rhaS, | |
| | and rhaR regulatory elements; | |
| | Amp^{R}; temperature sensitive | |
| | replicon | |
| pKD3 | Chloramphenicol | Datsenko |
| | acetyltransferase flanked | and Wanner, |
| | by FLP recombinase | 2000 |
| | recognition targets, Ap^{R}, Cm^{R} | |
| pBBR-STJ4 | pBBR1MCS-2 derivative | Tappel et al. |
| | containing phaAB, | 2012 |
| | phaJ4, phaC1 | |
| | (STQK) | |

| Primersª | Sequence (5′ to 3′)^{b} |
|---|---|
| pKD3.F.ptsG | ACGTAAAAAAAGCACCCATACTCAGGAGCACTCTCAATTGTG |
| | TAGGCTGGAGCTGCTTC (SEQ ID NO: 11) |
| pK13.R.ptsG | AGCCATCTGGCTGCCTTAGTCTCCCCAACGTCTTACGGAATG |
| | GGAATTAGCCATGGTCC (SEQ ID NO: 12) |
| ptsG.F.check | CCTGTACACGGCGAGGCTCT (SEQ ID NO: 13) |
| ptsG.R.check | AATAACACCTGTAAAAAAGGCAGCC (SEQ ID NO: 14) |

^{a}Forward and reverse primers are denoted with an F or R, respectively.
^{b}Underlined sequences are homologous to the gene to be deleted.

The effect of variable fatty acid and sugar feeding concentrations on relative comonomer inclusion in the newly created strain E. coli ΔptsG STJ4 was investigated. The strain is derivative of E. coli LSBJ, with the additional modification in deleting genes for phosphotransferase ptsG. Phosphotransferase deletion has been shown to allow simultaneous fatty acid and glucose uptake in E. coli, which has been shown to enable simultaneous uptake of hexose and pentose sugars, as well as glucose and fatty acids in other strains of E. coli. Given the nature of the PHA production system in this invention, the ptsG deletion could therefore enable better control and production of PHAs based on mixed feeding strategies of hexose sugars, pentose sugars, and fatty acid precursors.

Cultures of recombinant E. coli LSBJ AptsG harboring the plasmid pBBRSTQKABJ4 were freshly prepared for each experiment by dilution-streaking the freezer stock onto solid media plates of LB, kanamycin (50 μg/L) and agar (15 g/L). The solid plates were incubated at 37° C. for 16 hours, after which individual colonies were selected for seed culture preparation in 10 mL test tubes containing LB and kanamycin (μg/L). Seed cultures were incubated at 37° C. and 200 RPM for 16 hours before inoculation (0.5% v/v).

Fermentation was carried out at a total volume of 100 mL in 500 mL baffled shake flasks at containing: LB media, 50 μg/L kanamycin, and 5 g/L glucose added at hour 7 of growth. Cultures were grown for 48 hours at 30° C. and 200 RPM in a shaking incubator. Harvest of cells was conducted by centrifugation at 3716×g for 15 min. The pellets were washed successively with 70% ethanol, nanopure water, resuspended in a minimal volume of nanopure water, then stored at −80° C. for 2 hours to prepare for lyophilization. The cells were lyophilized for 48 hours to remove residual water. Cell yield was then determined gravimetrically, with PHA content determined by GC.

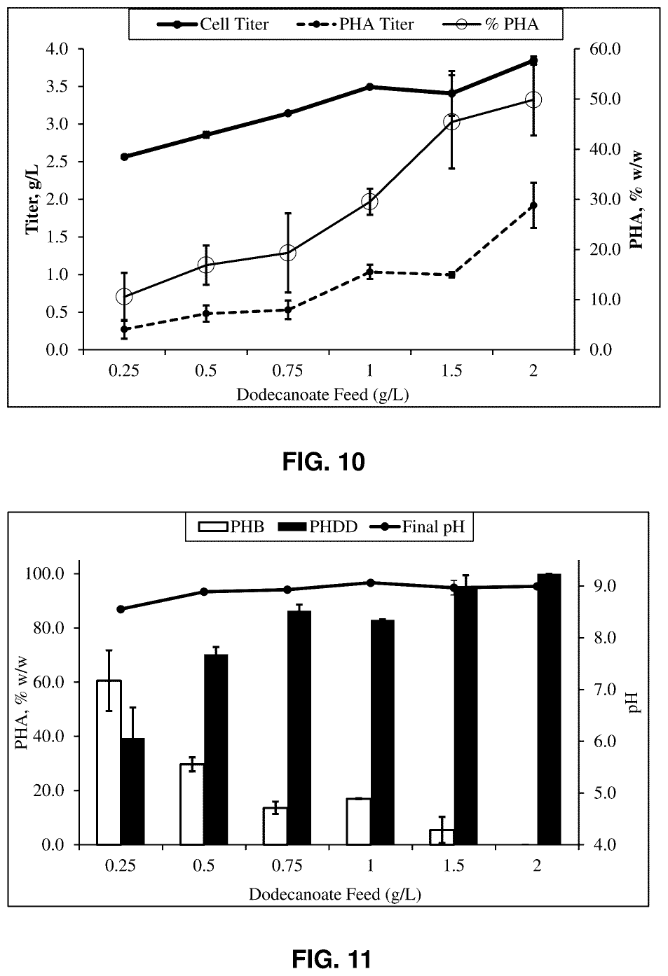

FIG. 10 shows cell titer and PHA titer (lines) and % PHA inclusion (markers) after 48 h of E. coli ptsG STJ4 shake flask growth with varying concentrations of sodium dodecanoate starting substrate.

FIG. 11 shows PHB-co-PHDD comonomer ratios (bars) and final pH (line) after 48 h of E. coliptsG STJ4 shake flask growth with varying concentrations of sodium dodecanoate starting substrate.

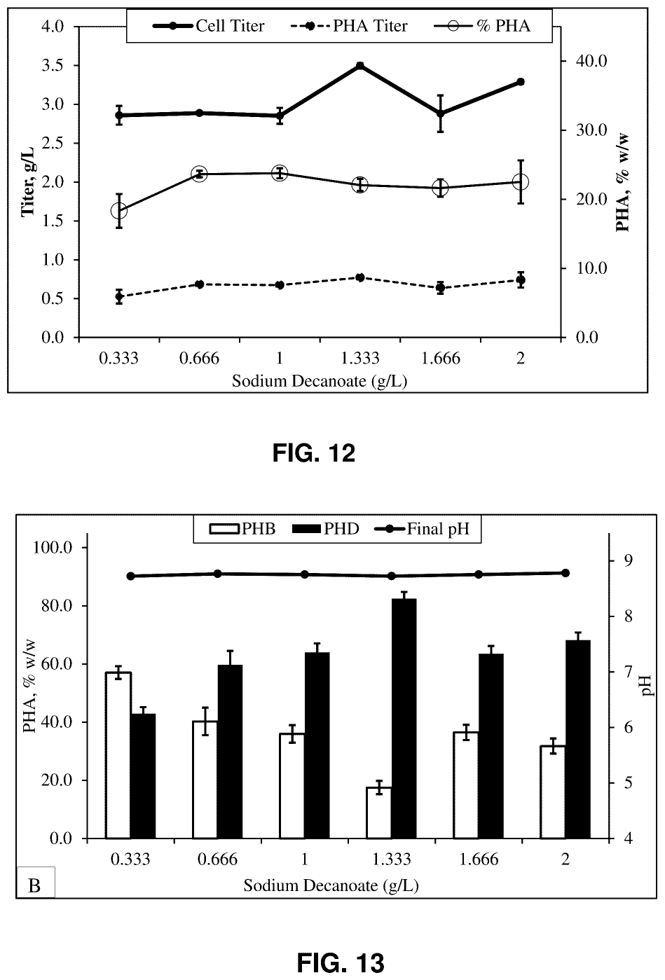

FIG. 12 shows cell titer and PHA titer (lines) and % PHA inclusion (markers) after 48 h of E. coli ptsG STJ4 shake flask growth with varying concentrations of sodium decanoate starting substrate.

FIG. 13 shows PHB-co-PHD comonomer ratios (bars) and final pH (line) after 48 h of E. coliptsG STJ4 shake flask growth with varying concentrations of sodium decanoate starting substrate.

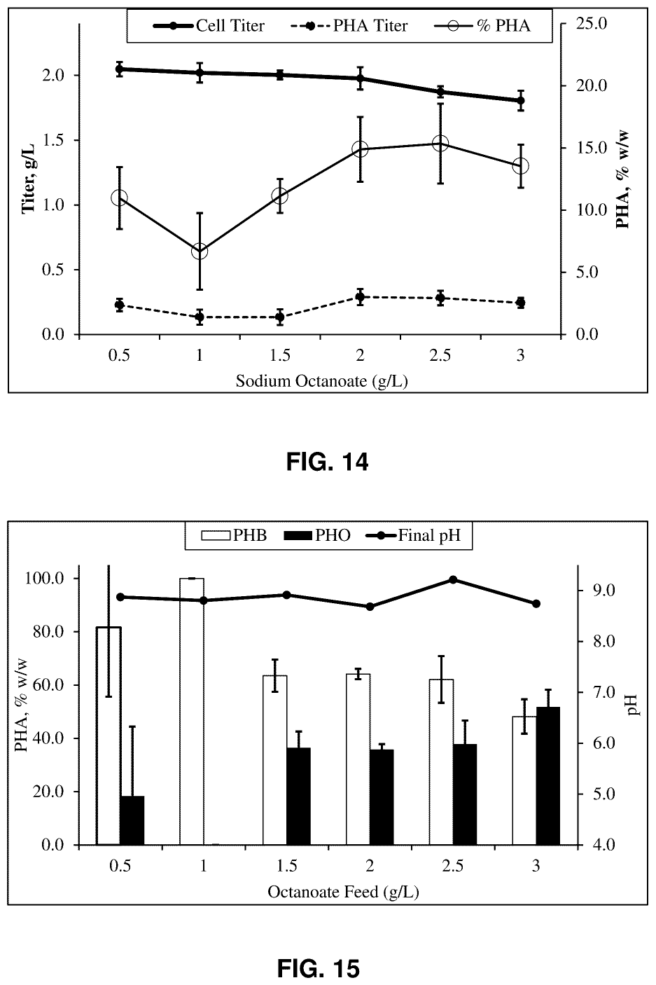

FIG. 14 shows cell titer and PHA titer (lines) and % PHA inclusion (markers) after 48 h of E. coli ptsG STJ4 shake flask growth with varying concentrations of sodium octanoate starting substrate, plus Brij-35.

FIG. 15 shows PHB-co-PHO comonomer ratios (bars) and final pH (line) after 48 h of E. coliptsG STJ4 shake flask growth with varying concentrations of sodium octanoate starting substrate, plus Brij-35.

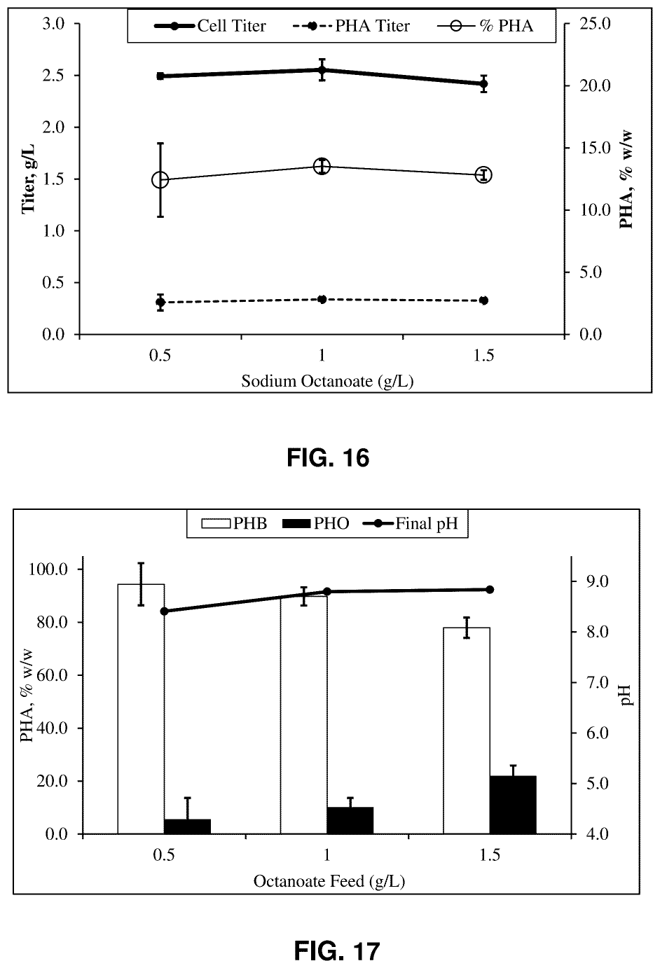

FIG. 16 shows cell titer and PHA titer (lines) and % PHA inclusion (markers) after 48 h of E. coli ptsG STJ4 shake flask growth with varying concentrations of sodium octanoate starting substrate.

FIG. 17 shows PHB-co-PHO comonomer ratios (bars) and final pH (line) after 48 h of E. coli ptsG STJ4 shake flask growth with varying concentrations of sodium octanoate starting substrate, plus Brij-35.

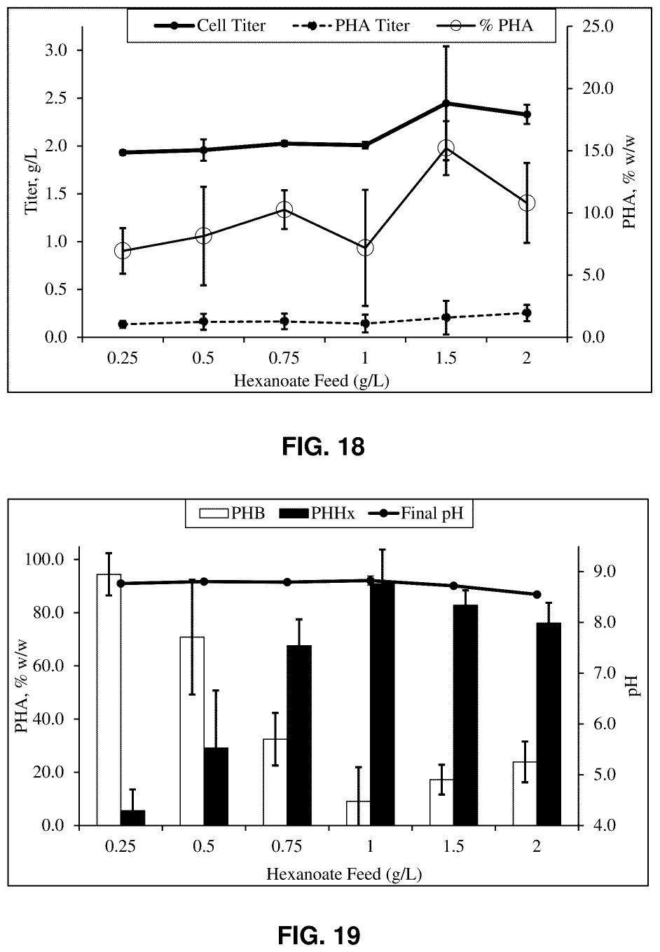

FIG. 18 shows cell titer and PHA titer (lines) and % PHA inclusion (markers) after 48 h of E. coli ptsG STJ4 shake flask growth with varying concentrations of sodium hexanoate starting substrate, plus Brij-35.

FIG. 19 shows PHB-co-PHHx comonomer ratios (bars) and final pH (line) after 48 h of E. coli ptsG STJ4 shake flask growth with varying concentrations of sodium hexanoate starting substrate, plus Brij-35.

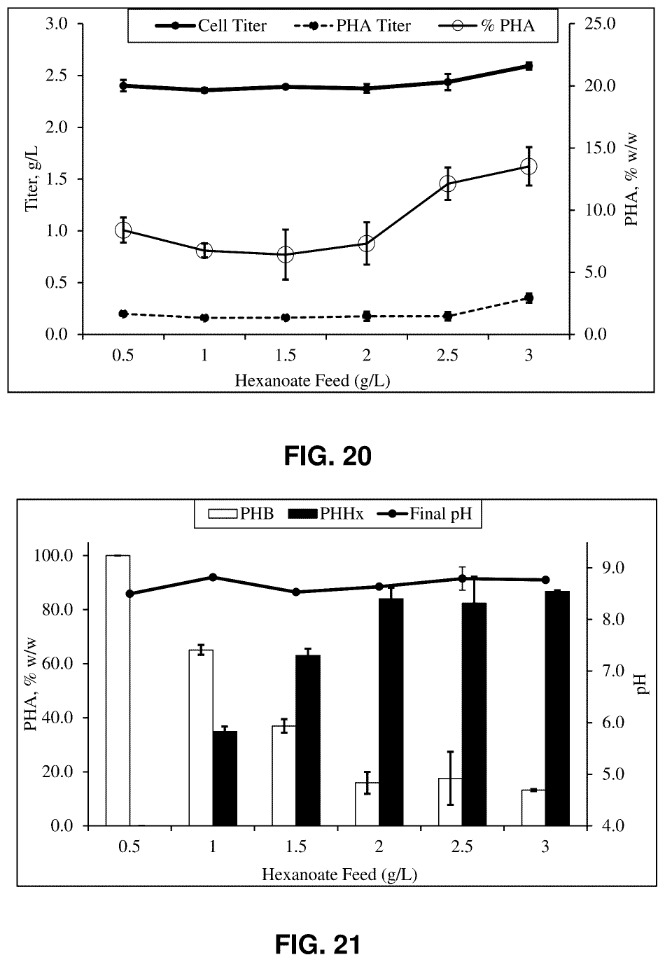

FIG. 20 shows cell titer and PHA titer (lines) and % PHA inclusion (markers) after 48 h of E. coli ptsG STJ4 shake flask growth with varying concentrations of sodium hexanoate starting substrate.

FIG. 21 shows PHB-co-PHHx comonomer ratios (bars) and final pH (line) after 48 h of E. coli ptsG STJ4 shake flask growth with varying concentrations of sodium hexanoate starting substrate.

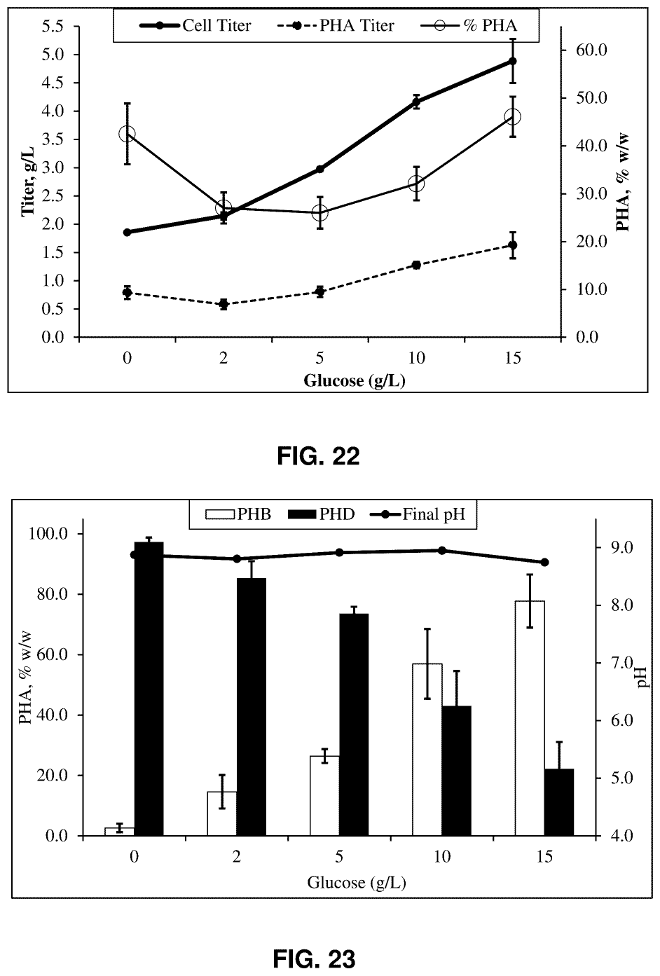

FIG. 22 shows cell titer and PHA titer (lines) and % PHA inclusion (markers) after 48 h of E. coli ptsG STJ4 shake flask growth with varying concentrations of glucose addition at t=7 hours.

FIG. 23 shows PHB-co-PHD comonomer ratios (bars) and final pH (line) after 48 h of E. coli ptsG STJ4 shake flask growth with varying concentrations of glucose addition at t=7 hours.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

Nucleic Acid Sequence Listings

SEQ ID NO: 1 is a primer:
attggtaccc aactgacaac ccggagagt
SEQ ID NO: 2 is a primer:
attgatatcc tggcagttta cgcgagtg
SEQ ID NO: 3 is a nucleotide
sequence for the pBBRSTQKABJ4 plasmid:
ctcgggccgt ctcttgggct tgatcggcct tcttgcgcat
ctcacgcgct cctgcggcgg cctgtagggc aggctcatac
ccctgccgaa ccgcttttgt cagccggtcg gccacggctt
ccggcgtctc aacgcgcttt gagattccca gcttttcggc
caatccctgc ggtgcatagg cgcgtggctc gaccgcttgc
gggctgatgg tgacgtggcc cactggtggc cgctccaggg
cctcgtagaa cgcctgaatg cgcgtgtgac gtgccttgct
gccctcgatg ccccgttgca gccctagatc ggccacagcg
gccgcaaacg tggtctggtc gcgggtcatc tgcgctttgt
tgccgatgaa ctccttggcc gacagcctgc cgtcctgcgt
cagcggcacc acgaacgcgg tcatgtgcgg gctggtttcg
tcacggtgga tgctggccgt cacgatgcga tccgccccgt
acttgtccgc cagccacttg tgcgccttct cgaagaacgc
cgcctgctgt tcttggctgg ccgacttcca ccattccggg
ctggccgtca tgacgtactc gaccgccaac acagcgtcct
tgcgccgctt ctctggcagc aactcgcgca gtcggcccat
cgcttcatcg gtgctgctgg ccgcccagtg ctcgttctct
ggcgtcctgc tggcgtcagc gttgggcgtc tcgcgctcgc
ggtaggcgtg cttgagactg gccgccacgt tgcccatttt
cgccagcttc ttgcatcgca tgatcgcgta tgccgccatg
cctgcccctc ccttttggtg tccaaccggc tcgacggggg
cagcgcaagg cggtgcctcc ggcgggccac tcaatgcttg
agtatactca ctagactttg cttcgcaaag tcgtgaccgc
ctacggcggc tgcggcgccc tacgggcttg ctctccgggc
ttcgccctgc gcggtcgctg cgctcccttg ccagcccgtg
gatatgtgga cgatggccgc gagcggccac cggctggctc
gcttcgctcg gcccgtggac aaccctgctg gacaagctga
tggacaggct gcgcctgccc acgagcttga ccacagggat
tgcccaccgg ctacccagcc ttcgaccaca tacccaccgg
ctccaactgc gcggcctgcg gccttgcccc atcaattttt
ttaattttct ctggggaaaa gcctccggcc tgcggcctgc
gcgcttcgct tgccggttgg acaccaagtg gaaggcgggt
caaggctcgc gcagcgaccg cgcagcggct tggccttgac
gcgcctggaa cgacccaagc ctatgcgagt gggggcagtc
gaagggcgaa gcccgcccgc ctgccccccg agcctcacgg
cggcgagtgc gggggttcca agggggcagc gccaccttgg
gcaaggccga aggccgcgca gtcgatcaac aagccccgga
ggggccactt tttgccggag ggggagccgc gccgaaggcg
tgggggaacc ccgcaggggt gcccttcttt gggcaccaaa
gaactagata tagggcgaaa tgcgaaagac ttaaaaatca
acaacttaaa aaaggggggt acgcaacagc tcattgcggc
accccccgca atagctcatt gcgtaggtta aagaaaatct
gtaattgact gccactttta cgcaacgcat aattgttgtc
gcgctgccga aaagttgcag ctgattgcgc atggtgccgc
aaccgtgcgg cacccctacc gcatggagat aagcatggcc
acgcagtcca gagaaatcgg cattcaagcc aagaacaagc
ccggtcactg ggtgcaaacg gaacgcaaag cgcatgaggc
gtgggccggg cttattgcga ggaaacccac ggcggcaatg
ctgctgcatc acctcgtggc gcagatgggc caccagaacg
ccgtggtggt cagccagaag acactttcca agctcatcgg
acgttctttg cggacggtcc aatacgcagt caaggacttg
gtggccgagc gctggatctc cgtcgtgaag ctcaacggcc
ccggcaccgt gtcggcctac gtggtcaatg accgcgtggc
gtggggccag ccccgcgacc agttgcgcct gtcggtgttc
agtgccgccg tggtggttga tcacgacgac caggacgaat
cgctgttggg gcatggcgac ctgcgccgca tcccgaccct
gtatccgggc gagcagcaac taccgaccgg ccccggcgag
gagccgccca gccagcccgg cattccgggc atggaaccag
acctgccagc cttgaccgaa acggaggaat gggaacggcg
cgggcagcag cgcctgccga tgcccgatga gccgtgtttt
ctggacgatg gcgagccgtt ggagccgccg acacgggtca
cgctgccgcg ccggtagcac ttgggttgcg cagcaacccg
taagtgcgct gttccagact atcggctgta gccgcctcgc
cgccctatac cttgtctgcc tccccgcgtt gcgtcgcggt
gcatggagcc gggccacctc gacctgaatg gaagccggcg
gcacctcgct aacggattca ccgtttttat caggctctgg
gaggcagaat aaatgatcat atcgtcaatt attacctcca
cggggagagc ctgagcaaac tggcctcagg catttgagaa
gcacacggtc acactgcttc cggtagtcaa taaaccggta
aaccagcaat agacataagc ggctatttaa cgaccctgcc
ctgaaccgac gaccgggtcg aatttgcttt cgaatttctg
ccattcatcc gcttattatc acttattcag gcgtagcaac
caggcgttta agggcaccaa taactgcctt aaaaaaatta
cgccccgccc tgccactcat cgcagtacgg cctattggtt
aaaaaatgag ctgatttaac aaaaatttaa cgcgaatttt
aacaaaatat taacgcttac aatttccatt cgccattcag
gctgcgcaac tgttgggaag ggcgatcggt gcgggcctct
tcgctattac gccagctggc gaaaggggga tgtgctgcaa
ggcgattaag ttgggtaacg ccagggtttt cccagtcacg
acgttgtaaa acgacggcca gtgagcgcgc gtaatacgac
tcactatagg gcgaattgga gctccaccgc ggtggcggcc
gctctagaac tagtggatcc cctcgccccc gcgagggccg
cgctgcacga acatggtgct ggctgcgccg ctgccctgat
tctatgccca acaaggcact aagaaaagcg acggggctta
aggaaaaccc ggtgaattgg cgcaaaaagc gaggaatgcc
gcgcgggcag aaacgattcg cgggccttga cggcccgcga
aacgggcggc gaaacgaaac gcccgccgcc ttgtgcgccg
cgctggctgc accgcaatac gcgggcgcca gcgccggctg
ccgactggtt gaaccaggcc ggcaggtcag cccatatgca
ggccgccgtt gagcgagaag tcggcgccgg tcgagaaacc
ggactcctcc gacgacaacc aggcgcagat cgaggcgatc
tcttccggca ggcccaggcg cttgaccggg atcgtcgcga
cgatcttgtc gagcacgtcc tggcggatcg ccttgaccat
gtcggtggcg atatagcccg gagagaccgt gttgacggtc
acgcccttgg tcgccacttc ctgcgccagt gccatggtga
agccatgcag gccggccttg gcggtggagt agttggtctg
gccgaactgg cccttctgcc cgttcaccga cgagatgttg
acgatgcggc cccagccacg gtcggccatg ccgtcgatca
cctgcttggt gacgttgaac agcgaggtca ggttggtgtc
gatcaccgca tcccagtcgg cgcgggtcat cttgcggaac
accacgtcgc gggtgatacc ggcgttgttg atcagcacat
caacctcgcc gacctcggac ttgaccttgt cgaatgcggt
cttggtcgag tcccagtcag ccacattgcc ttccgaggca
atgaaatcga agcccagggc cttctgctgc tccagccact
tttcgcggcg cggcgagttg gggccgcaac cggccaccac
acgaaagcca tccttggcca gccgctggca aatggcggtt
ccgataccac ccatgccgcc ggtcacatac gcaatgcgct
gagtcatgtc cactccttga ttggcttcgt tatcgtcgcc
gggtccgcgc caaccgcgcg cggccccgga aaaccccttc
cttatttgcg ctcgactgcc agcgccacgc ccatgccgcc
gccgatgcac agcgaggcca ggcccttctt cgcgtcacgg
cgcttcatct cgtgcagcag cgtcaccagg atacggcagc
ccgacgcgcc gatcgggtgg ccgatggcga tggcgccgcc
gttcacattg accttggagg tgtcccagcc catctgctgg
tgcaccgcca gcgcctgcgc ggcaaaggcc tcgttgatct
ccatcaggtc caggtcttgc ggggtccact cggcgcgcga
cagggcgcgc ttggaggccg gcaccgggcc catgcccatc
accttgggat cgacaccggc gttggcatag ctcttgatcg
tggccagcgg ggtcaggccc agttccttgg ccttggccgc
cgacatcacc accaccgcgg cggcgccgtc gttcaggccc
gaggcgttgg ccgcggtcac cgtgccggcc ttgtcgaagg
cgggcttgag gccggacatg ctgtccagcg tggcgccctg
gcgcacgaac tcgtcggtct tgaaggccac cgggtcgccc
ttgcgctgcg ggatcagcac cgggacgatc tcttcgtcaa
acttgccggc cttctgcgcg gcttcggcct tgttctgcga
gccgacggcg aactcatcct gcgcctcgcg tgtgatgccg
tattccttgg ccacgttctc ggcggtgatg cccatgtggt
actggttgta cacgtcccac aggccgtcga cgatcatggt
gtcgaccagc ttggcatcgc ccatgcggaa accatcgcgc
gagcccggca gcacgtgcgg ggcggcgctc atgttttcct
ggccgccggc caccacgatc tcggcgtcgc ccgccatgat
cgcgttggcg gccagcatca cggccttcag gcccgagccg
cacaccttgt tgatggtcat ggccggcacc atcgccggca
ggccggcctt gatcgcggcc tggcgtgcgg ggttctggcc
cgaaccggcg gtcagcacct ggcccatgat gacttcgctc
acctgctccg gcttgacgcc ggcgcgctcc agcgcggcct
tgatgaccac ggcacccagt tccggtgccg ggatcttggc
cagcgagccg ccaaacttgc cgaccgcggt gcgggcggcg
gatacgatga caacgtcagt cattgtgtag tcctttcaat
ggaaacggga gggaacctgc agagatccaa cttaacgttc
atgcacatac gtgcccggcg cggcttctcc tgacggatag
gccttgttgc ccaggctggt cggggacttt ttcagtttgc
ccgagcgctc ggcctgccag gcctgccagt gcagccacca
ggagtcggtg tgcttggttg agttttcttg ccactcgttg
gcggtggctg gcatgtcggt gctggtcatg taacgtgatt
tcggattgcc cggcgggttc agaatgctct tgatatgccc
actgctggac agcacgaatt cgaccttgcc accgaacagt
tgcgccgact tgtagcaaga cttccagggc gtgatgtgat
cgttggtgcc ggccagggag tagatgtcgg cagtgacctg
tttgaggtcg atcggcgtgc cgctcacttc gagtgcattg
gcgcgcacca gtgggttatt tttgaacatt tcgatcagat
cgccgtggaa cgcagcaggc aaccgggtgg tgtcgttgtt
ccagaaaaga atgtcgaaga ccggtggctc gttacccagc
aggtagttgt tgacccagta gttccagatc aggtcgttag
ggcgcatcca ggcgaagact ttggccatgt cgcggccttc
cagcacgccg gcctgatacg agtgacgctt ggcagcttcc
agggttttct catcgacgaa cagtgcaacc tgggagtcga
gggtggtgtc gagcacggtg accaaaaggg tcagggcatt
gaccttcttc tcgccgagag cggcgtagtg acccagcagc
gcggtgcagg taatgccacc ggagcaggcg ccgagcatgt
tgatgtcttt gctgccggtg atggcggaaa ctacgtcgac
ggcttctttg agcgcatcga tgtaagtcga cagaccccac
tcacgctggg ccttggtcgg gttgcgccag ctgacgataa
aggtttgctg gttgttgctc aggcagaagc gcgccaggct
tttatccggg ctcaggtcaa acacataaaa cttgttgatc
tgcggtggga ccaccagcag cggtcgctca tgcacctgtt
cggtggtcgg ccggtactgg atcaattcga ggacgtcgtt
gcggaaaacc actgcacctt cagtcgtccc cagactcttg
ccgacttcga aagcgcccat gtccacctgg ctcggcatgc
cgccgttgtt taccaggtcc ttggccagat gtgtgaggcc
gtcgagcagg cttttaccgc cggtttcgaa gaagcgtttg
accgccgccg gattggccgc actgttggtc ggggccatgg
cttcggtcat cagggtgatc acgaagtgag cgcgattgat
gtcctgttcg gacagtttgc tgttgccgat ccagtcgtgg
agttccttgc gccacgccag gtaggtttgt agataacgtt
tgtagagtgg gttctgactc caggcggggt cgttgaaacg
acggtcatcg ctttccggtt gcagcttcga tttgccaaac
atcacgttct tcagctcgat gccaaaatgc gcgacgtgct
tgacgctgtg aatgggttgt ttgatggctt gggttaaaac
cattcgggca gaagtcagca gatcttttcc acgcaggccg
atgacagggt taagccccaa ggtgttttcc gaggcttgac
gattcaagtc atcgctattc ttgttactca tatgtatatc
tccttcttaa agttaaacaa aattatttct agattcgaac
cggctccggg cattgccctg gccggcactt tgcatgggga
gatgctatcc gaatggaccc ggcttgcgcc tcccccaaag
cgggagggtc tgccggcaca tctgccctgg aactggctgg
aagccctcga ccgcacctgc tgcggccgtg gcttgcgtcc
attccgatag cggctcccct tttatccggc aagcgcgaca
ttctcgcatg gagacgccat gcgctatgct tgccggagaa
acctgggaat cgtcagcgat tccgagacat ttgagtccat
tgttgccttg caacgcacgc gctgtcaatg cgggaatccg
cctcggcact gcacgcttcc cgacctaccg gacggtatgc
agcgctcgca tctgccgagg ccccagagca taggcgagaa
ggatgaattt ttgatgtaca tcgtggccat tggctggctc
tacgtggcgc tgatgatggc gatcaccgag cacaacgtgg
tggcaggcgt tgccaccttc ctgatgtatg gcatggcgcc
ggtggcgctg gtgctctaca tcatgggcac gcccggccgc
cgccgacgca aggctgaagc cgagcgcgcg caggcggcca
ggggcaagga cgagtgaggc agcggcgggc tcagtccgcc
agccagacca ggctggccat gcggccggtc acgccatcgc
gccgatagga gtagaagcga ccggcgtcgg ccacggtgca
ggcgtcgccg ccgtagacct cggtacagcc ggcgcgcgcc
aggcgcgtgc gcgccagcgc atagatgtcg gcaaggtact
tgggatcccc cgggctgcag gaattcgata tcaagcttat
cgataccgtc gacctcgagg gggggcccgg taccgagctc
ggatccacta gtaacggccg ccagtgtgct ggaattcagg
attgatatcc tggcagttta cgcgagtgcg ggggatttgt
ggatcggggg ctgcacagca gctccccggg catgtatcag
acaaaacaga gcgacagcga ctcggctata taagcaggct
tctcttcacc ctcaatctcc agcgtggcaa tcgccttgag
tagccactgc cccggctttt tctccaccac ctcgcccagc
tttaccttca gccgaacccg gctgtcgacc ttgaccggct
gaatgaagcg cacgctgtcc agcccgtagt tcaccaccat
cttcagtcct tgcggcagga cgaggatgtc ctcgatcagc
ttggggatca gcgacaaggt caggaaacca tgggcgatcg
tgccgccaaa cggggttttt gccgccttct cagggtcgac
atggatgaac tggaaatcgc cggtcgcctc ggcgaacagg
ttgatgcgct gctggtcgat cttcaaccac tccgagtggc
ccagctcctt gccaacgtac tgcgaaagct ctgtaaccgg
tacatggggc atcgcggact ctccgggttg tcagttgggt
acccagcttt tgttcccttt agtgagggtt aattgcgcgc
ttggcgtaat catggtcata gctgtttcct gtgtgaaatt
gttatccgct cacaattcca cacaacatac gagccggaag
cataaagtgt aaagcctggg gtgcctaatg agtgagctaa
ctcacattaa ttgcgttgcg ctcactgccc gctttccagt
cgggaaacct gtcgtgccag ctgcattaat gaatcggcca
acgcgcgggg agaggcggtt tgcgtattgg gcgcatgcat
aaaaactgtt gtaattcatt aagcattctg ccgacatgga
agccatcaca aacggcatga tgaacctgaa tcgccagcgg
catcagcacc ttgtcgcctt gcgtataata tttgcccatg
ggggtgggcg aagaactcca gcatgagatc cccgcgctgg
aggatcatcc agccggcgtc ccggaaaacg attccgaagc
ccaacctttc atagaaggcg gcggtggaat cgaaatctcg
tgatggcagg ttgggcgtcg cttggtcggt catttcgaac
cccagagtcc cgctcagaag aactcgtcaa gaaggcgata
gaaggcgatg cgctgcgaat cgggagcggc gataccgtaa
agcacgagga agcggtcagc ccattcgccg ccaagctctt
cagcaatatc acgggtagcc aacgctatgt cctgatagcg
gtccgccaca cccagccggc cacagtcgat gaatccagaa
aagcggccat tttccaccat gatattcggc aagcaggcat
cgccatgggt cacgacgaga tcctcgccgt cgggcatgcg
cgccttgagc ctggcgaaca gttcggctgg cgcgagcccc
tgatgctctt cgtccagatc atcctgatcg acaagaccgg
cttccatccg agtacgtgct cgctcgatgc gatgtttcgc
ttggtggtcg aatgggcagg tagccggatc aagcgtatgc
agccgccgca ttgcatcagc catgatggat actttctcgg
caggagcaag gtgagatgac aggagatcct gccccggcac
ttcgcccaat agcagccagt cccttcccgc ttcagtgaca
acgtcgagca cagctgcgca aggaacgccc gtcgtggcca
gccacgatag ccgcgctgcc tcgtcctgca gttcattcag
ggcaccggac aggtcggtct tgacaaaaag aaccgggcgc
ccctgcgctg acagccggaa cacggcggca tcagagcagc
cgattgtctg ttgtgcccag tcatagccga atagcctctc
cacccaagcg gccggagaac ctgcgtgcaa tccatcttgt
tcaatcatgc gaaacgatcc tcatcctgtc tcttgatcag
atcttgatcc cctgcgccat cagatccttg gcggcaagaa
agccatccag tttactttgc agggcttccc aaccttacca
gagggcgccc cagctggcaa ttccggttcg cttgctgtcc
ataaaaccgc ccagtctagc tatcgccatg taagcccact
gcaagctacc tgctttctct ttgcgcttgc gttttccctt
gtccagatag cccagtagct gacattcatc ccaggtggca
cttttcgggg aaatgtgcgc gcccgcgttc ctgctggcgc
tgggcctgtt tctggcgctg gacttcccgc tgttccgtca
gcagcttttc gcccacggcc ttgatgatcg cggcggcctt
ggcctgcata tcccgattca acggccccag ggcgtccaga
acgggcttca ggcgctcccg aaggt
SEQ ID NO: 4 pBBR1 oriV:
Replication origin for the pBBR1 vector,
includes pBBR1 Rep protein:
gcggccaccg gctggctcgc ttcgctcggc ccgtggacaa
ccctgctgga caagctgatg gacaggctgc gcctgcccac
gagcttgacc acagggattg cccaccggct acccagcctt
cgaccacata cccaccggct ccaactgcgc ggcctgcggc
cttgccccat caattttttt aattttctct ggggaaaagc
ctccggcctg cggcctgcgc gcttcgcttg ccggttggac
accaagtgga aggcgggtca aggctcgcgc agcgaccgcg
cagcggcttg gccttgacgc gcctggaacg acccaagcct
atgcgagtgg gggcagtcga agggcgaagc ccgcccgcct
gccccccgag cctcacggcg gcgagtgcgg gggttccaag
ggggcagcgc caccttgggc aaggccgaag gccgcgcagt
cgatcaacaa gccccggagg ggccactttt tgccggaggg
ggagccgcgc cgaaggcgtg ggggaacccc gcaggggtgc
ccttctttgg gcaccaaaga actagatata gggcgaaatg
cgaaagactt aaaaatcaac aacttaaaaa aggggggtac
gcaacagctc attgcggcac cccccgcaat agctcattgc
gtaggttaaa gaaaatctgt aattgactgc cacttttacg
caacgcataa ttgttgtcgc gctgccgaaa agttgcagct
gattgcgcat ggtgccgcaa ccgtgcggca cccctaccgc
atggagataa gc
SEQ ID NO: 5 pBBR1 Rep:
Codes for the replication protein for
the pBBR1 vector:
atggccacgc agtccagaga aatcggcatt caagccaaga
acaagcccgg tcactgggtg caaacggaac gcaaagcgca
tgaggcgtgg gccgggctta ttgcgaggaa acccacggcg
gcaatgctgc tgcatcacct cgtggcgcag atgggccacc
agaacgccgt ggtggtcagc cagaagacac tttccaagct
catcggacgt tctttgcgga cggtccaata cgcagtcaag
gacttggtgg ccgagcgctg gatctccgtc gtgaagctca
acggccccgg caccgtgtcg gcctacgtgg tcaatgaccg
cgtggcgtgg ggccagcccc gcgaccagtt gcgcctgtcg
gtgttcagtg ccgccgtggt ggttgatcac gacgaccagg
acgaatcgct gttggggcat ggcgacctgc gccgcatccc
gaccctgtat ccgggcgagc agcaactacc gaccggcccc
ggcgaggagc cgcccagcca gcccggcatt ccgggcatgg
aaccagacct gccagccttg accgaaacgg aggaatggga
acggcgcggg cagcagcgcc tgccgatgcc cgatgagccg
tgttttctgg acgatggcga gccgttggag ccgccgacac
gggtcacgct gccgcgccgg tag
SEQ ID NO: 6 phaB: Codes for C. necator
PhaB acetoacetyl-CoA reductase:
tcagcccata tgcaggccgc cgttgagega gaagteggeg
ceggtcgaga aaccggactc ctccgacgac aaccaggcgc
agatcgaggc gatctcttcc ggcaggccca ggcgcttgac
cgggatcgtc gcgacgatct tgtcgagcac gtcctggcgg
atcgccttga ccatgtcggt ggcgatatag cccggagaga
ccgtgttgac ggtcacgccc ttggtcgcca cttcctgcgc
cagtgccatg gtgaagccat gcaggccggc cttggcggtg
gagtagttgg tctggccgaa ctggcccttc tgcccgttca
ccgacgagat gttgacgatg cggccccagc cacggteggc
catgccgtcg atcacctgct tggtgacgtt gaacagcgag
gtcaggttgg tgtcgatcac cgcatcccag tcggcgcggg
tcatcttgeg gaacaccacg tegegggtga taccggegtt
gttgatcage acatcaacct cgccgacctc ggacttgacc
ttgtcgaatg cggtcttggt cgagtcccag tcagccacat
tgccttcega ggcaatgaaa tegaagccca gggccttctg
ctgctccage cacttttcgc ggcgcggcga gttggggccg
caaccggcca ccacacgaaa gccatccttg gccagccgct
ggcaaatggc ggttccgata ccacccatgc cgccggtcac
atacgcaatg cgctgagtca t
SEQ ID NO: 7 phaA: Codes for C. necator
PhaA β-ketothiolase:
ttatttgcgc tcgactgcca gcgccacgcc catgccgccg
ccgatgcaca gcgaggccag gcccttcttc gcgtcacggc
gcttcatctc gtgcagcagc gtcaccagga tacggcagcc
cgacgcgccg atcgggtggc cgatggcgat ggcgccgccg
ttcacattga ccttggaggt gtcccagccc atctgctggt
gcaccgccag cgcctgcgcg gcaaaggcct cgttgatctc
catcaggtcc aggtcttgcg gggtccactc ggcgcgcgac
agggcgcgct tggaggccgg caccgggccc atgcccatca
ccttgggatc gacaccggcg ttggcatagc tcttgatcgt
ggccagcggg gtcaggccca gttccttggc cttggccgcc
gacatcacca ccaccgcggc ggcgccgtcg ttcaggcccg
aggcgttggc cgcggtcacc gtgccggcct tgtcgaaggc
gggcttgagg ccggacatgc tgtccagcgt ggcgccctgg
cgcacgaact cgtcggtctt gaaggccacc gggtcgccct
tgcgctgcgg gatcagcacc gggacgatct cttcgtcaaa
cttgccggcc ttctgcgcgg cttcggcctt gttctgcgag
ccgacggcga actcatcctg cgcctcgcgt gtgatgccgt
attccttggc cacgttctcg gcggtgatgc ccatgtggta
ctggttgtac acgtcccaca ggccgtcgac gatcatggtg
tcgaccagct tggcatcgcc catgcggaaa ccatcgcgcg
agcccggcag cacgtgcggg gcggcgctca tgttttcctg
gccgccggcc accacgatct cggcgtcgcc cgccatgatc
gcgttggcgg ccagcatcac ggccttcagg cccgagccgc
acaccttgtt gatggtcatg gccggcacca tcgccggcag
gccggccttg atcgcggcct ggcgtgcggg gttctggccc
gaaccggcgg tcagcacctg gcccatgatg acttcgctca
cctgctccgg cttgacgccg gcgcgctcca gcgcggcctt
gatgaccacg gcacccagtt ccggtgccgg gatcttggcc
agcgagccgc caaacttgcc gaccgcggtg cgggcggcgg
atacgatgac aacgtcagtc at
SEQ ID NO: 8 phaC1(STQK): Codes for a
Pseudomonas sp. 61-3 PhaC PHA synthase, which
has been modified for enhanced uptake of SCL
acyl-CoA substrates:
acgttcatgc acatacgtgc ccggcgcggc ttctcctgac
ggataggcct tgttgcccag gctggtcggg gactttttca
gtttgcccga gcgctcggcc tgccaggcct gccagtgcag
ccaccaggag tcggtgtgct tggttgagtt ttcttgccac
tcgttggcgg tggctggcat gtcggtgctg gtcatgtaac
gtgatttcgg attgcccggc gggttcagaa tgctcttgat
atgcccactg ctggacagca cgaattcgac cttgccaccg
aacagttgcg ccgacttgta gcaagacttc cagggcgtga
tgtgatcgtt ggtgccggcc agggagtaga tgtcggcagt
gacctgtttg aggtcgatcg gcgtgccgct cacttcgagt
gcattggcgc gcaccagtgg gttatttttg aacatttcga
tcagatcgcc gtggaacgca gcaggcaacc gggtggtgtc
gttgttccag aaaagaatgt cgaagaccgg tggctcgtta
cccagcaggt agttgttgac ccagtagttc cagatcaggt
cgttagggcg catccaggcg aagactttgg ccatgtcgcg
gccttccagc acgccggcct gatacgagtg acgcttggca
gcttccaggg ttttctcatc gacgaacagt gcaacctggg
agtcgagggt ggtgtcgagc acggtgacca aaagggtcag
ggcattgacc ttcttctcgc cgagagcggc gtagtgaccc
agcagcgcgg tgcaggtaat gccaccggag caggcgccga
gcatgttgat gtctttgctg ccggtgatgg cggaaactac
gtcgacggct tctttgagcg catcgatgta agtcgacaga
ccccactcac gctgggcctt ggtcgggttg cgccagctga
cgataaaggt ttgctggttg ttgctcaggc agaagcgcgc
caggctttta tccgggctca ggtcaaacac ataaaacttg
ttgatctgcg gtgggaccac cagcagcggt cgctcatgca
cctgttcggt ggtcggccgg tactggatca attcgaggac
gtcgttgcgg aaaaccactg caccttcagt cgtccccaga
ctcttgccga cttcgaaagc gcccatgtcc acctggctcg
gcatgccgcc gttgtttacc aggtccttgg ccagatgtgt
gaggccgtcg agcaggcttt taccgccggt ttcgaagaag
cgtttgaccg ccgccggatt ggccgcactg ttggtcgggg
ccatggcttc ggtcatcagg gtgatcacga agtgagcgcg
attgatgtcc tgttcggaca gtttgctgtt gccgatccag
tcgtggagtt ccttgcgcca cgccaggtag gtttgtagat
aacgtttgta gagtgggttc tgactccagg cggggtcgtt
gaaacgacgg tcatcgcttt ccggttgcag cttcgatttg
ccaaacatca cgttcttcag ctcgatgcca aaatgcgcga
cgtgcttgac gctgtgaatg ggttgtttga tggcttgggt
taaaaccatt cgggcagaag tcagcagatc ttttccacgc
aggccgatga cagggttaag ccccaaggtg ttttccgagg
cttgacgatt caagtcatcg ctattcttgt tactcat
SEQ ID NO: 9 R. eutropha promoter:
A constitutive promoter from C. necator
that recruits
RNA polymerase to begin transcription:
gagacatttg agtccattgt tgccttgcaa cgcacgcgct
gtcaatgcgg
SEQ ID NO: 10 Codes for Pseudomonas putida
PhaJ4 (R)-specific enoyl-CoA hydratase:
tcagacaaaa cagagcgaca gcgactcggc tatataagca
ggcttctctt caccctcaat ctccagcgtg gcaatcgcct
tgagtagcca ctgccccggc tttttctcca ccacctcgcc
cagctttacc ttcagccgaa cccggctgtc gaccttgacc
ggctgaatga agcgcacgct gtccagcccg tagttcacca
ccatcttcag tccttgcggc aggacgagga tgtcctcgat
cagcttgggg atcagcgaca aggtcaggaa accatgggcg
atcgtgccgc caaacggggt ttttgccgcc ttctcagggt
cgacatggat gaactggaaa tcgccggtcg cctcggcgaa
caggttgatg cgctgctggt cgatcttcaa ccactccgag
tggcccagct ccttgccaac gtactgcgaa agctctgtaa
ccggtacatg gggcat
SEQ ID NO: 11 is a forward primer
sequence (5' to 3') pKD3.F.ptsG:
acgtaaaaaa agcacccata
ctcaggagca ctctcaattg tgtaggctgg agctgcttc
SEQ ID NO: 12 is a reverse primer
sequence (5' to 3') pK13.R.ptsG:
agccatctgg ctgccttagt
ctccccaacg tcttacggaa tgggaattag ccatggtcc
SEQ ID NO: 13 is a forward primer
sequence (5' to 3') ptsG.F.check:
cctgtacacg gegaggctct.
SEQ ID NO: 14 is a reverse primer
sequence (5' to 3') ptsG.R.check:
aataacacct gtaaaaaagg
c4agcc.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

What is claimed is:

1-96. (canceled).

97. A nucleic acid construct comprising:
at least one gene selected from the group consisting of a phaJ4 gene, a phaA gene, a phaB gene, a phaC1 gene; and
at least one nucleic acid sequence that encodes at least one protein selected from the group consisting of an enoyl-CoA hydratase 2, a β-ketothiolase, an acetoacetyl-CoA reductase, a type II polyhydroxyalkanoate synthase, or combinations thereof.

98. The nucleic acid construct of claim 97, further comprising a promoter sequence operably linked to the nucleic acid construct, wherein the at least one nucleic acid sequence comprises a cDNA.

99. The nucleic acid construct of claim 98, wherein the promoter sequence is selected from the group consisting of a Ralstonia eutropha promoter and a Cupriavidus necator constitutive promoter.

100. The nucleic acid construct of claim 97, further comprising a phosphotransferase gene repressor.

101. The nucleic acid construct of claim 97, wherein the at least one nucleic acid sequence comprises one or more coding sequences involved in a biosynthesis of polyhydroxyalkanoate (PHA) homopolymers or PHA copolymers.

102. The nucleic acid construct of claim 97, wherein the nucleic acid construct is contained on an extrachromosomal element or plasmid.

103. The nucleic acid construct of claim 97, contained within a live host cell.

104. A method of forming at least one of a polyhydroxyalkanoate (PHA) homopolymer and a PHA copolymer, comprising:
cultivating a recombinant E. coli cell in a medium conducive for a production of the at least one of the PHA homopolymer and the PHA copolymer,
wherein the recombinant E. coli cell comprises:
a disrupted endogenous β-oxidation pathway having at least one of a genetically disrupted endogenous fadB gene and a genetically disrupted fadJ gene, and
a nucleic acid construct comprising:
(a) at least one phaJ4 gene, cDNA, or nucleic acid encoding active enoyl-CoA hydratase 2,
(b) at least one phaA gene, cDNA, or nucleic acid encoding active β-ketothiolase,
(c) at least one phaB gene, cDNA, or nucleic acid encoding active acetoacetyl-CoA reductase, and
(d) at least one phaC1 gene, cDNA, or nucleic acid encoding active type II polyhydroxyalkanoate synthase,
wherein said cultivating occurs under conditions suitable for bioconverting a substrate to that at least one of the PHA homopolymer and the PHA copolymer.

105. The method of claim 104, wherein the substrate comprises a fatty acid substrate comprising 6-14 carbons.

106. The method of claim 105, wherein the substrate further comprises at least one of glucose, xylose, arabinose, mannose, and galactose.

107. The method of claim 104, wherein the substrate is derived from lignocellulosic waste.

108. The method of claim 104, wherein the recombinant E. coli cell contains 5-10 copies of the nucleic acid construct.

109. The method of claim 104, wherein the nucleic acid construct is contained on an extrachromosomal element.

110. The method of claim 104, wherein the E. coli cell has impaired phosphotranferse function resulting from a deletion of ptsG.

111. A genetically engineered E. coli cell comprising:
a disrupted endogenous β-oxidation pathway comprising at least one of a genetically disrupted endogenous fadB gene and a genetically disrupted fadJ gene, and
at least one nucleic acid comprising:
at least one gene encoding active enoyl-CoA hydratase 2;
at least one gene encoding active β-ketothiolase;
at least one gene encoding active acetoacetyl-CoA reductase; and
at least one gene encoding active type II polyhydroxyalkanoate synthase,
wherein the at least one nucleic acid causes the genetically engineered E. coli cell to have increased activity of enoyl-CoA hydratase 2, β-ketothiolase, acetoacetyl-CoA reductase, and type II polyhydroxyalkanoate synthase, with respect to a wild type E. coli lacking the at least one nucleic acid construct.

112. The genetically engineered E. coli cell of claim 111, wherein the nucleic acid construct comprises:
at least one phaJ4 gene encoding active enoyl-CoA hydratase 2,
at least one phaA gene encoding active β-ketothiolase,
at least one phaB gene encoding active acetoacetyl-CoA reductase, and
at least one phaC1 (STQK) gene encoding active type II polyhydroxyalkanoate synthase.

113. The genetically engineered E. coli cell of claim 112, wherein the nucleic acid construct comprises a Cupriavidus necator constitutive promoter operatively linked to each of:
the at least one phaA gene encoding active β-ketothiolase,
the at least one phaB gene encoding active acetoacetyl-CoA reductase, and
the at least one phaC1 gene encoding active type II polyhydroxyalkanoate synthase.

114. The genetically engineered E. coli cell of claim 112, wherein the nucleic acid construct comprises a cDNA comprising coding sequences for biosynthesis enzymes for synthesis of homopolymers or copolymers of polyhydroxyalkanoate (PHA).

115. The genetically engineered E. coli cell of claim 112, having reduced phosphotranferase function with respect to a wild type E. coli cell,
wherein:
the active enoyl-CoA hydratase 2 converts one or more enoyl-CoA intermediates to one or more (R)-3-hydroxyacyl-CoA equivalents;
the active β-ketothiolase and active acetoacetyl-CoA reductase condense 2 acetyl-CoAs to (R)-3-hydroxybutyryl-CoA; and
the active type II polyhydroxyalkanoate synthase converts one or more (R)-3-hydroxyacyl-CoA equivalents and (R)-3-hydroxybutyryl-CoA into PHA or PHB-co-MCL PHA copolymers.

116. The genetically engineered E. coli cell of claim 111, wherein:
the disrupted endogenous β-oxidation pathway results in a buildup of one or more enoyl-CoA intermediates;
the at least one gene encoding active enoyl-CoA hydratase 2 comprises a genetically upregulated phaJ4 gene encoding active enoyl-CoA hydratase 2 with the upregulation resulting in increased (R)-3-hydroxyacyl-CoA equivalents, wherein the upregulation is produced by introducing at least one phaJ4 gene into the E. coli strain;
the at least one gene encoding active β-ketothiolase comprises a genetically upregulated phaA gene encoding active β-ketothiolase the upregulation resulting in increased (R)-3-hydroxybutyryl-CoA, wherein the upregulation is produced by introducing at least one phaA gene into the E. coli strain;
the at least one gene encoding active acetoacetyl-CoA reductase comprises a genetically upregulated phaB gene encoding active acetoacetyl-CoA reductase the upregulation resulting in increased (R)-3-hydroxybutyryl-CoA, wherein the upregulation is produced by introducing at least one phaB gene into the E. coli strain; and
the at least one gene encoding active type II polyhydroxyalkanoate synthase comprises a genetically upregulated phaC1 gene encoding active type II polyhydroxyalkanoate synthase, the upregulation resulting in increased polyhydroxyalkanoate (PHA), wherein the upregulation is produced by introducing at least one phaC1 gene into the E. coli strain,
wherein said E. coli strain is capable of bioconverting a substrate into at least one of PHA homopolymers and PHA copolymers.


SEQUENCE LISTING (14 sequences)

SEQ ID NO: 1 - DNA, 29 bp, synthetic construct
  misc_feature 1..29: note = J4.F.KpnI forward primer
  source 1..29: mol_type = other DNA
attggtacccaactgacaacccggagagt

SEQ ID NO: 2 - DNA, 28 bp, synthetic construct
  misc_feature 1..28: note = J4.R.EcoRV reverse primer
  source 1..28: mol_type = other DNA
attgatatcctggcagtttacgcgagtg

SEQ ID NO: 3 - DNA, 10625 bp, synthetic construct
  misc_feature 1..10625: note = pBBRSTQKABJ4 plasmid
  source 1..10625: mol_type = other DNA
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaagggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcacccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtacggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccctcgcccccgcgagggccgcgctgcacgaacatggtgctggctgcgccgctgccctgattctatgcccaacaaggcactaagaaaagcgacggggcttaaggaaaacccggtgaattggcgcaaaaagcgaggaatgccgcgcgggcagaaacgattcgcgggccttgacggcccgcgaaacgggcggcgaaacgaaacgcccgccgccttgtgcgccgcgctggctgcaccgcaatacgcgggcgccagcgccggctgccgactggttgaaccaggccggcaggtcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcatgtccactccttgattggcttcgttatcgtcgccgggtccgcgccaaccgcgcgcggccccggaaaaccccttccttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcattgtgtagtcctttcaatggaaacgggagggaacctgcagagatccaacttaacgttcatgcacatacgtgcccggcgcggcttctcctgacggataggccttgttgcccaggctggtcggggactttttcagtttgcccgagcgctcggcctgccaggcctgccagtgcagccaccaggagtcggtgtgcttggttgagttttcttgccactcgttggcggtggctggcatgtcggtgctggtcatgtaacgtgatttcggattgcccggcgggttcagaatgctcttgatatgcccactgctggacagcacgaattcgaccttgccaccgaacagttgcgccgacttgtagcaagacttccagggcgtgatgtgatcgttggtgccggccagggagtagatgtcggcagtgacctgtttgaggtcgatcggcgtgccgctcacttcgagtgcattggcgcgcaccagtgggttatttttgaacatttcgatcagatcgccgtggaacgcagcaggcaaccgggtggtgtcgttgttccagaaaagaatgtcgaagaccggtggctcgttacccagcaggtagttgttgacccagtagttccagatcaggtcgttagggcgcatccaggcgaagactttggccatgtcgcggccttccagcacgccggcctgatacgagtgacgcttggcagcttccagggttttctcatcgacgaacagtgcaacctgggagtcgagggtggtgtcgagcacggtgaccaaaagggtcagggcattgaccttcttctcgccgagagcggcgtagtgacccagcagcgcggtgcaggtaatgccaccggagcaggcgccgagcatgttgatgtctttgctgccggtgatggcggaaactacgtcgacggcttctttgagcgcatcgatgtaagtcgacagaccccactcacgctgggccttggtcgggttgcgccagctgacgataaaggtttgctggttgttgctcaggcagaagcgcgccaggcttttatccgggctcaggtcaaacacataaaacttgttgatctgcggtgggaccaccagcagcggtcgctcatgcacctgttcggtggtcggccggtactggatcaattcgaggacgtcgttgcggaaaaccactgcaccttcagtcgtccccagactcttgccgacttcgaaagcgcccatgtccacctggctcggcatgccgccgttgtttaccaggtccttggccagatgtgtgaggccgtcgagcaggcttttaccgccggtttcgaagaagcgtttgaccgccgccggattggccgcactgttggtcggggccatggcttcggtcatcagggtgatcacgaagtgagcgcgattgatgtcctgttcggacagtttgctgttgccgatccagtcgtggagttccttgcgccacgccaggtaggtttgtagataacgtttgtagagtgggttctgactccaggcggggtcgttgaaacgacggtcatcgctttccggttgcagcttcgatttgccaaacatcacgttcttcagctcgatgccaaaatgcgcgacgtgcttgacgctgtgaatgggttgtttgatggcttgggttaaaaccattcgggcagaagtcagcagatcttttccacgcaggccgatgacagggttaagccccaaggtgttttccgaggcttgacgattcaagtcatcgctattcttgttactcatatgtatatctccttcttaaagttaaacaaaattatttctagattcgaaccggctccgggcattgccctggccggcactttgcatggggagatgctatccgaatggacccggcttgcgcctcccccaaagcgggagggtctgccggcacatctgccctggaactggctggaagccctcgaccgcacctgctgcggccgtggcttgcgtccattccgatagcggctccccttttatccggcaagcgcgacattctcgcatggagacgccatgcgctatgcttgccggagaaacctgggaatcgtcagcgattccgagacatttgagtccattgttgccttgcaacgcacgcgctgtcaatgcgggaatccgcctcggcactgcacgcttcccgacctaccggacggtatgcagcgctcgcatctgccgaggccccagagcataggcgagaaggatgaatttttgatgtacatcgtggccattggctggctctacgtggcgctgatgatggcgatcaccgagcacaacgtggtggcaggcgttgccaccttcctgatgtatggcatggcgccggtggcgctggtgctctacatcatgggcacgcccggccgccgccgacgcaaggctgaagccgagcgcgcgcaggcggccaggggcaaggacgagtgaggcagcggcgggctcagtccgccagccagaccaggctggccatgcggccggtcacgccatcgcgccgataggagtagaagcgaccggcgtcggccacggtgcaggcgtcgccgccgtagacctcggtacagccggcgcgcgccaggcgcgtgcgcgccagcgcatagatgtcggcaaggtacttgggatcccccgggctgcaggaattcgatatcaagcttatcgataccgtcgacctcgagggggggcccggtaccgagctcggatccactagtaacggccgccagtgtgctggaattcaggattgatatcctggcagtttacgcgagtgcgggggatttgtggatcgggggctgcacagcagctccccgggcatgtatcagacaaaacagagcgacagcgactcggctatataagcaggcttctcttcaccctcaatctccagcgtggcaatcgccttgagtagccactgccccggctttttctccaccacctcgcccagctttaccttcagccgaacccggctgtcgaccttgaccggctgaatgaagcgcacgctgtccagcccgtagttcaccaccatcttcagtccttgcggcaggacgaggatgtcctcgatcagcttggggatcagcgacaaggtcaggaaaccatgggcgatcgtgccgccaaacggggtttttgccgccttctcagggtcgacatggatgaactggaaatcgccggtcgcctcggcgaacaggttgatgcgctgctggtcgatcttcaaccactccgagtggcccagctccttgccaacgtactgcgaaagctctgtaaccggtacatggggcatcgcggactctccgggttgtcagttgggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 4 - DNA, 772 bp, synthetic construct
  misc_feature 1..772: note = 4 pBBR1 oriV: Replication origin for the pBBR1 vector, includespBBR1 Rep protein
  source 1..772: mol_type = other DNA
gcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaagggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcacccctaccgcatggagataagc

SEQ ID NO: 5 - DNA, 663 bp, synthetic construct
  misc_feature 1..663: note = 5 pBBR1 Rep (replication protein for pBBR1 vector)
  source 1..663: mol_type = other DNA
atggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtag

SEQ ID NO: 6 - DNA, 741 bp, Cupriavidus necator
  source 1..741: mol_type = genomic DNA
tcagcccatatgcaggccgccgttgagcgagaagtcggcgccggtcgagaaaccggactcctccgacgacaaccaggcgcagatcgaggcgatctcttccggcaggcccaggcgcttgaccgggatcgtcgcgacgatcttgtcgagcacgtcctggcggatcgccttgaccatgtcggtggcgatatagcccggagagaccgtgttgacggtcacgcccttggtcgccacttcctgcgccagtgccatggtgaagccatgcaggccggccttggcggtggagtagttggtctggccgaactggcccttctgcccgttcaccgacgagatgttgacgatgcggccccagccacggtcggccatgccgtcgatcacctgcttggtgacgttgaacagcgaggtcaggttggtgtcgatcaccgcatcccagtcggcgcgggtcatcttgcggaacaccacgtcgcgggtgataccggcgttgttgatcagcacatcaacctcgccgacctcggacttgaccttgtcgaatgcggtcttggtcgagtcccagtcagccacattgccttccgaggcaatgaaatcgaagcccagggccttctgctgctccagccacttttcgcggcgcggcgagttggggccgcaaccggccaccacacgaaagccatccttggccagccgctggcaaatggcggttccgataccacccatgccgccggtcacatacgcaatgcgctgagtcat

SEQ ID NO: 7 - DNA, 1182 bp, Cupriavidus necator
  source 1..1182: mol_type = genomic DNA
ttatttgcgctcgactgccagcgccacgcccatgccgccgccgatgcacagcgaggccaggcccttcttcgcgtcacggcgcttcatctcgtgcagcagcgtcaccaggatacggcagcccgacgcgccgatcgggtggccgatggcgatggcgccgccgttcacattgaccttggaggtgtcccagcccatctgctggtgcaccgccagcgcctgcgcggcaaaggcctcgttgatctccatcaggtccaggtcttgcggggtccactcggcgcgcgacagggcgcgcttggaggccggcaccgggcccatgcccatcaccttgggatcgacaccggcgttggcatagctcttgatcgtggccagcggggtcaggcccagttccttggccttggccgccgacatcaccaccaccgcggcggcgccgtcgttcaggcccgaggcgttggccgcggtcaccgtgccggccttgtcgaaggcgggcttgaggccggacatgctgtccagcgtggcgccctggcgcacgaactcgtcggtcttgaaggccaccgggtcgcccttgcgctgcgggatcagcaccgggacgatctcttcgtcaaacttgccggccttctgcgcggcttcggccttgttctgcgagccgacggcgaactcatcctgcgcctcgcgtgtgatgccgtattccttggccacgttctcggcggtgatgcccatgtggtactggttgtacacgtcccacaggccgtcgacgatcatggtgtcgaccagcttggcatcgcccatgcggaaaccatcgcgcgagcccggcagcacgtgcggggcggcgctcatgttttcctggccgccggccaccacgatctcggcgtcgcccgccatgatcgcgttggcggccagcatcacggccttcaggcccgagccgcacaccttgttgatggtcatggccggcaccatcgccggcaggccggccttgatcgcggcctggcgtgcggggttctggcccgaaccggcggtcagcacctggcccatgatgacttcgctcacctgctccggcttgacgccggcgcgctccagcgcggccttgatgaccacggcacccagttccggtgccgggatcttggccagcgagccgccaaacttgccgaccgcggtgcgggcggcggatacgatgacaacgtcagtcat

SEQ ID NO: 8 - DNA, 1677 bp, synthetic construct
  misc_feature 1..1677: note = Pseudomonas sp. 61-3 PhaC PHA synthase, modified for enhanceduptake of SCL acyl-CoA substrates
  source 1..1677: mol_type = other DNA
acgttcatgcacatacgtgcccggcgcggcttctcctgacggataggccttgttgcccaggctggtcggggactttttcagtttgcccgagcgctcggcctgccaggcctgccagtgcagccaccaggagtcggtgtgcttggttgagttttcttgccactcgttggcggtggctggcatgtcggtgctggtcatgtaacgtgatttcggattgcccggcgggttcagaatgctcttgatatgcccactgctggacagcacgaattcgaccttgccaccgaacagttgcgccgacttgtagcaagacttccagggcgtgatgtgatcgttggtgccggccagggagtagatgtcggcagtgacctgtttgaggtcgatcggcgtgccgctcacttcgagtgcattggcgcgcaccagtgggttatttttgaacatttcgatcagatcgccgtggaacgcagcaggcaaccgggtggtgtcgttgttccagaaaagaatgtcgaagaccggtggctcgttacccagcaggtagttgttgacccagtagttccagatcaggtcgttagggcgcatccaggcgaagactttggccatgtcgcggccttccagcacgccggcctgatacgagtgacgcttggcagcttccagggttttctcatcgacgaacagtgcaacctgggagtcgagggtggtgtcgagcacggtgaccaaaagggtcagggcattgaccttcttctcgccgagagcggcgtagtgacccagcagcgcggtgcaggtaatgccaccggagcaggcgccgagcatgttgatgtctttgctgccggtgatggcggaaactacgtcgacggcttctttgagcgcatcgatgtaagtcgacagaccccactcacgctgggccttggtcgggttgcgccagctgacgataaaggtttgctggttgttgctcaggcagaagcgcgccaggcttttatccgggctcaggtcaaacacataaaacttgttgatctgcggtgggaccaccagcagcggtcgctcatgcacctgttcggtggtcggccggtactggatcaattcgaggacgtcgttgcggaaaaccactgcaccttcagtcgtccccagactcttgccgacttcgaaagcgcccatgtccacctggctcggcatgccgccgttgtttaccaggtccttggccagatgtgtgaggccgtcgagcaggcttttaccgccggtttcgaagaagcgtttgaccgccgccggattggccgcactgttggtcggggccatggcttcggtcatcagggtgatcacgaagtgagcgcgattgatgtcctgttcggacagtttgctgttgccgatccagtcgtggagttccttgcgccacgccaggtaggtttgtagataacgtttgtagagtgggttctgactccaggcggggtcgttgaaacgacggtcatcgctttccggttgcagcttcgatttgccaaacatcacgttcttcagctcgatgccaaaatgcgcgacgtgcttgacgctgtgaatgggttgtttgatggcttgggttaaaaccattcgggcagaagtcagcagatcttttccacgcaggccgatgacagggttaagccccaaggtgttttccgaggcttgacgattcaagtcatcgctattcttgttactcat

SEQ ID NO: 9 - DNA, 50 bp, Cupriavidus necator
  source 1..50: mol_type = genomic DNA
gagacatttgagtccattgttgccttgcaacgcacgcgctgtcaatgcgg

SEQ ID NO: 10 - DNA, 456 bp, Pseudomonas putida
  source 1..456: mol_type = genomic DNA
tcagacaaaacagagcgacagcgactcggctatataagcaggcttctcttcaccctcaatctccagcgtggcaatcgccttgagtagccactgccccggctttttctccaccacctcgcccagctttaccttcagccgaacccggctgtcgaccttgaccggctgaatgaagcgcacgctgtccagcccgtagttcaccaccatcttcagtccttgcggcaggacgaggatgtcctcgatcagcttggggatcagcgacaaggtcaggaaaccatgggcgatcgtgccgccaaacggggtttttgccgccttctcagggtcgacatggatgaactggaaatcgccggtcgcctcggcgaacaggttgatgcgctgctggtcgatcttcaaccactccgagtggcccagctccttgccaacgtactgcgaaagctctgtaaccggtacatggggcat

SEQ ID NO: 11 - DNA, 59 bp, synthetic construct
  misc_feature 1..59: note = pKD3.F.ptsG forward primer
  source 1..59: mol_type = other DNA
acgtaaaaaaagcacccatactcaggagcactctcaattgtgtaggctggagctgcttc

SEQ ID NO: 12 - DNA, 59 bp, synthetic construct
  misc_feature 1..59: note = pK13.R.ptsG reverse primer
  source 1..59: mol_type = other DNA
agccatctggctgccttagtctccccaacgtcttacggaatgggaattagccatggtcc

SEQ ID NO: 13 - DNA, 20 bp, synthetic construct
  misc_feature 1..20: note = ptsG.F.check forward primer
  source 1..20: mol_type = other DNA
cctgtacacggcgaggctct

SEQ ID NO: 14 - DNA, 25 bp, synthetic construct
  misc_feature 1..25: note = ptsG.R.check reverse primer
  source 1..25: mol_type = other DNA
aataacacctgtaaaaaaggcagcc